SEQUENCE LISTING (2 sequences)

SEQ ID NO: 1 - protein, 25 aa, Influenza A virus
SSDPLVVAASIIGILHLILWILDRL

SEQ ID NO: 2 - protein, 22 aa, Influenza A virus
PLVVAASIIAILHLILWILDRL

(19) United States
(12) Patent Application Publication
(10) Pub. No.: US 2011/0236881 A1
(43) Pub. Date: Sep. 29, 2011

(54) MODULATION OF INFLUENZA VIRUS

(76) Inventors: William F. Degrado, Media, PA (US); Amanda L. Stouffer, Spring City, PA (US); Rudresh Acharya, Philadelphia, PA (US); David Salom-Arbona, Avon, OH (US); Alexei Polishchuk, Philadelphia, PA (US)

(21) Appl. No.: 13/058,930

(22) PCT Filed: Aug. 12, 2009
(86) PCT No.: PCT/US09/53612

§ 371 Date: Jun. 13, 2011

Related U.S. Application Data
(60) Provisional application No. 61/088,030, filed on Aug. 12, 2008.

(51) Int. Cl. C12Q 1/70; G06F 19/00

(52) U.S. Cl. 435/5; 702/19

(57) ABSTRACT

The present invention provides, among other things, methods for the identification of compounds that are capable of modulating the activity of the influenza A virus. For example, the present methods provide platforms for identifying small molecule inhibitors that target the proton transport pathway defined at least in part by two or more of the highly conserved channel residues 27, 30, 31, 34, 37, 41, 44, and 45 of the influenza A M2 protein. In one aspect, the present invention is directed to methods comprising comparing spatial models of a plurality of test compounds with the spatial model of the pathway defined by at least two residues from among residues 27, 30, 31, 34, 37 or 41, 44, and 45 on one or more subunits of the M2 transmembrane protein of the influenza A virus to determine the spatial complementarity of each of the test compounds with the pathway; assessing the ability of the test compounds to bind to the pathway; and, based on the assessed ability of the test compounds to bind the pathway, determining the compound that modulates the activity of influenza A.

### CROSS REFERENCE TO RELATED APPLICATION

This application claims the benefit of priority to U.S. Provisional App. No. 61/088,030, filed Aug. 12, 2008, the entire contents of which are incorporated herein in their entirety.

### STATEMENT OF GOVERNMENT RIGHTS

Research leading to the disclosed invention was funded in part by the U.S. National Institutes of Health, grant numbers NIH P01 HL40387 and P50 HL54500. Accordingly, the United States Government may have certain rights in the invention described herein.

### TECHNICAL FIELD

The present invention pertains to, among other things, modulation of the activity of influenza virus.

### BACKGROUND

The M2 protein is found in the viral envelope of influenza A virus and functions as a highly selective, pH-regulated proton channel important for the life cycle of the virus. Unlike neuraminidase inhibitors, rimantadine and amantadine are anti-viral agents capable of blocking the tetrameric M2 channel. In 2006, the CDC issued an alert instructing clinicians to avoid using M2 ion-channel inhibitors during influenza season due to the extraordinarily high frequency of amantadine resistance in influenza A isolates associated with a single point mutation in the M2 protein, S31N (Hayden F. G., Antiviral Resistance in Influenza Viruses—Implications for Management and Pandemic Response, N Enj J Med, 2006, 354;8). The drug-binding site is lined by residues that are mutated in amantadine-resistant viruses. Grambas, S., Bennett, M S. & Hay, A. J. Influence of amantadine resistance mutations on the pH regulatory function of the M2 protein of influenza A viruses. Virology 191, 541-549 (1992); Bright, R. A., Shay, D. K., Shu, B., Cox, N. J. & Klimov, A. I. Adamantane resistance among influenza A viruses isolated early during the 2005-2006 influenza season in the United States. J. Am. Med. Assoc. 295, 891-894 (2006). Recently, it has been reported that resistance to rimantadine and amantadine in humans, birds and pigs has reached more than 90%, casting into doubt the continued ability of these drugs alone to satisfy the need for treatment of influenza (Deyde, V. M. et al. Surveillance of resistance to adamantanes among influenza A(H3N2) and A(H1N1) viruses isolated worldwide. J. Infect. Dis. 196, 249-257 (2007)).

### SUMMARY

The present invention provides, among other things, methods for the identification of compounds that are capable of modulating the activity of the influenza A virus. For example, the present methods provide platforms for identifying small molecule inhibitors that target the pathway defined at least in part by any of the residues 27, 30, 31, 34, 37, 41, 44, and 45 of the influenza A M2 protein.

### BRIEF DESCRIPTION OF THE DRAWINGS

FIG. 1 provides several views of the crystal structure of the M2 proton channel from the influenza A virus at>1.65 Angstroms resolution.

FIG. 2 shows the asymmetric structure of the C-terminal His/Trp gate of the M2 transmembrane helix.

FIG. 3 depicts superimpositions of the crystallographic tetramer, which demonstrate conformational differences in the C-terminal gating region of the channel.

FIG. 4 illustrates the minimal mechanism of activation and conductance through the M2 transmembrane channel.

FIG. 5a depicts the electron density of the 2.05 Å structure contoured at 1.2σ, showing a salt bridge between Arg45 and Asp44 on neighboring helices. b) shows how detergent molecules form a bilayer-like environment surrounding the M2TM tetramers (C and O of PEG and n-octyl-β-D glucopyranoside in green and red, respectively, and water molecules in magenta) and help fill voids near the loosely packed C-terminal end of the bundle. c) provides a Bijvoet difference Fourier map calculated with anomalous data collected at a wavelength corresponding to Se-edge, contoured at 5.0σ for chains E, G, H and 4.0σ for F chain.

FIG. 6 depicts how mutating Ser31 (left image) to an Asn sidechain in a low-energy rotamer (right image) produces a model with extensive hydrogen bonding between the Asn carboxamides and a carbonyl-lined hole large enough to accommodate one or more water molecules.

FIG. 7 provides a representation of the high-resolution crystallographic structure of the portion of the M2 transmembrane peptide spanning residues 25-46.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a residue” is a reference to one or more of such residues and equivalents thereof known to those skilled in the art, and so forth. When values are expressed as approximations, by use of the antecedent “about,” it will be understood that the particular value forms another embodiment. As used herein, “about X” (where X is a numerical value) preferably refers to ±10% of the recited value, inclusive. For example, the phrase “about 8” preferably refers to a value of 7.2 to 8.8, inclusive; as another example, the phrase “about 8%” preferably (but not always) refers to a value of 7.2% to 8.8%, inclusive. Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, “2-5”, and the like. In addition, when a list of alternatives is positively provided, such listing can be interpreted to mean that any of the alternatives may be excluded, e.g., by a negative limitation in the claims. For example, when a range of “1 to 5” is recited, the recited range may be construed as including situations whereby any of 1, 2, 3, 4, or 5 are negatively excluded; thus, a recitation of “1 to 5” may be construed as “1 and 3-5, but not 2”, or simply “wherein 2 is not included.”

The disclosures of each patent, patent application, and publication cited or described in this document are hereby incorporated herein by reference, in their entirety.

Provided are methods identifying a compound that modulates the activity of the influenza A virus. The present methods relates to the structure-based design of drugs that target the proton transport pathway defined by the residues that comprise the core of the transmembrane tetramer. This proton transport pathway is defined primarily by residues 27, 30, 31, 34, 37, 41, 44, 45 (the numbering of which is derived from the UDORN strain of influenza A virus), and optionally by crystallographically defined water molecules. Specifically, the present methods comprise comparing spatial models of a plurality of test compounds with a spatial model of the proton transport pathway of the tetrameric M2 transmembrane protein of the influenza A virus; the pathway being defined by at least two residues from among residues 27, 30, 31, 34, 37 or 41, 44, and 45 on one or more subunits of the protein; determining the spatial complementarity of each of the test compounds with the pathway; assessing the ability of the test compounds to bind to the pathway; and, based on the assessed ability of the test compounds to bind to the pathway, determining the compound that modulates the activity of influenza A.

Also disclosed are methods for identifying a test compound that modulates the activity of influenza A comprising comparing a spatial model of the test compound with a spatial model of the proton transport pathway of the tetrameric M2 transmembrane protein of the influenza A virus; the pathway being defined by at least two residues from among residues 27, 30, 31, 34, 37 or 41, 44, and 45 on one or more subunits of the protein; determining the spatial complementarity of the test compound with the pathway; assessing the ability of the test compound to bind to the pathway; and, based on the assessed ability of the test compound to bind the pathway, determining whether the compound modulates the activity of influenza A.

As used herein, the “aqueous pore defined by at least two residues from among residues 27, 30, 31, 34, 37, 41, 44, and 45 on one or more subunits of the protein” refers to the solvent-accessible surface area (Lee-Richards molecular surface) of the pathway defined by at least two residues from among residues 27, 30, 31, 34, 37, 41, 44, and 45 using a probe radius between 0.5 and 2.0 Angstroms. As the M2 transmembrane protein is a tetrameric protein, reference to “at least two residues” can describe two or more residues from a single helical subunit of the tetramer (e.g., Val27 and Ser31 of subunit “A”), the same residue on two different subunits (e.g., Val27 on each of subunits “A” and “B”), at least one residue on one of the subunits of the tetramer and at least one different residue on another of the subunits (e.g., Val27 on subunit “A” and Ser31 on subunit “B”), or any combination thereof, whether such residues are wild-type or mutations, as described more fully below. Thus, the proton transport pathway may be defined, at least in part, by the same residue on two or more of the subunits of the tetrameric protein; by at least two residues on a single subunit of the protein; by at least one residue on one of the subunits and at least one residue on another of the subunits; or, any combination thereof. The proton transport pathway may further comprise any solvent molecules within 5 Å of the sidechains of the listed residues.

In some embodiments, the proton transport pathway may be defined by at least two residues from among residues 27, 30, 31, 34, 37 or 41, 44, and 45 of the M2 protein. The proton transport pathway may also or alternatively be defined by at least three residues from among residues 27, 30, 31, 34, 37, 41, 44, and 45 of the M2 protein. In other embodiments, the proton transport pathway defined by at least two residues from among residues 27, 30, 31, 34, 37, 41, 44, and 45 of the M2 protein may be further defined by the spatial contribution(s) of one or more water molecules within about 5 Angstroms of any of such residues. For example, the proton transport pathway may be further defined by one or more water molecules from the outer cluster, the bridging cluster, or the exit cluster of water molecules as described more fully herein. The residues that contribute to the definition of the proton transport pathway may be wild-type residues (i.e., residues present in wild-type M2 as defined by the transmembrane domain sequence of the A/Udorn/72 strain and the following variants thereof: L26F, V28I, V28A, V28F, V28D, A29V, A29I, A30S, I32V, I32L, I33V, I33L, L36V, L36I, L38F, I39V, I39T, I42M, I42L, I42V, L43I, L43F, L43V, L43T, R45H, R45C), or may result from one or more mutations to the M2 protein. Thus, the spatial model of the proton transport pathway may comprise the transmembrane region of an M2 protein having a mutation at one or more of residues 27, 30, 31, 34, 37, 41, 44, and 45, on any one or more of the subunits of the tetrameric protein. For example, the spatial model of the proton transport pathway may comprise the transmembrane region of an M2 protein having the V27G mutation, the V27I mutation, the V27T mutation, the V27S mutation, or the V27A mutation; may comprise the transmembrane region of an M2 protein having the A30T mutation may comprise the transmembrane region of an M2 protein having the S31A mutation or the S31N mutation; may comprise the transmembrane region of an M2 protein having the G34E mutation or the G34A mutation; may comprise the transmembrane region of an M2 protein having the W41L mutation or the W41Y mutation; may comprise the transmembrane region of an M2 protein having the D44N mutation or the D44H mutation; and/or may comprise the transmembrane region of an M2 protein having the R45K mutation or the R45H mutation. In other embodiments, the spatial model of the proton transport pathway may comprise the transmembrane region of an M2 protein having a mutation that does not prevent the ability of a corresponding M2 protein to transport a proton across a membrane. In other words, the spatial model may correspond to any functional M2 protein that has a mutation at one or more amino acid residues within the M2 protein.

Accordingly, the spatial model of the proton transport pathway may comprise all or part of the transmembrane region of an M2 protein having one or more mutations; a variety of mutations in which pore-forming residues (any of residues 25-46) are changed from the wild-type may readily be modeled from the presently disclosed structure of the influenza A M2 protein. It will be appreciated that spatial models of the proton transport pathway in which one or more mutations are present provide the unique and heretofore unavailable tool for identifying compounds that modulate the activity of influenza A virus having one or more mutations that may arise within the pathogen population. Some of such mutations are known and have specific designations in the literature. For example, the spatial model of the proton transport pathway may comprise all or part of the transmembrane region of an M2 protein having the S31N mutation, or the transmembrane region of an M2 protein having the G34A mutation. In other embodiments, the spatial model of the proton transport pathway may comprise all or part of the transmembrane region of an M2 protein having another mutation, such as, for example, any mutation at residue 27 that results in various amino acids (such as Ile, Ala, Ser, Gly from the wild-type Val); a mutation at residue 30 that results in various amino acids from the wild-type Ala; any mutation at residue 34 that results in various amino acids (e.g., Ala or Glu from the wild-type Gly); or, any mutation within the transmembrane helix that can result in the formation of a hydrophobic residue.

In accordance with the present invention, the spatial models of the test compound(s), the spatial model of the proton transport pathway, or both, are preferably computer-based. Those skilled in the art can readily appreciate numerous computer-based systems for preparing, manipulating, and/or testing spatial models of molecules. The spatial model of the proton transport pathway may comprise at least a portion of the tetrameric four-helix bundle of the M2 protein. In one embodiment, the spatial model of the proton transport pathway comprises all or part of the transmembrane region of a wild-type M2 protein.

The assessment of the ability of the test compound(s) to bind to the proton transport pathway may refer to a comparative test among compounds or between a given compound and a reference standard, such as determining which of the compounds, or which of the compound or the reference standard, displays a higher degree of binding, as measured in accordance with any acceptable parameter for assessing binding. The ability of a compound to bind to the proton transport pathway refers to the binding affinity of the compound for the proton transport pathway defined at least by the residues 27, 30, 31, 34, 37, 41, 44, and 45 of the M2 protein and optionally any solvent molecules within 5 Å of these residues, or the binding affinity of the compound for any other portion of the M2 protein whereby such binding at least partially disrupts the functionality of the pathway defined at least by the residues 27, 30, 31, 34, 37, 41, 44, and 45, and optionally any solvent molecules within 5 Å of these residues. The functionality of the pathway may refer to the physical cooperation in the process of proton conduction by the M2 transmembrane channel.

The determination of whether the compound modulates the activity of influenza A may comprise correlating the assessed ability of the test compound to bind the proton transport pathway to a statistical likelihood that the existence or degree of binding of the compound(s) to the proton transport pathway will affect the proton transport functionality of the M2 transmembrane channel. A finding of a statistical likelihood of the ability of the compound(s) to decrease or forestall the ability of the M2 transmembrane channel to conduct protons may correlate to a positive determination of the ability of the compound(s) to modulate the activity of the influenza A virus.

The present methods for identifying a compound that modulates the activity of influenza A may further comprise testing the compound or compounds which have been found to modulate the activity of influenza A based on the assessed ability of the compound to bind the proton transport pathway. The test of the compound or compounds may comprise an influenza A inhibition assay. The influenza A inhibition assay may be in vivo or in vitro. The prominence of influenza virus research has led to the development of numerous types of influenza inhibition assays with which those skilled in the art are familiar. See, e.g., F G Hayden, K M Cote, and R G Douglas, Jr, Antimicrob Agents Chemother. 1980 May; 17(5): 865-870 (plaque inhibition assay). The inhibition assay may comprise assessing the ability of the compound or compounds to modulate the activity of the M2 transmembrane domain, the full-length A/M2 protein, or fragments of intermediate length (See e.g., C Ma, A L Polishchuk, Y Ohigashi, A L Stouffer, A Schon, E Magavern, X Jing, J D Lear, E Freire, R A Lamb, W F DeGrado, and L H Pinto, Proc Natl Acad Sci USA. 2009 Jul. 28; 106(30):12283-12288. Epub 2009 Jul. 9) in wild type or mutant sequence or may comprise assessing the ability of the compound or compounds to modulate the activity of some other characteristic of the influenza virus.

Although models of the M2 channel have been proposed on the basis of mutagenesis, molecular dynamics, and spectroscopic studies, high-resolution crystallographic or solution NMR structures have not been available. Representing a significant breakthrough, the present invention provides, inter alia, the structure of a peptide spanning the transmembrane helix of M2 (M2TM). In addition, the present invention pertains to the identification and structural characterization of a site on the M2 proton channel defined at least in part by the intermolecular interaction between the residues 27, 30, 31, 34, 37, 41, 44, and 45. The newly-discovered and characterized site is conserved in mutated forms of the M2 proton channel. Advantageously, the present invention includes structure data for the transmembrane portion of M2, including the newly-discovered site, in sufficiently high resolution for enabling drug design.

Like the full-length protein, M2TM associates into a tetrameric four-helix bundle that binds amantadine and conducts protons. Numerous functional variants of M2TM were screened for their ability to form diffraction-quality crystals.

### EXAMPLE 1

### Crystal Structure at >1.65 Angstroms

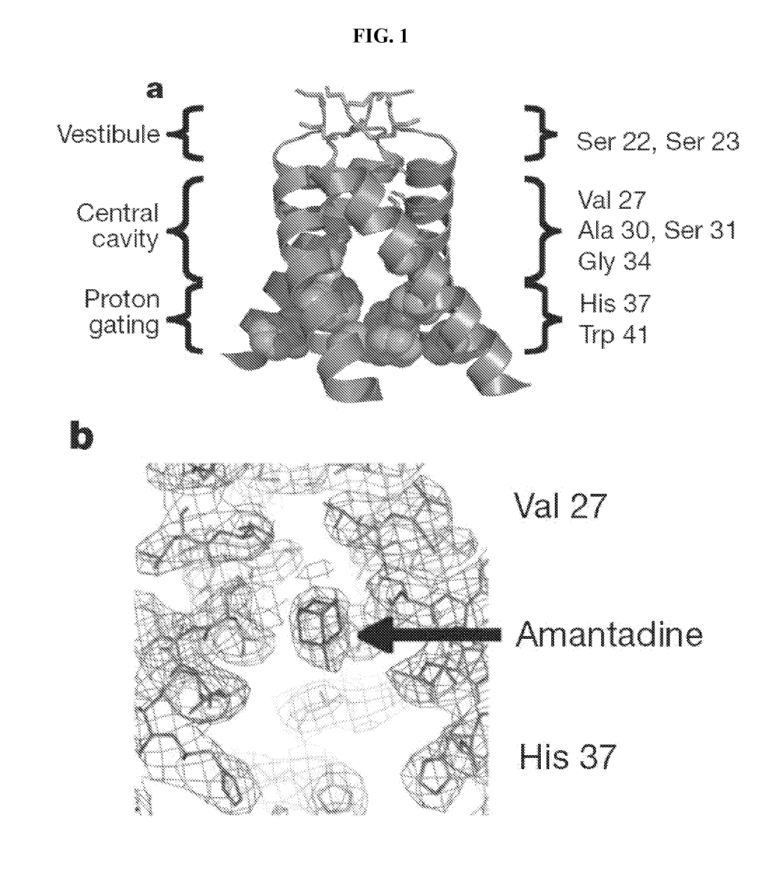

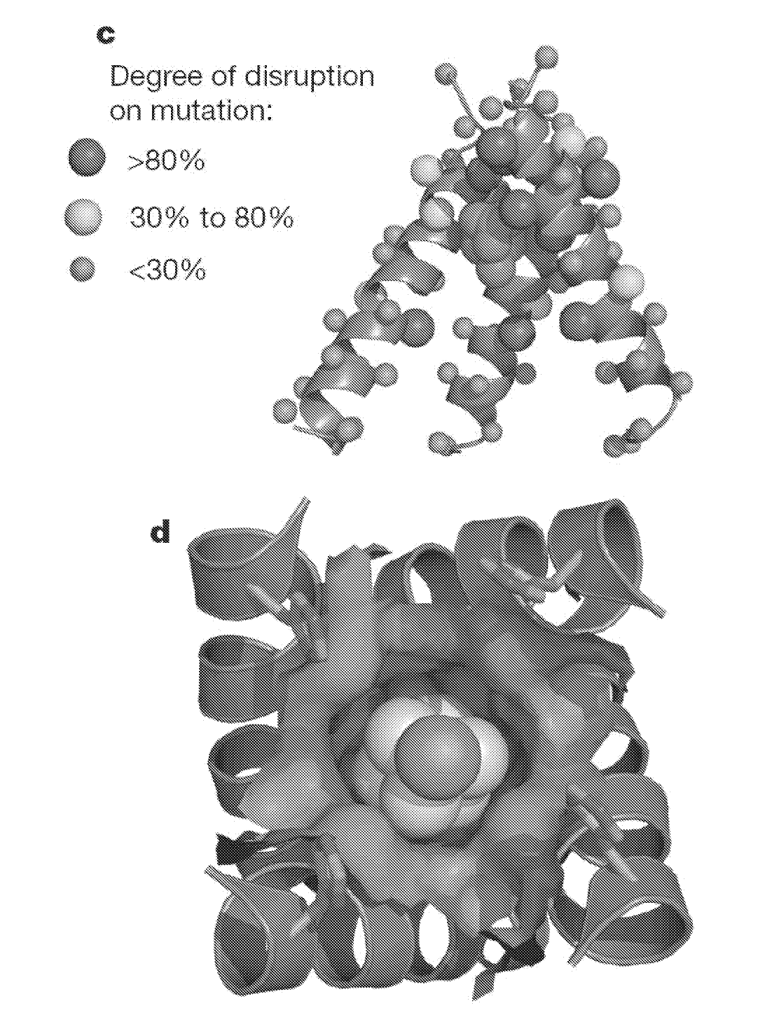

FIG. 1 provides several views of the crystal structure of the M2 proton channel from the influenza A virus at >1.65 Angstroms resolution. In FIG. 1a, the most critical residues identified by site-directed mutagenesis line the pore. Gly 34, His 37 and Trp 41 are shown in space-filling spheres (including carbon atoms of His 37), whereas the side chains of the other critical residues are shown as sticks. FIG. 1b provides an omit map (2Fₒ-F_{c}, contoured at 1s) showing electron density in the amantadine-binding region. In FIG. 1c positions of previously described Cys mutations that disrupt the ability of amantadine to block the channel are shown by balls indicating >80% disruption, 30% to 80% disruption, and no significant disruption, respectively, in full-length M2. Amantadine is shown in the center of the tetramer (the front helix being removed for clarity). FIG. 1d shows the structure of amantadine inside the binding site showing the surface associated with residues Val 27, Ala 30, Ser 31 and Gly 34.

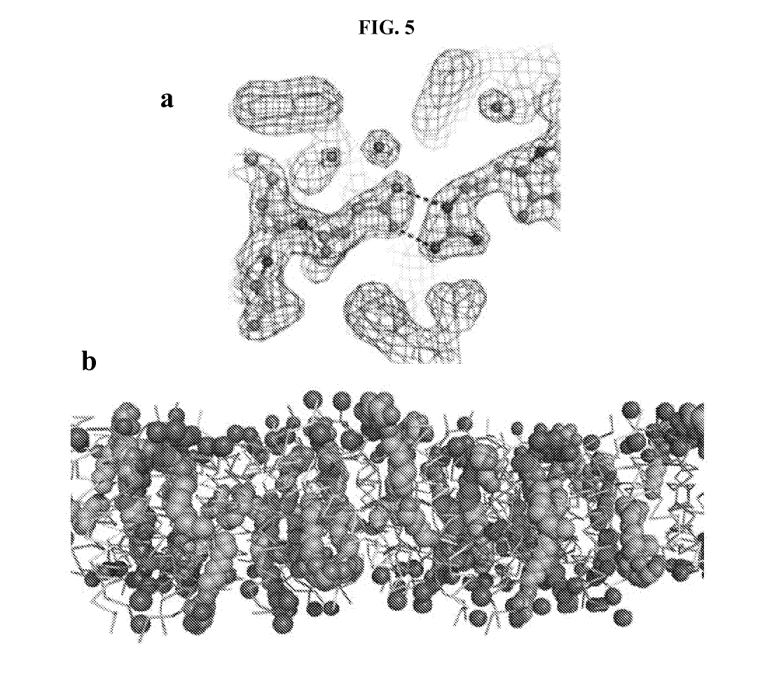

Thus, a crystal form that diffracts to 2.0 Å resolution was obtained from a peptide, in which Ile 33 was changed to selenomethionine (I33-SeMet), at pH 7.3 in the absence of amantadine. The peptide crystallizes with six octyl-b-Dglucopyranoside detergent molecules that form a bilayer-like environment into which M2TM tetramers are embedded (FIG. 5). A second mutant, G34A, was crystallized at a lower pH (pH 5.3) in the presence of amantadine (diffraction limit, 3.5 Å resolution). The two structures are very similar (root mean squared deviation, “r.m.s.d.”, for all atoms is 1.8 Å), with the primary differences lying near the carboxy-terminal region of the helices.

In both structures the tetrameric M2TM helices form a lefthanded, parallel bundle (FIG. 1a) that resembles a conical frustum (a truncated cone), with the narrow amino-terminal end facing the exterior of the viral envelope. Each helix is preceded by a polar, highly conserved sequence, Ser-Ser-Asp (FIG. 1a), four copies of which form a narrow, solvent-filled pore lined by Ser hydroxyls and main-chain carbonyl groups. Protons pass through this extra-viral vestibule en route to the section of the transmembrane pore formed by the four-helix bundle (residues 25-45, FIG. 1a). The N-terminal half of the channel has nearly exact four-fold rotational symmetry; the helices are tilted by 35°±2° with respect to the central axis of the bundle, which is within the range of 30° to 40° observed by solid-state NMR (ssNMR) for both M2TM and a longer fragment of the protein. The pore is most constricted near Val 27; beyond this point it opens to create an aqueous cavity lined by small residues (Ala 30, Ser 31 and Gly 34), reaching a maximal diameter of 9 Å at Gly 34 near the centre of the bilayer. Similar large aqueous cavities are often observed near the centre of channel proteins, where they seem to minimize the thermodynamic cost of bringing a charged ion to the centre of a bilayer. The channel constricts again at the critical pH-gating residues His 37 and Tip 41.

Full-length M2 is inhibited by amantadine with a cooperatively factor of 1.0; the protein binds a single drug molecule per tetramer. The electron-density map from crystals grown in the presence of amantadine shows strong density of the same size and shape as that of this drug molecule (FIG. 1b). The drug is surrounded by residues (including Val 27, Ala 30, Ser 31 and Gly 34) that are mutated in clinical isolates of amantadine-resistant viruses. The same hotspot for amantadine resistance was pinpointed in positional scanning mutagenesis of the full-length protein (FIG. 1b, c). Furthermore, residues that can be mutated without affecting amantadine inhibition lie more distal to the drug-binding site at membrane-accessible and C-terminal locations (including Leu 38 to Asp 44). The structure is in agreement with electrophysiological studies of full-length M2 that showed that the rate of channel block is approximately 105-fold slower than that expected for diffusion of a small molecule into a channel with a large extracellular opening. The highly restricted vestibule helps to explain the slow kinetics of entry of the drug, which might enter the site by means of rare conformational changes or laterally from the bilayer phase.

The drug-binding site is nearly identical (r.m.s.d=0.4 Å over all atoms of Val 27, Ala 30, Ser 31 and Gly 34) between the amantadine-bound (low pH) and the drug-free (high pH) crystal structures; this is consistent with amantadine's known ability to inhibit between pH 5.0 and pH 8.0. Given the resolution of the structure, two orientations of the drug are possible: the amine group could either point towards the viral exterior or point inward, where it would be hydrated in the aqueous pore (FIG. 1b). The drug fits the density better in the inward orientation with its large, apolar group snugly fit into the N-terminal end of the aqueous cavity (FIG. 1b, c). The polar end of the drug projects towards, but does not directly contact, His 37. This binding mode is consistent with the fact that if the amino group of amantadine is substituted with structurally diverse bulky secondary alkylamines, inhibitory activity is retained. Long-range interactions between the ammonium group and His 37 might also account for shifts in this residue's pKa that accompany the binding of the drug.

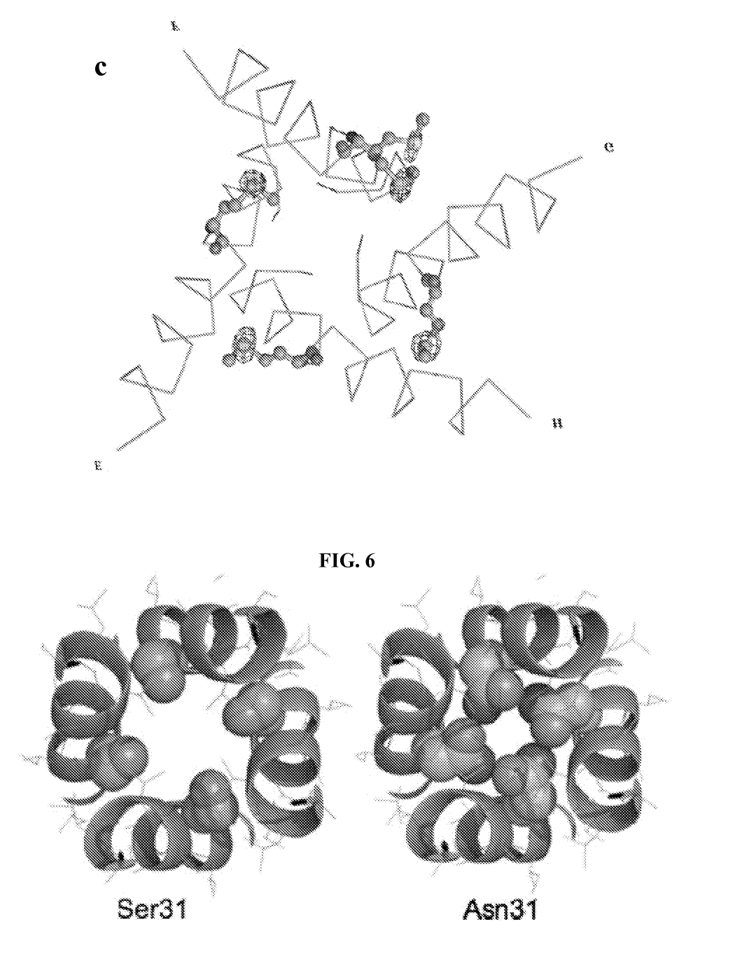

The recent, marked rise in amantadine resistance has been associated with a single mutation, S31N. In the 2005-2006 flu season, resistance reached more than 90%, and 99.9% of the resistant viruses collected worldwide (1,059 of 1,060) had the S31N mutation. Asn can be modeled in a low-energy rotamer at position 31 of the crystal structure, resulting in extensive hydrogen bonding between the Asn carboxamides (FIG. 6). The side chains form a carbonyl-lined hole that can accommodate one or more water molecules, explaining the retention of proton-channel activity in this mutant. This mutation also constricts the size and increases the polarity of the amantadine-binding site—features that should interfere with the binding of the large hydrophobic adamantane group. Furthermore, Ser-to-Asn mutations stabilize transmembrane helix-helix association. Thus, the S31N mutation seems to be particularly fit in terms of its ability to allow proper insertion, assembly and function of M2 in membranes, while escaping inhibition by amantadine. Interestingly, although S31N constricts the amantadine-binding site in the N-terminal half of the transmembrane pore, this mutation does not fill the aqueous pore defined by His 37 and Trp 41—functionally essential residues conserved in all influenza A and B viruses. New drugs that bind this region could inhibit both Ser 31 and Asn 31 variants of M2, and they should also be less susceptible to the development of new resistance.

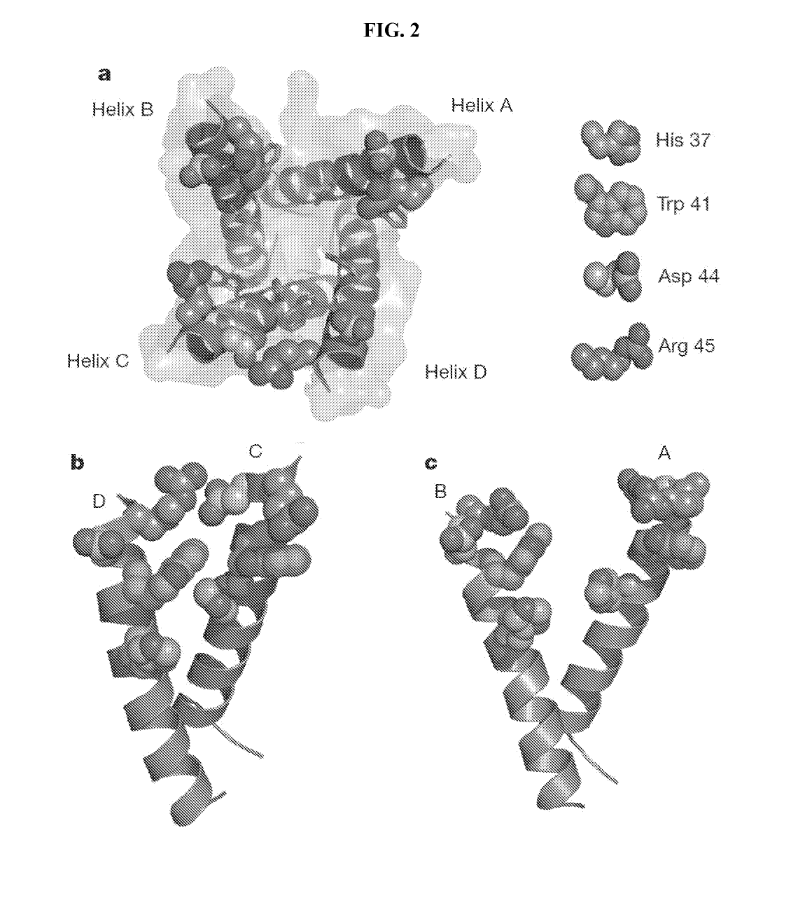

The crystal structures at neutral and low pH provide insight into the role of His 37 and Trp 41 in the conduction of protons. These residues lie near the channel exit within the wide end of the bundle. In the low-pH structure, all four helices are straight and diverge from a point where the helical axes most closely approach the central fourfold symmetry axis, which occurs in the vicinity of Val 27. This divergence creates to a large opening near His 37 and Trp 41. However, the pore of the neutral-pH form is smaller and less symmetric; helix D has a gradual bend of 15° near Gly 34, allowing Trp 41_{D} (the subscript refers to helical subunit D) to interact with His 37C in an edge-on aromatic interaction (FIG. 2a, b) previously predicted from spectroscopic and ssNMR measurements. This conformation is further stabilized by an interhelical salt bridge between Arg 45_{D} and Asp 44_{C} (FIG. 2a, b). In contrast, the A and B helices are more similar to the symmetrical low-pH form. They are nearly completely straight and consequently diverge too far to allow interhelical His-Trp and Arg-Asp interactions (FIG. 2a, c).

The finding of multiple conformations at the gating end of the channel is consistent with the known pH-dependent dynamic and functional properties of M2. The neutral form was crystallized at pH 7.3, at which the four His 37 residues should be in a mixed protonation state on the basis of their known pKa values in M2TM (8.2, 8.2, 6.3 and <5). The pH-dependent structural variability seen between the low-pH and neutral structures is consistent with disulphide crosslinking studies in the full-length protein that demonstrated a selective increase in C-terminal contacts at a neutral pH. Also, spectroscopic measurements of M2TM in phospholipid bilayers have revealed that the helices can be straight or bent near Gly 34. The curve in helix D occurs in the vicinity of Gly 34, which is one turn apart from a pair of small side chains, Ala 30 and Ser 31. Similar sequences are believed to mediate bending of transmembrane helices during the gating of potassium channels. Interestingly, the low-pH form was solved using the G34A mutation; if this region is indeed part of a hinge, this mutation might help to stabilize an open conformation with straight rather than curved helices.

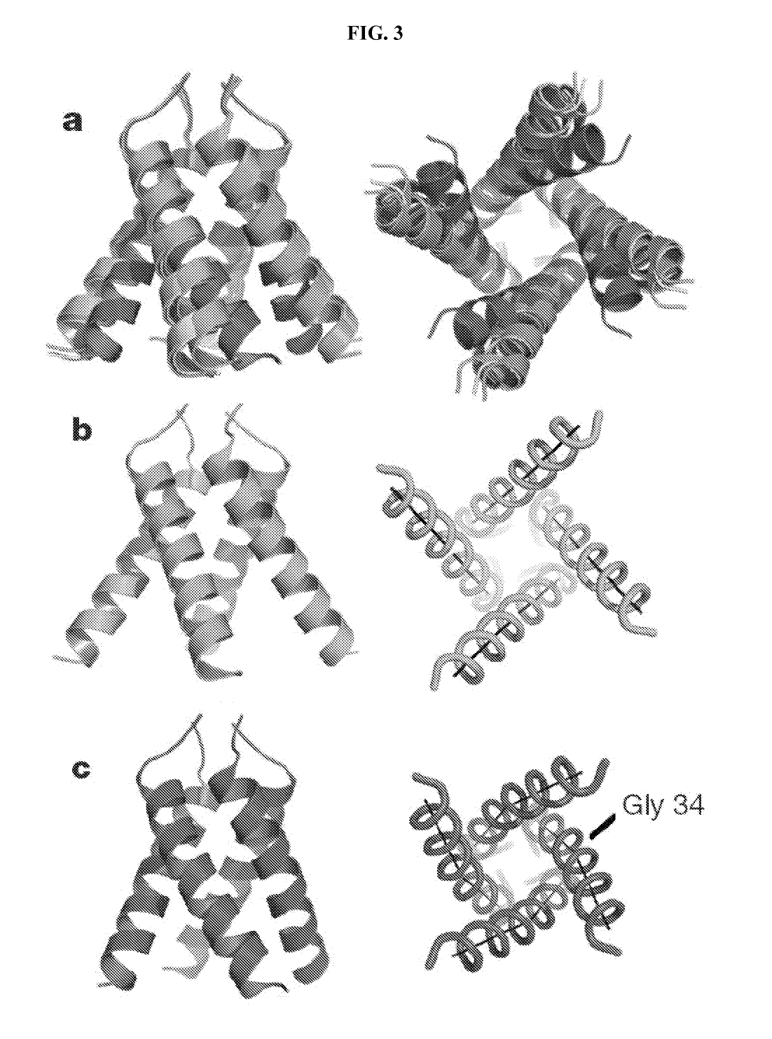

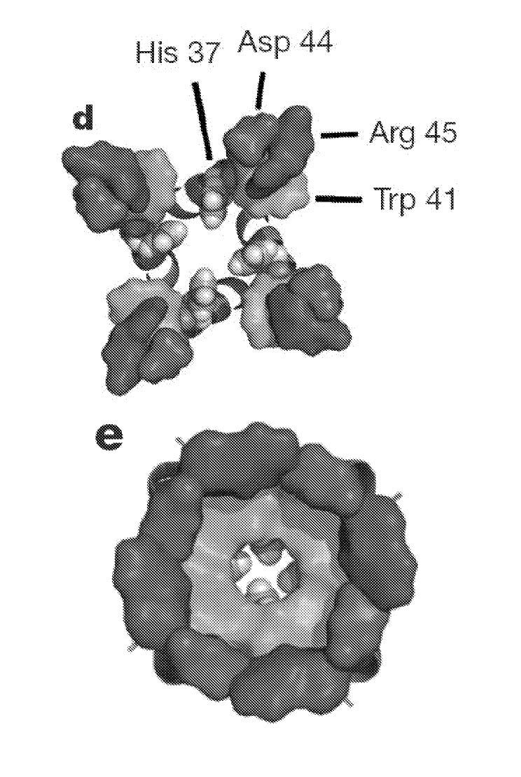

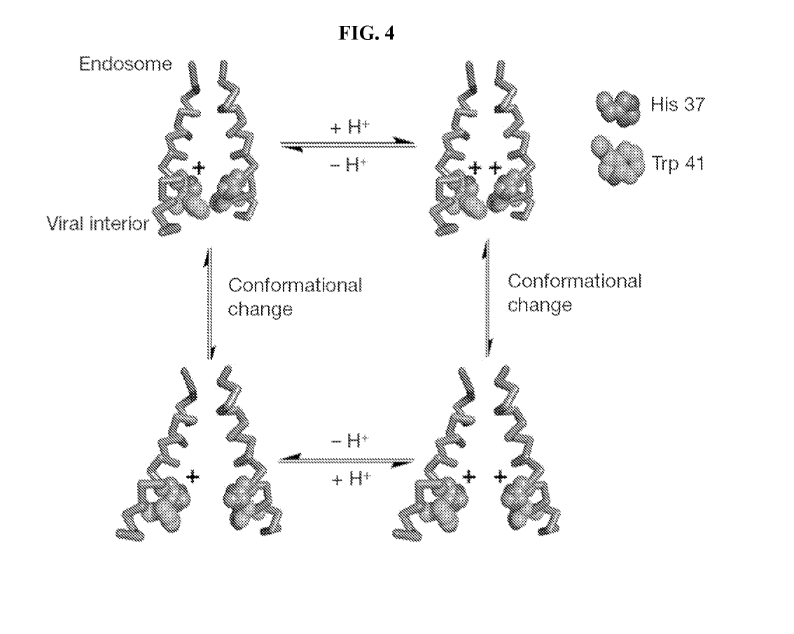

To explore the functional implications of the conformational differences in the subunits of the neutral-pH form, chains A, B, C and D of M2TM were superimposed onto B, C, D and A, respectively, and this procedure was repeated for each of the remaining cyclic permutations (FIG. 3a). The resulting family of four tetramers superpose extremely well at the N terminus of the bundle, but become increasingly divergent at the C terminus. The four copies of helix A form a bundle, designated A4, in which the straight helices diverge near the C terminus as in the low-pH structure (FIG. 3b, r.m.s.d.=1.5 Å over all atoms). At the other extreme, the D4 bundle, which is almost completely closed at the C terminus of the bundle, is similar to a coiled-coil (FIG. 3c-e). The geometry was optimized by energy minimization using 1,000 cycles of XPLOR-NIH (see Methods, below), which resulted in only small changes in the backbone (0.8 Å and 1.4 Å r.m.s.d. for A4 and D4, respectively) and retained the native rotameric states of the side chains. The A4 and D4 models suggest a minimal mechanism for the pH-dependent activation of the channel (FIG. 4). At high-pH values, the channel would be mostly in a D4-like ‘closed’ state, whereas a decrease in pH would trigger electrostatic repulsions between multiple protonated His residues to a more open A4-like state with improved hydration of the charged His side chains. In FIG. 4, two helices of the tetramer and one protonation event are shown for simplicity.

This mechanism is in agreement with the effects of mutations on the pH-dependent activation of the channel. The His_{C}-Trp_{D} and Arg_{D}-Asp_{C} interactions seen in the helix C-D interface of the crystal structure (FIG. 2b) and at each interface in the D4 bundle (FIG. 3e) seem to help to stabilize the closed conformation. Mutating Asp 44 to Asn caused a large increase in the activity of M2; presumably, this mutation shifts the equilibrium to the open conformation by disrupting the Asp-Arg salt bridge. Furthermore, Trp 41, which helps trap protons within an acidified virus, is found to block access of His 37 from the side of the D4 tetramer facing the inside of the virus (FIG. 3e).

The structure-based model for the open state (A4 tetramer) is also consistent with the electrophysiological properties of M2. Although M2 is considered to be a channel, it has a very low maximal conductance rate of less than 10⁴ protons per second—much lower than that of typical ion channels, which usually have maximal rates of 10⁵ to 10⁷ ions per second. The slow rate of M2 is consistent with the restricted channel vestibule and argues against a highly populated open state with either a very large pore or an uninterrupted organized ‘wire’ of water molecules. Furthermore, His 37 lies about two-thirds of the way through the transmembrane electrical field, which also argues against a large (>10 Å) uninterrupted pore leading from the outside of the virus to His 37 that would place this residue near the beginning of the transmembrane potential gradient. Instead, the highly restricted N-terminal vestibule might contribute to proton selectivity. N-terminal motions would allow protons to penetrate into the aqueous pore by means of transient hydrogen-bonded chains of water molecules, whereas it might be very difficult for hydrated sodium or potassium to penetrate this region. Indeed, amantadine-sensitive variants in which Val 27, which lines the most constricted point in the pore, is mutated to Ser, Thr or Cys have increased conductance and/or compromised ion selectivity.

Earlier predictions are in reasonable accord with the crystallographic structures in terms of many of the overall features of the channel. The r.m.s.d. between experimentally restrained models on the basis of site-directed mutagenesis and conformational searching is 3 Å to 4 Å, and these models accurately predicted the location of critical residues in the pore. The helix-crossing angles of the crystallographic structures are within the range of angles detected by ssNMR studies of M2TM in buffered solution. Early attempts to define models on the basis of restraints from ssNMR were complicated by conformational averaging. However, a recent study of the amantadine complex at high pH showed a conformation for the helical monomer in excellent agreement with the bent helix D of the crystal structure (15° versus 11° by ssNMR). A model based on these ssNMR angular restraints is consistent with the overall features of the crystallographic structures and the D4 model (all-atom r.m.s.d.=˜3.0 to ˜3.3 Å).

Methods. Solid-phase peptide synthesis. The sequence of wild-type M2TM is SSDPLVVAASIIGILHLILWILDRL. Single-site variants of M2TM were chemically synthesized using solid-phase peptide synthesis with 9-fluorenylmethoxycarbonyl (Fmoc) chemistry. Cleavage from resin and deprotection of amino acid protecting groups was carried out in a mixture of trifluoroacetic acid (TFA)/triisopropyl silane/H₂O (90:5:5 v/v) at room temperature (25° C.) under nitrogen for four hours. Purification proceeded by reverse-phase high-performance liquid chromatography (HPLC) using a preparative C4 column (Vydac) and a linear gradient of buffer A (99.9% H₂O and 0.1% trifluoroacetic acid) and buffer B (60% acetonitrile, 30% isopropanol, 9.9% H₂O and 0.1% trifluoroacetic acid). The purity and molecular mass of M2TM peptides were assessed by analytical HPLC on a C4 column and matrix-assisted laser desorption/ionization-time of flight (MALDI-TOF) mass spectrometry.

Crystallization. Crystals of selenomethionine-labelled M2TM segment (133-SeMet) were grown by the hanging-drop vapour diffusion method by mixing equal volumes of protein solution and well solution. Initial attempts at crystallization showed that the protein crystallized from approximately 20% PEG MME (polyethylene glycol monomethyl ether) at a variety of pH values. Additive screens showed that xylitol and MgCl₂ improved the quality of the crystals. These were combined to provide crystallization conditions consisting of equal volumes of: solution A (1 mM protein, 50 mM octyl-β-D-glucopyranoside and 5% w/v xylitol) and solution B (50 mM Tris-HCl, 0.5 M MgCl₂, 21% PEG 350 MME). The final pH after addition of all components was 7.3.

A similar additive screen showed that Ni(II) also improved diffraction. M2TM-segment G34A mutant crystals were obtained using the hanging-drop vapor diffusion method by mixing equal volumes of the protein solution (0.8 mM protein, 0.6 mM amantadine, 32 mM octyl-β-D-glucopyranoside and 5% w/v xylitol) with pH 8 reservoir solution (50 mM Tris-HCl, 0.5 mM MgCl₂, 30 mM NiCl₂and 22% PEG 350 MME). It was found that the Tris buffer interacted with NiCl₂ and/or other components of the buffer, resulting in release of protons and reducing the pH of the solution to 5.3.

X-ray diffraction data collection and processing. Synchrotron data for the M2TM I33-SeMet crystal were collected on beamline X12-C at the National Synchrotron Light Source, Brookhaven National Laboratory. Three multiwavelength anomalous diffraction data sets were collected. During data collection, the crystal was cryo-cooled to 100° K. The data sets were processed using DENZO and SCALEPACK. The crystals were found to belong to space group P2₁, and the diffraction data were processed to a maximum resolution of 2.0 Å. The crystallographic details are shown in Table 1, below.

TABLE 1

| | M2TM I33SeMet | | M2TM G34A | |
|---|---|---|---|---|
| Data collection | | | | |
| Space group | P2₁ | | P2₁2₁2 | |
| Cell dimensions | | | | |
| a, b, c (Å) | 38.75, 56.56, 56.01 | | 60.40, 57.83, 38.11 | |
| α, β, γ (°) | 96, 103.5, 90 | | 90, 90, 96 | |
| Resolution (Å) | 50-2.0 | (2.05-2.0)*^{, a} | 15-3.5 | (3.61-3.5)* |
| R_{merge} (%) | 6.2 | (30.5) | 10.5 | (36.2) |
| I/σ(I) | 22.4 | (1.9) | 12.3 | (3.1) |
| Completeness (%) | 97.4 | (75.1) | 99.5 | (100) |
| Redundancy | 3.4 | (2.0) | | |
| Refinement | | | | |

| | | |
|---|---|---|
| Resolution (Å) | 20-2.05 | 15-3.5 |
| No. reflections | 14619 | 1530 |
| R_{work}/R_{free} (%/%) | 21.9/26.9 | 29.0/31.8 |
| No. atoms | | |
| Protein | 1561 | 772 |
| Ligand/ion | 164 | 11 |
| Water | 39 | |
| B-factors (Å²) | | |
| Protein | 27.9 | 39.6 |
| Ligand/ion | 58.6 | 51.1 |
| Water | 46.1 | |
| R.M.S deviations | | |
| Bond lengths (Å) | 0.011 | 0.029 |
| Bond angles (°) | 1.348 | 2.185 |

*Highest resolution shell is shown in parenthesis.
^{a}Although the data were processed to 2.0 Å, the maximum resolution used for refinement was up to 2.05 Å, with completeness (%) = 94.2, R_{merge} (%) = 25.5, I/σ(I) = 2.5 and redundancy = 2.4 in the last resolution shell 2.10-2.05 Å.

Crystals of M2TM(G34A) were cryo-cooled to 100° K, and the diffraction data were collected on a diffractometer equipped with Cu(Kα) radiation and a MAR image plate detector (345 mm) at the home source. The data sets were integrated with DENZO and were scaled with SCALEPACK. The crystal belongs to the space group P2₁2₁2 and was diffracted to a maximum resolution of 3.5 Å(Table 1).

Structure determination and refinement. For the M2TM(G34A)-amantadine complex, the Matthews number (2.96 Å³ Da⁻¹) suggested four molecules in the asymmetric unit with a solvent content of 58.6 for the G34A data. The structure solution was obtained by the molecular replacement method using a polyalanine α-helix of 23 residues in length as a search probe. Molecular-replacement calculations were performed using the program PHASER and using the data in the resolution range 13.0-4.0 Å.

The initial model obtained was subjected to iterative model building and refinement. Initially, a few cycles of model refinement were done using CCP4 suite REFMAC and then by CNS. When all the side chains were included during the model building, a few cycles of simulated annealing refinement were carried out using the program suite CNS38. At the later stages of refinement, REFMAC was used. Table 1 gives the statistics of the final structure. The model building was carried out using COOT.

In the M2TM I33-SeMet crystal there are eight molecules in the asymmetric unit, as estimated by Matthews number (2.65 Å³ Da⁻¹). Preliminary analyses using XPREP on three wavelength data sets suggested the presence of anomalous signals up to 3.6 Å resolution. Attempts to obtain experimental phases from anomalous signals by MAD as well as SAD approaches were not successful. Molecular replacement using a single α-helix as a search model was also not successful, but succeeded when the initial solution for the M2TM(G34A) structure was used as the search model. Molecular replacement was carried out using PHASER on peak data sets in the resolution range 15.0-4.0 Å.

Iterative refinement and model building were carried out on the initial model. During the initial stages, all side chains could be traced in the electron density map. As the refinement progressed, detergent (octyl-β-D-glucopyranoside) and PEG 350 MME molecules were located in the electron density map. The refinement was facilitated by applying non-crystallographic restraints on the two tetramers in the asymmetric unit. Release of the noncrystallographic symmetry restraints failed to improve the refinement statistics, so the two tetramers were considered to be identical. Table 1 shows the refinement and final model statistics. The refinement was carried out using CCP4 program suite REFMAC and all the model building was done using COOT.

Computational studies. Each C₄-symmetric tetramer (designated A4, B4, C4 or D4, depending on which monomer was used) was constructed by optimally superimposing the Cα atoms of residues 22-32 onto a cyclically permuted tetramer. For example, to generate the A4 tetramer, Cα atoms from residues 22-32 from chains (A, B, C, D) were optimally superimposed onto Cα atoms from residues 22-32 from chains (A, B, C, D), (B, C, D, A), (C, D, A, B) and (D, A, B, C). Chain A from each optimally superimposed structure was then re-labeled (A, B, C and D, respectively) and used to construct the A4 tetramer. The same procedure was used to construct the B4, C4 and D4 tetramers.

Energy minimization was carried out using the XPLOR-NIH (version 1.1.2) implementation of the CHARMM22 force field. Histidines on chains A and C were doubly protonated and histidines on chains B and D were singly protonated on the ε nitrogen. Hydrogen atoms were added to each structure using the ‘BUILD’ routine followed by 1,000 steps of Powell minimization using a distance-dependent dielectric constant with ε=4.

The kink angle was defined as the angle of deviation between best-fit lines through the N-terminal and C-terminal halves of a helix. For each helix of the tetramer, the N-terminal region consisted of residues 25-34 and the C-terminal region consisted of residues 35-46. Using Cα atoms only, best-fit lines through each region of the helix were constructed using a modified version of an existing algorithm.

### EXAMPLE 2

### High Resolution Structure of M2 Transmembrane Region

A high resolution X-ray crystallography structure of the transmembrane region (amino acids 25-46) of the M2 protein Gly34Ala variant (referred to as M2TM′ G34A) was obtained and a medium-resolution data set on the WT variant of this construct (Gly34) was also obtained and refined. The crystal asymmetric unit comprises a single channel defined by the self-assembly of four transmembrane helices, as described above. The crystal structure is highly symmetric and reflects an activated form when induced by low-pH conditions, or “resting” conformation under high-pH conditions. During the structure refinement stages, fifteen water molecules were traced inside the channel. These systematically coordinated water molecules can contribute to a complete understanding of the channel conduction mechanism and enable the design of new drugs. The high diffraction quality crystals of the transmembrane construct were grown in high-pH medium.

Atomic coordinates for the high resolution crystal structure of M2TM′ G34A and the medium resolution model of M2TM′ G34 described above are provided below in Tables 2 and 3, respectively.

TABLE 2
M2TM′ G34A Coordinates

| HEADER | --- | XX-XXX-XX xxxx |
|---|---|---|
| COMPND | --- | |
| REMARK | 3 | |

| REMARK | 3 | REFINEMENT. | |
|---|---|---|---|
| REMARK | 3 | PROGRAM : REFMAC 5.2.0019 | |
| REMARK | 3 | AUTHORS : MURSHUDOV, VAGIN, DODSON | |
| REMARK | 3 | | |
| REMARK | 3 | REFINEMENT TARGET : MAXIMUM LIKELIHOOD | |
| REMARK | 3 | | |
| REMARK | 3 | DATA USED IN REFINEMENT. | |
| REMARK | 3 | RESOLUTION RANGE HIGH | (ANGSTROMS): 1.65 |
| REMARK | 3 | RESOLUTION RANGE LOW | (ANGSTROMS): 31.53 |

| REMARK | 3 | DATA CUTOFF (SIGMA(F)): NONE | |
|---|---|---|---|
| REMARK | 3 | COMPLETENESS FOR RANGE (%): 100.00 | |
| REMARK | 3 | NUMBER OF REFLECTIONS : 10998 | |
| REMARK | 3 | | |
| REMARK | 3 | FIT TO DATA USED IN REFINEMENT. | |
| REMARK | 3 | CROSS-VALIDATION METHOD : THROUGHOUT | |
| REMARK | 3 | FREE R VALUE TEST SET SELECTION: RANDOM | |
| REMARK | 3 | R VALUE (WORKING + TEST SET): 0.21005 | |
| REMARK | 3 | R VALUE (WORKING SET): 0.20907 | |
| REMARK | 3 | FREE R VALUE : 0.22963 | |
| REMARK | 3 | FREE R VALUE TEST SET SIZE (%): 4.8 | |
| REMARK | 3 | FREE R VALUE TEST SET COUNT : 554 | |
| REMARK | 3 | | |
| REMARK | 3 | FIT IN THE HIGHEST RESOLUTION BIN. | |
| REMARK | 3 | TOTAL NUMBER OF BINS USED | : 20 |
| REMARK | 3 | BIN RESOLUTION RANGE HIGH | : 1.650 |
| REMARK | 3 | BIN RESOLUTION RANGE LOW | : 1.693 |

| | | |
|---|---|---|
| REMARK | 3 | REFLECTION IN BIN (WORKING SET): 750 |
| REMARK | 3 | BIN COMPLETENESS (WORKING + TEST) (%): 100.00 |
| REMARK | 3 | BIN R VALUE (WORKING SET): 0.272 |
| REMARK | 3 | BIN FREE R VALUE SET COUNT : 38 |
| REMARK | 3 | BIN FREE R VALUE : 0.295 |
| REMARK | 3 | |
| REMARK | 3 | NUMBER OF NON-HYDROGEN ATOMS USED IN REFINEMENT. |
| REMARK | 3 | ALL ATOMS : 792 |
| REMARK | 3 | |
| REMARK | 3 | B VALUES. |
| REMARK | 3 | FROM WILSON PLOT (A**2): NULL |
| REMARK | 3 | MEAN B VALUE (OVERALL, A**2): 15.084 |
| REMARK | 3 | OVERALL ANISOTROPIC B VALUE. |
| REMARK | 3 | B11 (A**2): −0.15 |
| REMARK | 3 | B22 (A**2): 0.14 |
| REMARK | 3 | B33 (A**2): 0.02 |
| REMARK | 3 | B12 (A**2): 0.00 |
| REMARK | 3 | B13 (A**2): 0.00 |
| REMARK | 3 | B23 (A**2): 0.00 |
| REMARK | 3 | |
| REMARK | 3 | ESTIMATED OVERALL COORDINATE ERROR. |
| REMARK | 3 | ESU BASED ON R VALUE (A): 0.112 |
| REMARK | 3 | ESU BASED ON FREE R VALUE (A): 0.103 |
| REMARK | 3 | ESU BASED ON MAXIMUM LIKELIHOOD (A): 0.063 |
| REMARK | 3 | ESU FOR B VALUES BASED ON MAXIMUM LIKELIHOOD (A**2): 1.792 |
| REMARK | 3 | |
| REMARK | 3 | CORRELATION COEFFICIENTS. |
| REMARK | 3 | CORRELATION COEFFICIENT FO-FC : 0.937 |
| REMARK | 3 | CORRELATION COEFFICIENT FO-FC FREE : 0.920 |
| REMARK | 3 | |
| REMARK | 3 | RMS DEVIATIONS FROM IDEAL VALUES COUNT RMS WEIGHT |
| REMARK | 3 | BOND LENGTHS REFINED ATOMS (A): 778 ; 0.006 ; 0.022 |
| REMARK | 3 | BOND ANGLES REFINED ATOMS (DEGREES): 1037 ; 0.842 ; 2.054 |
| REMARK | 3 | TORSION ANGLES, PERIOD 1 (DEGREES): 84 ; 2.725 ; 5.000 |
| REMARK | 3 | TORSION ANGLES, PERIOD 2 (DEGREES): 16 ;53.338 ;20.000 |
| REMARK | 3 | TORSION ANGLES, PERIOD 3 (DEGREES): 124 ;10.488 ;15.000 |
| REMARK | 3 | TORSION ANGLES, PERIOD 4 (DEGREES): 4 ;19.285 ;15.000 |
| REMARK | 3 | CHIRAL-CENTER RESTRAINTS (A**3): 143 ; 0.052 ; 0.200 |
| REMARK | 3 | GENERAL PLANES REFINED ATOMS (A): 468 ; 0.003 ; 0.020 |
| REMARK | 3 | NON-BONDED CONTACTS REFINED ATOMS (A): 392 ; 0.195 ; 0.200 |
| REMARK | 3 | NON-BONDED TORSION REFINED ATOMS (A) : 564 ; 0.309 ; 0.200 |
| REMARK | 3 | H-BOND (X...Y) REFINED ATOMS (A): 22 ; 0.050 ; 0.200 |
| REMARK | 3 | SYMMETRY VDW REFINED ATOMS (A): 23 ; 0.134 ; 0.200 |
| REMARK | 3 | |
| REMARK | 3 | ISOTROPIC THERMAL FACTOR RESTRAINTS. COUNT RMS WEIGHT |
| REMARK | 3 | MAIN-CHAIN BOND REFINED ATOMS (A**2): 464 ; 0.699 ; 1.500 |
| REMARK | 3 | MAIN-CHAIN ANGLE REFINED ATOMS (A**2): 716 ; 0.852 ; 2.000 |
| REMARK | 3 | SIDE-CHAIN BOND REFINED ATOMS (A**2): 378 ; 1.966 ; 3.000 |
| REMARK | 3 | SIDE-CHAIN ANGLE REFINED ATOMS (A**2): 321 ; 2.111 ; 4.500 |
| REMARK | 3 | |
| REMARK | 3 | NCS RESTRAINTS STATISTICS |
| REMARK | 3 | NUMBER OF NCS GROUPS : NULL |
| REMARK | 3 | |
| REMARK | 3 | |
| REMARK | 3 | TLS DETAILS |
| REMARK | 3 | NUMBER OF TLS GROUPS : NULL |
| REMARK | 3 | |
| REMARK | 3 | |
| REMARK | 3 | BULK SOLVENT MODELLING. |
| REMARK | 3 | METHOD USED: MASK |
| REMARK | 3 | PARAMETERS FOR MASK CALCULATION |
| REMARK | 3 | VDW PROBE RADIUS : 1.20 |
| REMARK | 3 | ION PROBE RADIUS : 0.80 |
| REMARK | 3 | SHRINKAGE RADIUS : 0.80 |
| REMARK | 3 | |
| REMARK | 3 | OTHER REFINEMENT REMARKS: NULL |
| REMARK | 3 | |

| | | | | | |
|---|---|---|---|---|---|
| LINK | C7 | BRB C 1 | N | PRO C 25 | BRB-PRO |
| LINK | C7 | BRB D 1 | N | PRO D 25 | BRB-PRO |
| LINK | C7 | BRB E 1 | N | PRO E 25 | BRB-PRO |
| LINK | C7 | BRB F 1 | N | PRO F 25 | BRB-PRO |
| LINK | N | NH2 C 47 | C | LEU C 46 | NH2-LEU |
| LINK | N | NH2 D 47 | C | LEU D 46 | NH2-LEU |
| LINK | N | NH2 E 47 | C | LEU E 46 | NH2-LEU |
| LINK | N | NH2 F 47 | C | LEU F 46 | NH2-LEU |

| CRYST1 | 48.665 79.091 48.559 90.00 90.00 90.00 C 2 2 21 | | | | | | | | | | |
|---|---|---|---|---|---|---|---|---|---|---|---|
| SCALE1 | 0.020549 0.000000 0.000000 0.00000 | | | | | | | | | | |
| SCALE2 | 0.000000 0.012644 0.000000 0.00000 | | | | | | | | | | |
| SCALE3 | 0.000000 0.000000 0.020594 0.00000 | | | | | | | | | | |
| ATOM | 1 | BR4 | BRB | C | 1 | −7.404 | 0.079 | 4.848 | 1.00 | 34.21 | BR |
| ATOM | 2 | C4 | BRB | C | 1 | −6.353 | −1.432 | 5.262 | 1.00 | 30.00 | C |
| ATOM | 3 | C3 | BRB | C | 1 | −6.129 | −1.753 | 6.599 | 1.00 | 30.40 | C |
| ATOM | 4 | C2 | BRB | C | 1 | −5.359 | −2.867 | 6.913 | 1.00 | 30.33 | C |
| ATOM | 5 | C5 | BRB | C | 1 | −5.816 | −2.207 | 4.237 | 1.00 | 30.27 | C |
| ATOM | 6 | C6 | BRB | C | 1 | −5.041 | −3.319 | 4.553 | 1.00 | 29.99 | C |
| ATOM | 7 | C1 | BRB | C | 1 | −4.813 | −3.644 | 5.892 | 1.00 | 29.58 | C |
| ATOM | 8 | C7 | BRB | C | 1 | −3.971 | −4.849 | 6.238 | 1.00 | 25.79 | C |
| ATOM | 9 | O1 | BRB | C | 1 | −4.236 | −5.848 | 5.244 | 1.00 | 24.49 | O |
| ATOM | 10 | N | PRO | C | 25 | −3.217 | −5.075 | 7.238 | 1.00 | 21.35 | N |
| ATOM | 11 | CA | PRO | C | 25 | −2.422 | −6.296 | 7.359 | 1.00 | 19.91 | C |
| ATOM | 12 | CB | PRO | C | 25 | −1.622 | −6.058 | 8.639 | 1.00 | 20.36 | C |
| ATOM | 13 | CG | PRO | C | 25 | −1.426 | −4.588 | 8.662 | 1.00 | 20.76 | C |
| ATOM | 14 | CD | PRO | C | 25 | −2.708 | −4.012 | 8.128 | 1.00 | 21.11 | C |
| ATOM | 15 | C | PRO | C | 25 | −3.264 | −7.567 | 7.486 | 1.00 | 18.88 | C |
| ATOM | 16 | O | PRO | C | 25 | −2.827 | −8.629 | 7.047 | 1.00 | 18.53 | O |
| ATOM | 17 | N | LEU | C | 26 | −4.455 | −7.457 | 8.073 | 1.00 | 17.33 | N |
| ATOM | 18 | CA | LEU | C | 26 | −5.360 | −8.605 | 8.177 | 1.00 | 16.20 | C |
| ATOM | 19 | CB | LEU | C | 26 | −6.612 | −8.249 | 8.991 | 1.00 | 16.43 | C |
| ATOM | 20 | CG | LEU | C | 26 | −7.757 | −9.272 | 9.032 | 1.00 | 16.41 | C |
| ATOM | 21 | CD1 | LEU | C | 26 | −8.991 | −8.701 | 9.723 | 1.00 | 16.57 | C |
| ATOM | 22 | CD2 | LEU | C | 26 | −7.329 | −10.591 | 9.679 | 1.00 | 15.98 | C |
| ATOM | 23 | C | LEU | C | 26 | −5.745 | −9.135 | 6.791 | 1.00 | 15.47 | C |
| ATOM | 24 | O | LEU | C | 26 | −5.796 | −10.352 | 6.573 | 1.00 | 14.79 | O |
| ATOM | 25 | N | VAL | C | 27 | −6.011 | −8.219 | 5.865 | 1.00 | 14.68 | N |
| ATOM | 26 | CA | VAL | C | 27 | −6.391 | −8.594 | 4.504 | 1.00 | 14.46 | C |
| ATOM | 27 | CB | VAL | C | 27 | −6.966 | −7.388 | 3.725 | 1.00 | 14.66 | C |
| ATOM | 28 | CG1 | VAL | C | 27 | −7.305 | −7.781 | 2.293 | 1.00 | 15.29 | C |
| ATOM | 29 | CG2 | VAL | C | 27 | −8.207 | −6.858 | 4.432 | 1.00 | 14.92 | C |
| ATOM | 30 | C | VAL | C | 27 | −5.201 | −9.228 | 3.771 | 1.00 | 14.20 | C |
| ATOM | 31 | O | VAL | C | 27 | −5.366 | −10.212 | 3.051 | 1.00 | 14.01 | O |
| ATOM | 32 | N | VAL | C | 28 | −4.006 | −8.678 | 3.986 | 1.00 | 13.75 | N |
| ATOM | 33 | CA | VAL | C | 28 | −2.783 | −9.258 | 3.421 | 1.00 | 13.27 | C |
| ATOM | 34 | CB | VAL | C | 28 | −1.551 | −8.352 | 3.684 | 1.00 | 13.60 | C |
| ATOM | 35 | CG1 | VAL | C | 28 | −0.245 | −9.059 | 3.293 | 1.00 | 14.53 | C |
| ATOM | 36 | CG2 | VAL | C | 28 | −1.704 | −7.032 | 2.930 | 1.00 | 14.04 | C |
| ATOM | 37 | C | VAL | C | 28 | −2.566 | −10.675 | 3.962 | 1.00 | 12.32 | C |
| ATOM | 38 | O | VAL | C | 28 | −2.357 | −11.614 | 3.191 | 1.00 | 12.09 | O |
| ATOM | 39 | N | ALA | C | 29 | −2.644 | −10.826 | 5.283 | 1.00 | 11.62 | N |
| ATOM | 40 | CA | ALA | C | 29 | −2.504 | −12.137 | 5.916 | 1.00 | 11.05 | C |
| ATOM | 41 | CB | ALA | C | 29 | −2.658 | −12.017 | 7.432 | 1.00 | 11.48 | C |
| ATOM | 42 | C | ALA | C | 29 | −3.505 | −13.151 | 5.347 | 1.00 | 10.65 | C |
| ATOM | 43 | O | ALA | C | 29 | −3.127 | −14.266 | 4.968 | 1.00 | 10.22 | O |
| ATOM | 44 | N | ALA | C | 30 | −4.774 | −12.753 | 5.271 | 1.00 | 10.45 | N |
| ATOM | 45 | CA | ALA | C | 30 | −5.829 | −13.617 | 4.731 | 1.00 | 10.34 | C |
| ATOM | 46 | CB | ALA | C | 30 | −7.184 | −12.954 | 4.880 | 1.00 | 10.82 | C |
| ATOM | 47 | C | ALA | C | 30 | −5.582 | −13.986 | 3.269 | 1.00 | 10.22 | C |
| ATOM | 48 | O | ALA | C | 30 | −5.844 | −15.119 | 2.860 | 1.00 | 10.55 | O |
| ATOM | 49 | N | SER | C | 31 | −5.082 | −13.022 | 2.491 | 1.00 | 9.88 | N |
| ATOM | 50 | CA | SER | C | 31 | −4.752 | −13.250 | 1.081 | 1.00 | 9.45 | C |
| ATOM | 51 | CB | SER | C | 31 | −4.343 | −11.946 | 0.398 | 1.00 | 9.90 | C |
| ATOM | 52 | OG | SER | C | 31 | −5.462 | −11.078 | 0.283 | 1.00 | 10.48 | O |
| ATOM | 53 | C | SER | C | 31 | −3.659 | −14.306 | 0.923 | 1.00 | 9.17 | C |
| ATOM | 54 | O | SER | C | 31 | −3.770 | −15.217 | 0.097 | 1.00 | 8.43 | O |
| ATOM | 55 | N | ILE | C | 32 | −2.609 | −14.184 | 1.728 | 1.00 | 8.62 | N |
| ATOM | 56 | CA | ILE | C | 32 | −1.544 | −15.179 | 1.744 | 1.00 | 8.96 | C |
| ATOM | 57 | CB | ILE | C | 32 | −0.407 | −14.752 | 2.704 | 1.00 | 8.84 | C |
| ATOM | 58 | CG1 | ILE | C | 32 | 0.297 | −13.501 | 2.161 | 1.00 | 8.95 | C |
| ATOM | 59 | CD1 | ILE | C | 32 | 1.168 | −12.812 | 3.185 | 1.00 | 9.39 | C |
| ATOM | 60 | CG2 | ILE | C | 32 | 0.602 | −15.885 | 2.903 | 1.00 | 10.04 | C |
| ATOM | 61 | C | ILE | C | 32 | −2.101 | −16.546 | 2.144 | 1.00 | 9.21 | C |
| ATOM | 62 | O | ILE | C | 32 | −1.808 | −17.559 | 1.506 | 1.00 | 9.19 | O |
| ATOM | 63 | N | ILE | C | 33 | −2.917 | −16.554 | 3.194 | 1.00 | 9.56 | N |
| ATOM | 64 | CA | ILE | C | 33 | −3.494 | −17.792 | 3.715 | 1.00 | 10.37 | C |
| ATOM | 65 | CB | ILE | C | 33 | −4.159 | −17.557 | 5.102 | 1.00 | 10.65 | C |
| ATOM | 66 | CG1 | ILE | C | 33 | −3.065 | −17.554 | 6.175 | 1.00 | 11.79 | C |
| ATOM | 67 | CD1 | ILE | C | 33 | −3.486 | −17.039 | 7.539 | 1.00 | 14.33 | C |
| ATOM | 68 | CG2 | ILE | C | 33 | −5.237 | −18.611 | 5.405 | 1.00 | 11.06 | C |
| ATOM | 69 | C | ILE | C | 33 | −4.412 | −18.479 | 2.696 | 1.00 | 10.53 | C |
| ATOM | 70 | O | ILE | C | 33 | −4.378 | −19.702 | 2.566 | 1.00 | 10.46 | O |
| ATOM | 71 | N | ALA | C | 34 | −5.195 | −17.695 | 1.957 | 1.00 | 10.71 | N |
| ATOM | 72 | CA | ALA | C | 34 | −6.086 | −18.240 | 0.915 | 1.00 | 11.22 | C |
| ATOM | 73 | CB | ALA | C | 34 | −6.957 | −17.137 | 0.324 | 1.00 | 11.45 | C |
| ATOM | 74 | C | ALA | C | 34 | −5.311 | −18.969 | −0.190 | 1.00 | 11.27 | C |
| ATOM | 75 | O | ALA | C | 34 | −5.724 | −20.041 | −0.687 | 1.00 | 12.14 | O |
| ATOM | 76 | N | ILE | C | 35 | −4.165 | −18.410 | −0.555 | 1.00 | 10.66 | N |
| ATOM | 77 | CA | ILE | C | 35 | −3.316 | −19.038 | −1.555 | 1.00 | 10.31 | C |
| ATOM | 78 | CB | ILE | C | 35 | −2.256 | −18.052 | −2.121 | 1.00 | 10.36 | C |
| ATOM | 79 | CG1 | ILE | C | 35 | −2.955 | −16.837 | −2.763 | 1.00 | 10.82 | C |
| ATOM | 80 | CD1 | ILE | C | 35 | −2.036 | −15.639 | −3.048 | 1.00 | 11.05 | C |
| ATOM | 81 | CG2 | ILE | C | 35 | −1.365 | −18.762 | −3.134 | 1.00 | 10.31 | C |
| ATOM | 82 | C | ILE | C | 35 | −2.691 | −20.316 | −0.978 | 1.00 | 9.87 | C |
| ATOM | 83 | O | ILE | C | 35 | −2.714 | −21.367 | −1.625 | 1.00 | 10.38 | O |
| ATOM | 84 | N | LEU | C | 36 | −2.178 | −20.229 | 0.252 | 1.00 | 9.63 | N |
| ATOM | 85 | CA | LEU | C | 36 | −1.580 | −21.373 | 0.925 | 1.00 | 9.11 | C |
| ATOM | 86 | CB | LEU | C | 36 | −1.024 | −20.968 | 2.296 | 1.00 | 8.98 | C |
| ATOM | 87 | CG | LEU | C | 36 | −0.459 | −22.116 | 3.140 | 1.00 | 9.72 | C |
| ATOM | 88 | CD1 | LEU | C | 36 | 0.722 | −22.791 | 2.440 | 1.00 | 10.80 | C |
| ATOM | 89 | CD2 | LEU | C | 36 | −0.071 | −21.650 | 4.545 | 1.00 | 9.71 | C |
| ATOM | 90 | C | LEU | C | 36 | −2.592 | −22.504 | 1.076 | 1.00 | 8.74 | C |
| ATOM | 91 | O | LEU | C | 36 | −2.283 | −23.664 | 0.782 | 1.00 | 8.78 | O |
| ATOM | 92 | N | HIS | C | 37 | −3.800 | −22.155 | 1.514 | 1.00 | 8.52 | N |
| ATOM | 93 | CA | HIS | C | 37 | −4.875 | −23.131 | 1.696 | 1.00 | 8.58 | C |
| ATOM | 94 | CB | HIS | C | 37 | −6.144 | −22.435 | 2.193 | 1.00 | 8.66 | C |
| ATOM | 95 | CG | HIS | C | 37 | −7.177 | −23.367 | 2.744 | 1.00 | 9.62 | C |
| ATOM | 96 | ND1 | HIS | C | 37 | −8.272 | −22.916 | 3.451 | 1.00 | 9.93 | N |
| ATOM | 97 | CE1 | HIS | C | 37 | −9.014 | −23.947 | 3.813 | 1.00 | 10.82 | C |
| ATOM | 98 | NE2 | HIS | C | 37 | −8.440 | −25.050 | 3.372 | 1.00 | 10.52 | N |
| ATOM | 99 | CD2 | HIS | C | 37 | −7.286 | −24.716 | 2.704 | 1.00 | 10.04 | C |
| ATOM | 100 | C | HIS | C | 37 | −5.167 | −23.907 | 0.402 | 1.00 | 8.57 | C |
| ATOM | 101 | O | HIS | C | 37 | −5.305 | −25.133 | 0.435 | 1.00 | 8.87 | O |
| ATOM | 102 | N | LEU | C | 38 | −5.246 | −23.209 | −0.734 | 1.00 | 8.40 | N |
| ATOM | 103 | CA | LEU | C | 38 | −5.524 | −23.905 | −1.994 | 1.00 | 9.18 | C |
| ATOM | 104 | CB | LEU | C | 38 | −5.768 | −22.939 | −3.161 | 1.00 | 8.99 | C |
| ATOM | 105 | CG | LEU | C | 38 | −6.027 | −23.663 | −4.496 | 1.00 | 10.19 | C |
| ATOM | 106 | CD1 | LEU | C | 38 | −7.266 | −24.567 | −4.429 | 1.00 | 12.02 | C |
| ATOM | 107 | CD2 | LEU | C | 38 | −6.145 | −22.693 | −5.655 | 1.00 | 10.88 | C |
| ATOM | 108 | C | LEU | C | 38 | −4.396 | −24.862 | −2.337 | 1.00 | 8.77 | C |
| ATOM | 109 | O | LEU | C | 38 | −4.640 | −26.003 | −2.726 | 1.00 | 8.58 | O |
| ATOM | 110 | N | ILE | C | 39 | −3.158 | −24.399 | −2.175 | 1.00 | 8.74 | N |
| ATOM | 111 | CA | ILE | C | 39 | −1.999 | −25.237 | −2.462 | 1.00 | 9.07 | C |
| ATOM | 112 | CB | ILE | C | 39 | −0.681 | −24.467 | −2.243 | 1.00 | 9.14 | C |
| ATOM | 113 | CG1 | ILE | C | 39 | −0.562 | −23.339 | −3.274 | 1.00 | 9.55 | C |
| ATOM | 114 | CD1 | ILE | C | 39 | 0.643 | −22.433 | −3.069 | 1.00 | 11.01 | C |
| ATOM | 115 | CG2 | ILE | C | 39 | 0.521 | −25.416 | −2.322 | 1.00 | 10.12 | C |
| ATOM | 116 | C | ILE | C | 39 | −2.029 | −26.498 | −1.601 | 1.00 | 9.01 | C |
| ATOM | 117 | O | ILE | C | 39 | −1.897 | −27.614 | −2.112 | 1.00 | 9.04 | O |
| ATOM | 118 | N | LEU | C | 40 | −2.234 | −26.317 | −0.296 | 1.00 | 9.18 | N |
| ATOM | 119 | CA | LEU | C | 40 | −2.273 | −27.440 | 0.636 | 1.00 | 9.35 | C |
| ATOM | 120 | CB | LEU | C | 40 | −2.451 | −26.938 | 2.069 | 1.00 | 9.12 | C |
| ATOM | 121 | CG | LEU | C | 40 | −1.261 | −26.211 | 2.693 | 1.00 | 9.08 | C |
| ATOM | 122 | CD1 | LEU | C | 40 | −1.649 | −25.664 | 4.065 | 1.00 | 9.37 | C |
| ATOM | 123 | CD2 | LEU | C | 40 | −0.027 | −27.108 | 2.797 | 1.00 | 10.59 | C |
| ATOM | 124 | C | LEU | C | 40 | −3.386 | −28.426 | 0.299 | 1.00 | 9.78 | C |
| ATOM | 125 | O | LEU | C | 40 | −3.173 | −29.642 | 0.324 | 1.00 | 9.64 | O |
| ATOM | 126 | N | TRP | C | 41 | −4.565 | −27.894 | −0.017 | 1.00 | 10.32 | N |
| ATOM | 127 | CA | TRP | C | 41 | −5.722 | −28.729 | −0.332 | 1.00 | 11.12 | C |
| ATOM | 128 | CB | TRP | C | 41 | −7.001 | −27.888 | −0.420 | 1.00 | 11.48 | C |
| ATOM | 129 | CG | TRP | C | 41 | −8.229 | −28.703 | −0.697 | 1.00 | 11.67 | C |
| ATOM | 130 | CD1 | TRP | C | 41 | −8.974 | −29.409 | 0.210 | 1.00 | 12.59 | C |
| ATOM | 131 | NE1 | TRP | C | 41 | −10.031 | −30.028 | −0.426 | 1.00 | 12.79 | N |
| ATOM | 132 | CE2 | TRP | C | 41 | −9.979 | −29.731 | −1.762 | 1.00 | 11.96 | C |
| ATOM | 133 | CD2 | TRP | C | 41 | −8.857 | −28.896 | −1.970 | 1.00 | 11.32 | C |
| ATOM | 134 | CE3 | TRP | C | 41 | −8.577 | −28.450 | −3.267 | 1.00 | 13.18 | C |
| ATOM | 135 | CZ3 | TRP | C | 41 | −9.416 | −28.844 | −4.306 | 1.00 | 12.40 | C |
| ATOM | 136 | CH2 | TRP | C | 41 | −10.526 | −29.672 | −4.068 | 1.00 | 12.49 | C |
| ATOM | 137 | CZ2 | TRP | C | 41 | −10.823 | −30.127 | −2.808 | 1.00 | 11.32 | C |
| ATOM | 138 | C | TRP | C | 41 | −5.514 | −29.543 | −1.614 | 1.00 | 11.78 | C |
| ATOM | 139 | O | TRP | C | 41 | −5.838 | −30.733 | −1.653 | 1.00 | 11.32 | O |
| ATOM | 140 | N | ILE | C | 42 | −4.966 | −28.907 | −2.648 | 1.00 | 12.72 | N |
| ATOM | 141 | CA | ILE | C | 42 | −4.666 | −29.600 | −3.905 | 1.00 | 14.19 | C |
| ATOM | 142 | CB | ILE | C | 42 | −4.106 | −28.633 | −4.978 | 1.00 | 14.02 | C |
| ATOM | 143 | CG1 | ILE | C | 42 | −5.206 | −27.683 | −5.462 | 1.00 | 14.99 | C |
| ATOM | 144 | CD1 | ILE | C | 42 | −4.734 | −26.637 | −6.458 | 1.00 | 14.99 | C |
| ATOM | 145 | CG2 | ILE | C | 42 | −3.501 | −29.412 | −6.154 | 1.00 | 14.79 | C |
| ATOM | 146 | C | ILE | C | 42 | −3.686 | −30.750 | −3.662 | 1.00 | 14.74 | C |
| ATOM | 147 | O | ILE | C | 42 | −3.905 | −31.867 | −4.138 | 1.00 | 15.02 | O |
| ATOM | 148 | N | LEU | C | 43 | −2.621 | −30.474 | −2.910 | 1.00 | 15.42 | N |
| ATOM | 149 | CA | LEU | C | 43 | −1.626 | −31.495 | −2.569 | 1.00 | 16.34 | C |
| ATOM | 150 | CB | LEU | C | 43 | −0.416 | −30.864 | −1.874 | 1.00 | 16.38 | C |
| ATOM | 151 | CG | LEU | C | 43 | 0.441 | −29.948 | −2.756 | 1.00 | 16.75 | C |
| ATOM | 152 | CD1 | LEU | C | 43 | 1.430 | −29.153 | −1.913 | 1.00 | 16.91 | C |
| ATOM | 153 | CD2 | LEU | C | 43 | 1.175 | −30.737 | −3.836 | 1.00 | 17.75 | C |
| ATOM | 154 | C | LEU | C | 43 | −2.209 | −32.633 | −1.728 | 1.00 | 17.21 | C |
| ATOM | 155 | O | LEU | C | 43 | −1.832 | −33.791 | −1.910 | 1.00 | 17.53 | O |
| ATOM | 156 | N | ASP | C | 44 | −3.121 | −32.299 | −0.816 | 1.00 | 17.98 | N |
| ATOM | 157 | CA | ASP | C | 44 | −3.817 | −33.305 | −0.016 | 1.00 | 19.24 | C |
| ATOM | 158 | CB | ASP | C | 44 | −4.702 | −32.649 | 1.048 | 1.00 | 19.49 | C |
| ATOM | 159 | CG | ASP | C | 44 | −5.397 | −33.668 | 1.940 | 1.00 | 20.20 | C |
| ATOM | 160 | OD1 | ASP | C | 44 | −6.641 | −33.733 | 1.914 | 1.00 | 22.10 | O |
| ATOM | 161 | OD2 | ASP | C | 44 | −4.696 | −34.411 | 2.652 | 1.00 | 22.01 | O |
| ATOM | 162 | C | ASP | C | 44 | −4.664 | −34.216 | −0.903 | 1.00 | 19.85 | C |
| ATOM | 163 | O | ASP | C | 44 | −4.666 | −35.435 | −0.722 | 1.00 | 19.96 | O |
| ATOM | 164 | N | ARG | C | 45 | −5.362 | −33.614 | −1.866 | 1.00 | 20.86 | N |
| ATOM | 165 | CA | ARG | C | 45 | −6.260 | −34.343 | −2.767 | 1.00 | 22.11 | C |
| ATOM | 166 | CB | ARG | C | 45 | −7.168 | −33.376 | −3.534 | 1.00 | 22.28 | C |
| ATOM | 167 | CG | ARG | C | 45 | −8.191 | −32.631 | −2.685 | 1.00 | 23.79 | C |
| ATOM | 168 | CD | ARG | C | 45 | −9.325 | −33.534 | −2.216 | 1.00 | 27.01 | C |
| ATOM | 169 | NE | ARG | C | 45 | −9.042 | −34.152 | −0.924 | 1.00 | 28.83 | N |
| ATOM | 170 | CZ | ARG | C | 45 | −9.628 | −35.259 | −0.475 | 1.00 | 30.46 | C |
| ATOM | 171 | NH1 | ARG | C | 45 | −10.530 | −35.891 | −1.218 | 1.00 | 31.13 | N |
| ATOM | 172 | NH2 | ARG | C | 45 | −9.300 | −35.744 | 0.717 | 1.00 | 30.77 | N |
| ATOM | 173 | C | ARG | C | 45 | −5.508 | −35.234 | −3.750 | 1.00 | 22.85 | C |
| ATOM | 174 | O | ARG | C | 45 | −5.949 | −36.348 | −4.044 | 1.00 | 22.94 | O |
| ATOM | 175 | N | LEU | C | 46 | −4.384 | −34.734 | −4.261 | 1.00 | 23.71 | N |
| ATOM | 176 | CA | LEU | C | 46 | −3.526 | −35.506 | −5.160 | 1.00 | 24.51 | C |
| ATOM | 177 | CB | LEU | C | 46 | −2.489 | −34.604 | −5.840 | 1.00 | 24.74 | C |
| ATOM | 178 | CG | LEU | C | 46 | −2.955 | −33.455 | −6.744 | 1.00 | 25.18 | C |
| ATOM | 179 | CD1 | LEU | C | 46 | −1.768 | −32.588 | −7.137 | 1.00 | 25.61 | C |
| ATOM | 180 | CD2 | LEU | C | 46 | −3.698 | −33.950 | −7.981 | 1.00 | 25.87 | C |
| ATOM | 181 | C | LEU | C | 46 | −2.824 | −36.645 | −4.423 | 1.00 | 24.99 | C |
| ATOM | 182 | O | LEU | C | 46 | −2.430 | −36.507 | −3.263 | 1.00 | 25.33 | O |
| ATOM | 183 | N | NH2 | C | 47 | −2.253 | −37.679 | −4.862 | 1.00 | 25.50 | N |
| ATOM | 184 | BR4 | BRB | D | 1 | −14.373 | 0.276 | 1.864 | 1.00 | 35.58 | BR |
| ATOM | 185 | C4 | BRB | D | 1 | −14.792 | −1.078 | 3.102 | 1.00 | 30.63 | C |
| ATOM | 186 | C3 | BRB | D | 1 | −16.095 | −1.563 | 3.171 | 1.00 | 31.13 | C |
| ATOM | 187 | C2 | BRB | D | 1 | −16.400 | −2.564 | 4.086 | 1.00 | 31.07 | C |
| ATOM | 188 | C5 | BRB | D | 1 | −13.790 | −1.566 | 3.934 | 1.00 | 31.30 | C |
| ATOM | 189 | C6 | BRB | D | 1 | −14.099 | −2.564 | 4.851 | 1.00 | 31.07 | C |
| ATOM | 190 | C1 | BRB | D | 1 | −15.399 | −3.056 | 4.924 | 1.00 | 29.91 | C |
| ATOM | 191 | C7 | BRB | D | 1 | −15.719 | −4.136 | 5.926 | 1.00 | 25.19 | C |
| ATOM | 192 | O1 | BRB | D | 1 | −14.739 | −5.168 | 5.776 | 1.00 | 23.79 | O |
| ATOM | 193 | N | PRO | D | 25 | −16.733 | −4.188 | 6.683 | 1.00 | 19.96 | N |
| ATOM | 194 | CA | PRO | D | 25 | −16.996 | −5.300 | 7.595 | 1.00 | 18.27 | C |
| ATOM | 195 | CB | PRO | D | 25 | −18.368 | −4.950 | 8.161 | 1.00 | 18.64 | C |
| ATOM | 196 | CG | PRO | D | 25 | −18.350 | −3.446 | 8.227 | 1.00 | 19.09 | C |
| ATOM | 197 | CD | PRO | D | 25 | −17.444 | −2.964 | 7.107 | 1.00 | 19.39 | C |
| ATOM | 198 | C | PRO | D | 25 | −17.026 | −6.667 | 6.903 | 1.00 | 17.45 | C |
| ATOM | 199 | O | PRO | D | 25 | −16.548 | −7.650 | 7.476 | 1.00 | 16.71 | O |
| ATOM | 200 | N | LEU | D | 26 | −17.566 | −6.721 | 5.683 | 1.00 | 16.21 | N |
| ATOM | 201 | CA | LEU | D | 26 | −17.613 | −7.963 | 4.910 | 1.00 | 15.13 | C |
| ATOM | 202 | CB | LEU | D | 26 | −18.346 | −7.753 | 3.578 | 1.00 | 15.06 | C |
| ATOM | 203 | CG | LEU | D | 26 | −18.386 | −8.946 | 2.609 | 1.00 | 15.42 | C |
| ATOM | 204 | CD1 | LEU | D | 26 | −19.061 | −10.165 | 3.242 | 1.00 | 15.08 | C |
| ATOM | 205 | CD2 | LEU | D | 26 | −19.066 | −8.558 | 1.301 | 1.00 | 14.92 | C |
| ATOM | 206 | C | LEU | D | 26 | −16.221 | −8.545 | 4.650 | 1.00 | 14.40 | C |
| ATOM | 207 | O | LEU | D | 26 | −16.006 | −9.747 | 4.811 | 1.00 | 13.62 | O |
| ATOM | 208 | N | VAL | D | 27 | −15.289 | −7.690 | 4.239 | 1.00 | 14.01 | N |
| ATOM | 209 | CA | VAL | D | 27 | −13.935 | −8.127 | 3.905 | 1.00 | 13.83 | C |
| ATOM | 210 | CB | VAL | D | 27 | −13.172 | −7.059 | 3.082 | 1.00 | 13.96 | C |
| ATOM | 211 | CG1 | VAL | D | 27 | −11.798 | −7.571 | 2.658 | 1.00 | 14.39 | C |
| ATOM | 212 | CG2 | VAL | D | 27 | −13.974 | −6.686 | 1.854 | 1.00 | 14.54 | C |
| ATOM | 213 | C | VAL | D | 27 | −13.167 | −8.513 | 5.168 | 1.00 | 13.53 | C |
| ATOM | 214 | O | VAL | D | 27 | −12.424 | −9.494 | 5.167 | 1.00 | 13.34 | O |
| ATOM | 215 | N | VAL | D | 28 | −13.363 | −7.747 | 6.240 | 1.00 | 12.94 | N |
| ATOM | 216 | CA | VAL | D | 28 | −12.787 | −8.081 | 7.546 | 1.00 | 12.94 | C |
| ATOM | 217 | CB | VAL | D | 28 | −13.096 | −6.990 | 8.604 | 1.00 | 13.24 | C |
| ATOM | 218 | CG1 | VAL | D | 28 | −12.632 | −7.423 | 9.992 | 1.00 | 12.94 | C |
| ATOM | 219 | CG2 | VAL | D | 28 | −12.437 | −5.680 | 8.216 | 1.00 | 13.55 | C |
| ATOM | 220 | C | VAL | D | 28 | −13.286 | −9.451 | 8.025 | 1.00 | 12.34 | C |
| ATOM | 221 | O | VAL | D | 28 | −12.483 | −10.318 | 8.389 | 1.00 | 12.38 | O |
| ATOM | 222 | N | ALA | D | 29 | −14.607 | −9.637 | 8.021 | 1.00 | 11.69 | N |
| ATOM | 223 | CA | ALA | D | 29 | −15.207 | −10.917 | 8.400 | 1.00 | 10.98 | C |
| ATOM | 224 | CB | ALA | D | 29 | −16.727 | −10.840 | 8.322 | 1.00 | 11.45 | C |
| ATOM | 225 | C | ALA | D | 29 | −14.678 | −12.078 | 7.554 | 1.00 | 10.83 | C |
| ATOM | 226 | O | ALA | D | 29 | −14.301 | −13.122 | 8.097 | 1.00 | 10.38 | O |
| ATOM | 227 | N | ALA | D | 30 | −14.630 | −11.888 | 6.235 | 1.00 | 10.33 | N |
| ATOM | 228 | CA | ALA | D | 30 | −14.126 | −12.931 | 5.330 | 1.00 | 10.26 | C |
| ATOM | 229 | CB | ALA | D | 30 | −14.329 | −12.527 | 3.880 | 1.00 | 10.54 | C |
| ATOM | 230 | C | ALA | D | 30 | −12.662 | −13.278 | 5.583 | 1.00 | 10.00 | C |
| ATOM | 231 | O | ALA | D | 30 | −12.272 | −14.445 | 5.512 | 1.00 | 9.93 | O |
| ATOM | 232 | N | SER | D | 31 | −11.863 | −12.257 | 5.875 | 1.00 | 9.86 | N |
| ATOM | 233 | CA | SER | D | 31 | −10.448 | −12.436 | 6.181 | 1.00 | 9.97 | C |
| ATOM | 234 | CB | SER | D | 31 | −9.771 | −11.077 | 6.365 | 1.00 | 10.15 | C |
| ATOM | 235 | OG | SER | D | 31 | −9.662 | −10.413 | 5.122 | 1.00 | 11.01 | O |
| ATOM | 236 | C | SER | D | 31 | −10.261 | −13.309 | 7.419 | 1.00 | 9.68 | C |
| ATOM | 237 | O | SER | D | 31 | −9.468 | −14.258 | 7.407 | 1.00 | 9.49 | O |
| ATOM | 238 | N | ILE | D | 32 | −11.013 | −12.991 | 8.475 | 1.00 | 9.57 | N |
| ATOM | 239 | CA | ILE | D | 32 | −11.002 | −13.765 | 9.716 | 1.00 | 10.01 | C |
| ATOM | 240 | CB | ILE | D | 32 | −11.902 | −13.092 | 10.789 | 1.00 | 10.12 | C |
| ATOM | 241 | CG1 | ILE | D | 32 | −11.302 | −11.741 | 11.192 | 1.00 | 10.77 | C |
| ATOM | 242 | CD1 | ILE | D | 32 | −12.291 | −10.791 | 11.879 | 1.00 | 13.07 | C |
| ATOM | 243 | CG2 | ILE | D | 32 | −12.096 | −14.001 | 12.012 | 1.00 | 10.38 | C |
| ATOM | 244 | C | ILE | D | 32 | −11.457 | −15.198 | 9.440 | 1.00 | 9.99 | C |
| ATOM | 245 | O | ILE | D | 32 | −10.825 | −16.159 | 9.881 | 1.00 | 9.84 | O |
| ATOM | 246 | N | ILE | D | 33 | −12.538 | −15.331 | 8.676 | 1.00 | 10.07 | N |
| ATOM | 247 | CA | ILE | D | 33 | −13.106 | −16.645 | 8.370 | 1.00 | 10.61 | C |
| ATOM | 248 | CB | ILE | D | 33 | −14.515 | −16.507 | 7.740 | 1.00 | 11.03 | C |
| ATOM | 249 | CG1 | ILE | D | 33 | −15.517 | −16.171 | 8.851 | 1.00 | 11.77 | C |
| ATOM | 250 | CD1 | ILE | D | 33 | −16.836 | −15.593 | 8.387 | 1.00 | 13.82 | C |
| ATOM | 251 | CG2 | ILE | D | 33 | −14.910 | −17.776 | 6.993 | 1.00 | 11.25 | C |
| ATOM | 252 | C | ILE | D | 33 | −12.144 | −17.499 | 7.534 | 1.00 | 10.56 | C |
| ATOM | 253 | O | ILE | D | 33 | −12.027 | −18.707 | 7.762 | 1.00 | 10.01 | O |
| ATOM | 254 | N | ALA | D | 34 | −11.437 | −16.866 | 6.600 | 1.00 | 10.68 | N |
| ATOM | 255 | CA | ALA | D | 34 | −10.418 | −17.554 | 5.800 | 1.00 | 10.97 | C |
| ATOM | 256 | CB | ALA | D | 34 | −9.833 | −16.614 | 4.745 | 1.00 | 11.33 | C |
| ATOM | 257 | C | ALA | D | 34 | −9.305 | −18.145 | 6.678 | 1.00 | 10.79 | C |
| ATOM | 258 | O | ALA | D | 34 | −8.894 | −19.291 | 6.485 | 1.00 | 11.04 | O |
| ATOM | 259 | N | ILE | D | 35 | −8.831 | −17.360 | 7.645 | 1.00 | 10.34 | N |
| ATOM | 260 | CA | ILE | D | 35 | −7.825 | −17.815 | 8.611 | 1.00 | 10.14 | C |
| ATOM | 261 | CB | ILE | D | 35 | −7.321 | −16.637 | 9.502 | 1.00 | 10.53 | C |
| ATOM | 262 | CG1 | ILE | D | 35 | −6.674 | −15.557 | 8.613 | 1.00 | 11.81 | C |
| ATOM | 263 | CD1 | ILE | D | 35 | −6.563 | −14.180 | 9.246 | 1.00 | 14.05 | C |
| ATOM | 264 | CG2 | ILE | D | 35 | −6.343 | −17.137 | 10.580 | 1.00 | 10.49 | C |
| ATOM | 265 | C | ILE | D | 35 | −8.378 | −18.972 | 9.453 | 1.00 | 9.31 | C |
| ATOM | 266 | O | ILE | D | 35 | −7.725 | −20.011 | 9.607 | 1.00 | 9.36 | O |
| ATOM | 267 | N | LEU | D | 36 | −9.589 | −18.795 | 9.974 | 1.00 | 8.62 | N |
| ATOM | 268 | CA | LEU | D | 36 | −10.262 | −19.846 | 10.728 | 1.00 | 8.20 | C |
| ATOM | 269 | CB | LEU | D | 36 | −11.646 | −19.379 | 11.182 | 1.00 | 8.04 | C |
| ATOM | 270 | CG | LEU | D | 36 | −12.454 | −20.411 | 11.975 | 1.00 | 7.81 | C |
| ATOM | 271 | CD1 | LEU | D | 36 | −11.785 | −20.722 | 13.309 | 1.00 | 9.12 | C |
| ATOM | 272 | CD2 | LEU | D | 36 | −13.874 | −19.907 | 12.183 | 1.00 | 8.20 | C |
| ATOM | 273 | C | LEU | D | 36 | −10.392 | −21.126 | 9.905 | 1.00 | 8.07 | C |
| ATOM | 274 | O | LEU | D | 36 | −10.088 | −22.212 | 10.388 | 1.00 | 8.03 | O |
| ATOM | 275 | N | HIS | D | 37 | −10.834 | −20.982 | 8.659 | 1.00 | 7.93 | N |
| ATOM | 276 | CA | HIS | D | 37 | −11.023 | −22.121 | 7.766 | 1.00 | 7.74 | C |
| ATOM | 277 | CB | HIS | D | 37 | −11.540 | −21.644 | 6.407 | 1.00 | 7.96 | C |
| ATOM | 278 | CG | HIS | D | 37 | −12.063 | −22.745 | 5.537 | 1.00 | 9.06 | C |
| ATOM | 279 | ND1 | HIS | D | 37 | −12.683 | −22.498 | 4.331 | 1.00 | 10.90 | N |
| ATOM | 280 | CE1 | HIS | D | 37 | −13.037 | −23.646 | 3.779 | 1.00 | 11.47 | C |
| ATOM | 281 | NE2 | HIS | D | 37 | −12.682 | −24.627 | 4.589 | 1.00 | 10.30 | N |
| ATOM | 282 | CD2 | HIS | D | 37 | −12.076 | −24.090 | 5.700 | 1.00 | 9.55 | C |
| ATOM | 283 | C | HIS | D | 37 | −9.731 | −22.929 | 7.599 | 1.00 | 7.93 | C |
| ATOM | 284 | O | HIS | D | 37 | −9.744 | −24.157 | 7.725 | 1.00 | 7.69 | O |
| ATOM | 285 | N | LEU | D | 38 | −8.613 | −22.246 | 7.347 | 1.00 | 7.89 | N |
| ATOM | 286 | CA | LEU | D | 38 | −7.343 | −22.954 | 7.199 | 1.00 | 8.27 | C |
| ATOM | 287 | CB | LEU | D | 38 | −6.205 | −22.002 | 6.819 | 1.00 | 8.69 | C |
| ATOM | 288 | CG | LEU | D | 38 | −4.811 | −22.638 | 6.843 | 1.00 | 8.73 | C |
| ATOM | 289 | CD1 | LEU | D | 38 | −4.645 | −23.686 | 5.731 | 1.00 | 9.89 | C |
| ATOM | 290 | CD2 | LEU | D | 38 | −3.715 | −21.580 | 6.778 | 1.00 | 9.16 | C |
| ATOM | 291 | C | LEU | D | 38 | −7.005 | −23.746 | 8.466 | 1.00 | 8.28 | C |
| ATOM | 292 | O | LEU | D | 38 | −6.679 | −24.928 | 8.392 | 1.00 | 8.04 | O |
| ATOM | 293 | N | ILE | D | 39 | −7.122 | −23.096 | 9.621 | 1.00 | 7.91 | N |
| ATOM | 294 | CA | ILE | D | 39 | −6.823 | −23.732 | 10.906 | 1.00 | 8.14 | C |
| ATOM | 295 | CB | ILE | D | 39 | −6.989 | −22.733 | 12.068 | 1.00 | 8.40 | C |
| ATOM | 296 | CG1 | ILE | D | 39 | −5.923 | −21.635 | 11.959 | 1.00 | 8.14 | C |
| ATOM | 297 | CD1 | ILE | D | 39 | −6.212 | −20.379 | 12.773 | 1.00 | 9.48 | C |
| ATOM | 298 | CG2 | ILE | D | 39 | −6.921 | −23.456 | 13.417 | 1.00 | 8.49 | C |
| ATOM | 299 | C | ILE | D | 39 | −7.694 | −24.977 | 11.122 | 1.00 | 7.92 | C |
| ATOM | 300 | O | ILE | D | 39 | −7.183 | −26.060 | 11.435 | 1.00 | 8.19 | O |
| ATOM | 301 | N | LEU | D | 40 | −9.000 | −24.831 | 10.910 | 1.00 | 7.89 | N |
| ATOM | 302 | CA | LEU | D | 40 | −9.933 | −25.938 | 11.085 | 1.00 | 7.70 | C |
| ATOM | 303 | CB | LEU | D | 40 | −11.377 | −25.468 | 10.916 | 1.00 | 7.93 | C |
| ATOM | 304 | CG | LEU | D | 40 | −11.891 | −24.464 | 11.944 | 1.00 | 7.72 | C |
| ATOM | 305 | CD1 | LEU | D | 40 | −13.343 | −24.130 | 11.625 | 1.00 | 8.62 | C |
| ATOM | 306 | CD2 | LEU | D | 40 | −11.739 | −24.984 | 13.380 | 1.00 | 9.15 | C |
| ATOM | 307 | C | LEU | D | 40 | −9.649 | −27.081 | 10.124 | 1.00 | 7.87 | C |
| ATOM | 308 | O | LEU | D | 40 | −9.725 | −28.248 | 10.500 | 1.00 | 7.71 | O |
| ATOM | 309 | N | TRP | D | 41 | −9.311 | −26.742 | 8.882 | 1.00 | 8.10 | N |
| ATOM | 310 | CA | TRP | D | 41 | −9.051 | −27.769 | 7.882 | 1.00 | 8.74 | C |
| ATOM | 311 | CB | TRP | D | 41 | −8.973 | −27.170 | 6.476 | 1.00 | 9.25 | C |
| ATOM | 312 | CG | TRP | D | 41 | −8.653 | −28.179 | 5.433 | 1.00 | 9.70 | C |
| ATOM | 313 | CD1 | TRP | D | 41 | −9.515 | −29.071 | 4.850 | 1.00 | 10.72 | C |
| ATOM | 314 | NE1 | TRP | D | 41 | −8.842 | −29.849 | 3.937 | 1.00 | 10.48 | N |
| ATOM | 315 | CE2 | TRP | D | 41 | −7.525 | −29.464 | 3.918 | 1.00 | 9.97 | C |
| ATOM | 316 | CD2 | TRP | D | 41 | −7.374 | −28.414 | 4.849 | 1.00 | 9.70 | C |
| ATOM | 317 | CE3 | TRP | D | 41 | −6.111 | −27.837 | 5.020 | 1.00 | 10.75 | C |
| ATOM | 318 | CZ3 | TRP | D | 41 | −5.052 | −28.318 | 4.269 | 1.00 | 9.67 | C |
| ATOM | 319 | CH2 | TRP | D | 41 | −5.231 | −29.361 | 3.350 | 1.00 | 10.26 | C |
| ATOM | 320 | CZ2 | TRP | D | 41 | −6.455 | −29.947 | 3.158 | 1.00 | 10.06 | C |
| ATOM | 321 | C | TRP | D | 41 | −7.798 | −28.566 | 8.239 | 1.00 | 8.75 | C |
| ATOM | 322 | O | TRP | D | 41 | −7.794 | −29.789 | 8.148 | 1.00 | 9.04 | O |
| ATOM | 323 | N | ILE | D | 42 | −6.748 | −27.870 | 8.666 | 1.00 | 8.96 | N |
| ATOM | 324 | CA | ILE | D | 42 | −5.539 | −28.539 | 9.154 | 1.00 | 9.29 | C |
| ATOM | 325 | CB | ILE | D | 42 | −4.459 | −27.518 | 9.592 | 1.00 | 9.28 | C |
| ATOM | 326 | CG1 | ILE | D | 42 | −3.913 | −26.779 | 8.362 | 1.00 | 9.46 | C |
| ATOM | 327 | CD1 | ILE | D | 42 | −3.075 | −25.562 | 8.673 | 1.00 | 11.65 | C |
| ATOM | 328 | CG2 | ILE | D | 42 | −3.330 | −28.210 | 10.375 | 1.00 | 10.00 | C |
| ATOM | 329 | C | ILE | D | 42 | −5.872 | −29.520 | 10.289 | 1.00 | 9.21 | C |
| ATOM | 330 | O | ILE | D | 42 | −5.453 | −30.679 | 10.258 | 1.00 | 9.00 | O |
| ATOM | 331 | N | LEU | D | 43 | −6.639 | −29.058 | 11.274 | 1.00 | 9.49 | N |
| ATOM | 332 | CA | LEU | D | 43 | −7.005 | −29.900 | 12.419 | 1.00 | 10.37 | C |
| ATOM | 333 | CB | LEU | D | 43 | −7.752 | −29.082 | 13.474 | 1.00 | 10.18 | C |
| ATOM | 334 | CG | LEU | D | 43 | −6.930 | −27.971 | 14.132 | 1.00 | 9.46 | C |
| ATOM | 335 | CD1 | LEU | D | 43 | −7.836 | −27.023 | 14.919 | 1.00 | 10.40 | C |
| ATOM | 336 | CD2 | LEU | D | 43 | −5.798 | −28.530 | 15.015 | 1.00 | 9.52 | C |
| ATOM | 337 | C | LEU | D | 43 | −7.823 | −31.116 | 11.996 | 1.00 | 11.25 | C |
| ATOM | 338 | O | LEU | D | 43 | −7.636 | −32.214 | 12.527 | 1.00 | 11.31 | O |
| ATOM | 339 | N | ASP | D | 44 | −8.719 | −30.910 | 11.037 | 1.00 | 11.95 | N |
| ATOM | 340 | CA | ASP | D | 44 | −9.490 | −31.995 | 10.432 | 1.00 | 13.12 | C |
| ATOM | 341 | CB | ASP | D | 44 | −10.477 | −31.430 | 9.402 | 1.00 | 13.59 | C |
| ATOM | 342 | CG | ASP | D | 44 | −11.237 | −32.521 | 8.666 | 1.00 | 15.15 | C |
| ATOM | 343 | OD1 | ASP | D | 44 | −12.117 | −33.151 | 9.286 | 1.00 | 18.20 | O |
| ATOM | 344 | OD2 | ASP | D | 44 | −10.947 | −32.750 | 7.472 | 1.00 | 18.47 | O |
| ATOM | 345 | C | ASP | D | 44 | −8.578 | −33.034 | 9.765 | 1.00 | 13.75 | C |
| ATOM | 346 | O | ASP | D | 44 | −8.789 | −34.242 | 9.924 | 1.00 | 13.58 | O |
| ATOM | 347 | N | ARG | D | 45 | −7.567 | −32.566 | 9.032 | 1.00 | 14.28 | N |
| ATOM | 348 | CA | ARG | D | 45 | −6.653 | −33.465 | 8.323 | 1.00 | 15.01 | C |
| ATOM | 349 | CB | ARG | D | 45 | −5.834 | −32.725 | 7.256 | 1.00 | 15.36 | C |
| ATOM | 350 | CG | ARG | D | 45 | −6.658 | −32.035 | 6.154 | 1.00 | 17.20 | C |
| ATOM | 351 | CD | ARG | D | 45 | −7.981 | −32.745 | 5.825 | 1.00 | 21.45 | C |
| ATOM | 352 | NE | ARG | D | 45 | −7.799 | −33.993 | 5.093 | 1.00 | 25.07 | N |
| ATOM | 353 | CZ | ARG | D | 45 | −8.683 | −34.988 | 5.066 | 1.00 | 26.55 | C |
| ATOM | 354 | NH1 | ARG | D | 45 | −9.820 | −34.904 | 5.748 | 1.00 | 26.88 | N |
| ATOM | 355 | NH2 | ARG | D | 45 | −8.420 | −36.081 | 4.364 | 1.00 | 28.24 | N |
| ATOM | 356 | C | ARG | D | 45 | −5.740 | −34.232 | 9.276 | 1.00 | 15.23 | C |
| ATOM | 357 | O | ARG | D | 45 | −5.384 | −35.378 | 9.009 | 1.00 | 15.71 | O |
| ATOM | 358 | N | LEU | D | 46 | −5.377 | −33.602 | 10.391 | 1.00 | 15.21 | N |
| ATOM | 359 | CA | LEU | D | 46 | −4.585 | −34.262 | 11.424 | 1.00 | 15.70 | C |
| ATOM | 360 | CB | LEU | D | 46 | −4.075 | −33.245 | 12.447 | 1.00 | 15.57 | C |
| ATOM | 361 | CG | LEU | D | 46 | −3.034 | −32.215 | 12.001 | 1.00 | 16.15 | C |
| ATOM | 362 | CD1 | LEU | D | 46 | −2.751 | −31.251 | 13.145 | 1.00 | 17.23 | C |
| ATOM | 363 | CD2 | LEU | D | 46 | −1.744 | −32.878 | 11.534 | 1.00 | 17.69 | C |
| ATOM | 364 | C | LEU | D | 46 | −5.400 | −35.338 | 12.134 | 1.00 | 15.57 | C |
| ATOM | 365 | O | LEU | D | 46 | −4.842 | −36.291 | 12.677 | 1.00 | 16.59 | O |
| ATOM | 366 | N | NH2 | D | 47 | −6.659 | −35.297 | 12.187 | 1.00 | 15.14 | N |
| ATOM | 367 | BR4 | BRB | E | 1 | −11.467 | −0.560 | −5.880 | 1.00 | 28.26 | BR |
| ATOM | 368 | C4 | BRB | E | 1 | −12.669 | −2.013 | −5.988 | 1.00 | 22.76 | C |
| ATOM | 369 | C3 | BRB | E | 1 | −13.373 | −2.421 | −4.858 | 1.00 | 23.50 | C |
| ATOM | 370 | C2 | BRB | E | 1 | −14.261 | −3.492 | −4.945 | 1.00 | 23.11 | C |
| ATOM | 371 | C5 | BRB | E | 1 | −12.835 | −2.658 | −7.208 | 1.00 | 22.98 | C |
| ATOM | 372 | C6 | BRB | E | 1 | −13.718 | −3.728 | −7.294 | 1.00 | 22.70 | C |
| ATOM | 373 | C1 | BRB | E | 1 | −14.429 | −4.143 | −6.166 | 1.00 | 21.79 | C |
| ATOM | 374 | C7 | BRB | E | 1 | −15.393 | −5.299 | −6.277 | 1.00 | 17.77 | C |
| ATOM | 375 | O1 | BRB | E | 1 | −15.249 | −6.134 | −5.124 | 1.00 | 14.30 | O |
| ATOM | 376 | N | PRO | E | 25 | −16.169 | −5.574 | −7.240 | 1.00 | 13.90 | N |
| ATOM | 377 | CA | PRO | E | 25 | −17.056 | −6.731 | −7.155 | 1.00 | 12.63 | C |
| ATOM | 378 | CB | PRO | E | 25 | −17.807 | −6.680 | −8.485 | 1.00 | 12.78 | C |
| ATOM | 379 | CG | PRO | E | 25 | −17.960 | −5.196 | −8.717 | 1.00 | 13.62 | C |
| ATOM | 380 | CD | PRO | E | 25 | −16.677 | −4.576 | −8.204 | 1.00 | 13.42 | C |
| ATOM | 381 | C | PRO | E | 25 | −16.328 | −8.065 | −6.964 | 1.00 | 12.19 | C |
| ATOM | 382 | O | PRO | E | 25 | −16.849 | −8.944 | −6.275 | 1.00 | 12.10 | O |
| ATOM | 383 | N | LEU | E | 26 | −15.135 | −8.202 | −7.543 | 1.00 | 11.26 | N |
| ATOM | 384 | CA | LEU | E | 26 | −14.328 | −9.416 | −7.365 | 1.00 | 10.80 | C |
| ATOM | 385 | CB | LEU | E | 26 | −13.010 | −9.309 | −8.136 | 1.00 | 10.91 | C |
| ATOM | 386 | CG | LEU | E | 26 | −11.967 | −10.413 | −7.930 | 1.00 | 11.62 | C |
| ATOM | 387 | CD1 | LEU | E | 26 | −12.480 | −11.774 | −8.397 | 1.00 | 12.37 | C |
| ATOM | 388 | CD2 | LEU | E | 26 | −10.657 | −10.061 | −8.624 | 1.00 | 11.04 | C |
| ATOM | 389 | C | LEU | E | 26 | −14.052 | −9.704 | −5.889 | 1.00 | 10.24 | C |
| ATOM | 390 | O | LEU | E | 26 | −14.154 | −10.852 | −5.438 | 1.00 | 9.91 | O |
| ATOM | 391 | N | VAL | E | 27 | −13.668 | −8.663 | −5.156 | 1.00 | 10.14 | N |
| ATOM | 392 | CA | VAL | E | 27 | −13.332 | −8.791 | −3.735 | 1.00 | 10.34 | C |
| ATOM | 393 | CB | VAL | E | 27 | −12.579 | −7.538 | −3.214 | 1.00 | 10.51 | C |
| ATOM | 394 | CG1 | VAL | E | 27 | −12.321 | −7.634 | −1.707 | 1.00 | 11.71 | C |
| ATOM | 395 | CG2 | VAL | E | 27 | −11.267 | −7.363 | −3.971 | 1.00 | 11.00 | C |
| ATOM | 396 | C | VAL | E | 27 | −14.580 | −9.073 | −2.894 | 1.00 | 10.00 | C |
| ATOM | 397 | O | VAL | E | 27 | −14.551 | −9.921 | −1.999 | 1.00 | 9.83 | O |
| ATOM | 398 | N | VAL | E | 28 | −15.676 | −8.374 | −3.193 | 1.00 | 9.51 | N |
| ATOM | 399 | CA | VAL | E | 28 | −16.957 | −8.612 | −2.523 | 1.00 | 9.32 | C |
| ATOM | 400 | CB | VAL | E | 28 | −18.033 | −7.611 | −3.008 | 1.00 | 9.38 | C |
| ATOM | 401 | CG1 | VAL | E | 28 | −19.404 | −7.945 | −2.425 | 1.00 | 9.42 | C |
| ATOM | 402 | CG2 | VAL | E | 28 | −17.624 | −6.192 | −2.640 | 1.00 | 9.97 | C |
| ATOM | 403 | C | VAL | E | 28 | −17.402 | −10.058 | −2.746 | 1.00 | 8.87 | C |
| ATOM | 404 | O | VAL | E | 28 | −17.747 | −10.760 | −1.795 | 1.00 | 8.20 | O |
| ATOM | 405 | N | ALA | E | 29 | −17.362 | −10.499 | −4.006 | 1.00 | 8.61 | N |
| ATOM | 406 | CA | ALA | E | 29 | −17.701 | −11.875 | −4.362 | 1.00 | 8.75 | C |
| ATOM | 407 | CB | ALA | E | 29 | −17.615 | −12.084 | −5.868 | 1.00 | 8.64 | C |
| ATOM | 408 | C | ALA | E | 29 | −16.824 | −12.890 | −3.632 | 1.00 | 8.64 | C |
| ATOM | 409 | O | ALA | E | 29 | −17.340 | −13.835 | −3.032 | 1.00 | 8.89 | O |
| ATOM | 410 | N | ALA | E | 30 | −15.508 | −12.689 | −3.667 | 1.00 | 8.42 | N |
| ATOM | 411 | CA | ALA | E | 30 | −14.583 | −13.608 | −3.007 | 1.00 | 8.49 | C |
| ATOM | 412 | CB | ALA | E | 30 | −13.131 | −13.223 | −3.300 | 1.00 | 8.86 | C |
| ATOM | 413 | C | ALA | E | 30 | −14.833 | −13.690 | −1.502 | 1.00 | 8.80 | C |
| ATOM | 414 | O | ALA | E | 30 | −14.731 | −14.769 | −0.913 | 1.00 | 8.89 | O |
| ATOM | 415 | N | SER | E | 31 | −15.175 | −12.551 | −0.897 | 1.00 | 8.85 | N |
| ATOM | 416 | CA | SER | E | 31 | −15.470 | −12.487 | 0.534 | 1.00 | 9.12 | C |
| ATOM | 417 | CB | SER | E | 31 | −15.634 | −11.034 | 0.975 | 1.00 | 9.15 | C |
| ATOM | 418 | OG | SER | E | 31 | −14.404 | −10.338 | 0.861 | 1.00 | 9.34 | O |
| ATOM | 419 | C | SER | E | 31 | −16.714 | −13.299 | 0.887 | 1.00 | 9.11 | C |
| ATOM | 420 | O | SER | E | 31 | −16.702 | −14.097 | 1.826 | 1.00 | 9.21 | O |
| ATOM | 421 | N | ILE | E | 32 | −17.777 | −13.103 | 0.111 | 1.00 | 8.79 | N |
| ATOM | 422 | CA | ILE | E | 32 | −19.005 | −13.874 | 0.275 | 1.00 | 9.15 | C |
| ATOM | 423 | CB | ILE | E | 32 | −20.068 | −13.396 | −0.730 | 1.00 | 9.10 | C |
| ATOM | 424 | CG1 | ILE | E | 32 | −20.470 | −11.957 | −0.383 | 1.00 | 9.61 | C |
| ATOM | 425 | CD1 | ILE | E | 32 | −21.163 | −11.207 | −1.502 | 1.00 | 10.44 | C |
| ATOM | 426 | CG2 | ILE | E | 32 | −21.273 | −14.346 | −0.745 | 1.00 | 9.98 | C |
| ATOM | 427 | C | ILE | E | 32 | −18.719 | −15.366 | 0.099 | 1.00 | 9.26 | C |
| ATOM | 428 | O | ILE | E | 32 | −19.174 | −16.202 | 0.886 | 1.00 | 9.30 | O |
| ATOM | 429 | N | ILE | E | 33 | −17.945 | −15.684 | −0.933 | 1.00 | 9.58 | N |
| ATOM | 430 | CA | ILE | E | 33 | −17.578 | −17.071 | −1.239 | 1.00 | 10.37 | C |
| ATOM | 431 | CB | ILE | E | 33 | −16.873 | −17.162 | −2.621 | 1.00 | 10.74 | C |
| ATOM | 432 | CG1 | ILE | E | 33 | −17.892 | −16.943 | −3.758 | 1.00 | 11.46 | C |
| ATOM | 433 | CD1 | ILE | E | 33 | −19.178 | −17.743 | −3.649 | 1.00 | 13.79 | C |
| ATOM | 434 | CG2 | ILE | E | 33 | −16.100 | −18.476 | −2.790 | 1.00 | 11.22 | C |
| ATOM | 435 | C | ILE | E | 33 | −16.756 | −17.723 | −0.125 | 1.00 | 10.50 | C |
| ATOM | 436 | O | ILE | E | 33 | −16.981 | −18.889 | 0.207 | 1.00 | 10.35 | O |
| ATOM | 437 | N | ALA | E | 34 | −15.819 | −16.978 | 0.456 | 1.00 | 10.76 | N |
| ATOM | 438 | CA | ALA | E | 34 | −15.028 | −17.497 | 1.587 | 1.00 | 11.18 | C |
| ATOM | 439 | CB | ALA | E | 34 | −13.995 | −16.473 | 2.048 | 1.00 | 11.33 | C |
| ATOM | 440 | C | ALA | E | 34 | −15.932 | −17.909 | 2.747 | 1.00 | 11.15 | C |
| ATOM | 441 | O | ALA | E | 34 | −15.757 | −18.981 | 3.338 | 1.00 | 11.05 | O |
| ATOM | 442 | N | ILE | E | 35 | −16.907 | −17.057 | 3.055 | 1.00 | 10.80 | N |
| ATOM | 443 | CA | ILE | E | 35 | −17.847 | −17.300 | 4.144 | 1.00 | 10.83 | C |
| ATOM | 444 | CB | ILE | E | 35 | −18.684 | −16.030 | 4.447 | 1.00 | 11.04 | C |
| ATOM | 445 | CG1 | ILE | E | 35 | −17.763 | −14.917 | 4.981 | 1.00 | 10.74 | C |
| ATOM | 446 | CD1 | ILE | E | 35 | −18.346 | −13.512 | 4.949 | 1.00 | 11.96 | C |
| ATOM | 447 | CG2 | ILE | E | 35 | −19.809 | −16.337 | 5.440 | 1.00 | 11.55 | C |
| ATOM | 448 | C | ILE | E | 35 | −18.730 | −18.508 | 3.829 | 1.00 | 10.13 | C |
| ATOM | 449 | O | ILE | E | 35 | −18.883 | −19.416 | 4.661 | 1.00 | 10.09 | O |
| ATOM | 450 | N | LEU | E | 36 | −19.285 | −18.532 | 2.619 | 1.00 | 9.36 | N |
| ATOM | 451 | CA | LEU | E | 36 | −20.065 | −19.677 | 2.163 | 1.00 | 8.87 | C |
| ATOM | 452 | CB | LEU | E | 36 | −20.618 | −19.422 | 0.761 | 1.00 | 8.77 | C |
| ATOM | 453 | CG | LEU | E | 36 | −21.387 | −20.560 | 0.080 | 1.00 | 9.16 | C |
| ATOM | 454 | CD1 | LEU | E | 36 | −22.740 | −20.798 | 0.752 | 1.00 | 9.59 | C |
| ATOM | 455 | CD2 | LEU | E | 36 | −21.566 | −20.258 | −1.403 | 1.00 | 8.86 | C |
| ATOM | 456 | C | LEU | E | 36 | −19.247 | −20.976 | 2.199 | 1.00 | 8.66 | C |
| ATOM | 457 | O | LEU | E | 36 | −19.747 | −22.014 | 2.630 | 1.00 | 8.48 | O |
| ATOM | 458 | N | HIS | E | 37 | −17.995 | −20.914 | 1.755 | 1.00 | 8.53 | N |
| ATOM | 459 | CA | HIS | E | 37 | −17.134 | −22.093 | 1.733 | 1.00 | 8.82 | C |
| ATOM | 460 | CB | HIS | E | 37 | −15.759 | −21.751 | 1.140 | 1.00 | 9.19 | C |
| ATOM | 461 | CG | HIS | E | 37 | −14.921 | −22.949 | 0.807 | 1.00 | 10.39 | C |
| ATOM | 462 | ND1 | HIS | E | 37 | −13.713 | −22.843 | 0.149 | 1.00 | 11.28 | N |
| ATOM | 463 | CE1 | HIS | E | 37 | −13.193 | −24.048 | −0.007 | 1.00 | 11.39 | C |
| ATOM | 464 | NE2 | HIS | E | 37 | −14.026 | −24.934 | 0.509 | 1.00 | 10.36 | N |
| ATOM | 465 | CD2 | HIS | E | 37 | −15.115 | −24.274 | 1.025 | 1.00 | 10.56 | C |
| ATOM | 466 | C | HIS | E | 37 | −16.978 | −22.674 | 3.141 | 1.00 | 8.79 | C |
| ATOM | 467 | O | HIS | E | 37 | −17.123 | −23.886 | 3.338 | 1.00 | 8.35 | O |
| ATOM | 468 | N | LEU | E | 38 | −16.707 | −21.818 | 4.124 | 1.00 | 8.67 | N |
| ATOM | 469 | CA | LEU | E | 38 | −16.577 | −22.301 | 5.502 | 1.00 | 9.33 | C |
| ATOM | 470 | CB | LEU | E | 38 | −16.141 | −21.185 | 6.467 | 1.00 | 9.57 | C |
| ATOM | 471 | CG | LEU | E | 38 | −16.029 | −21.608 | 7.944 | 1.00 | 10.90 | C |
| ATOM | 472 | CD1 | LEU | E | 38 | −14.912 | −22.617 | 8.158 | 1.00 | 11.17 | C |
| ATOM | 473 | CD2 | LEU | E | 38 | −15.872 | −20.435 | 8.893 | 1.00 | 11.25 | C |
| ATOM | 474 | C | LEU | E | 38 | −17.875 | −22.967 | 5.977 | 1.00 | 8.69 | C |
| ATOM | 475 | O | LEU | E | 38 | −17.853 | −24.074 | 6.529 | 1.00 | 8.48 | O |
| ATOM | 476 | N | ILE | E | 39 | −19.005 | −22.300 | 5.749 | 1.00 | 8.51 | N |
| ATOM | 477 | CA | ILE | E | 39 | −20.300 | −22.847 | 6.163 | 1.00 | 8.29 | C |
| ATOM | 478 | CB | ILE | E | 39 | −21.454 | −21.889 | 5.807 | 1.00 | 8.50 | C |
| ATOM | 479 | CG1 | ILE | E | 39 | −21.359 | −20.628 | 6.680 | 1.00 | 8.90 | C |
| ATOM | 480 | CD1 | ILE | E | 39 | −22.233 | −19.464 | 6.215 | 1.00 | 10.20 | C |
| ATOM | 481 | CG2 | ILE | E | 39 | −22.813 | −22.586 | 5.972 | 1.00 | 8.24 | C |
| ATOM | 482 | C | ILE | E | 39 | −20.535 | −24.230 | 5.551 | 1.00 | 8.48 | C |
| ATOM | 483 | O | ILE | E | 39 | −20.856 | −25.176 | 6.262 | 1.00 | 7.96 | O |
| ATOM | 484 | N | LEU | E | 40 | −20.361 | −24.336 | 4.235 | 1.00 | 8.29 | N |
| ATOM | 485 | CA | LEU | E | 40 | −20.570 | −25.600 | 3.527 | 1.00 | 8.55 | C |
| ATOM | 486 | CB | LEU | E | 40 | −20.402 | −25.412 | 2.019 | 1.00 | 8.58 | C |
| ATOM | 487 | CG | LEU | E | 40 | −21.399 | −24.490 | 1.323 | 1.00 | 8.81 | C |
| ATOM | 488 | CD1 | LEU | E | 40 | −21.040 | −24.367 | −0.156 | 1.00 | 8.98 | C |
| ATOM | 489 | CD2 | LEU | E | 40 | −22.830 | −25.001 | 1.505 | 1.00 | 9.77 | C |
| ATOM | 490 | C | LEU | E | 40 | −19.628 | −26.691 | 4.015 | 1.00 | 8.56 | C |
| ATOM | 491 | O | LEU | E | 40 | −20.036 | −27.845 | 4.186 | 1.00 | 8.81 | O |
| ATOM | 492 | N | TRP | E | 41 | −18.370 | −26.319 | 4.241 | 1.00 | 8.83 | N |
| ATOM | 493 | CA | TRP | E | 41 | −17.364 | −27.279 | 4.679 | 1.00 | 9.18 | C |
| ATOM | 494 | CB | TRP | E | 41 | −15.959 | −26.680 | 4.636 | 1.00 | 9.61 | C |
| ATOM | 495 | CG | TRP | E | 41 | −14.903 | −27.630 | 5.104 | 1.00 | 10.09 | C |
| ATOM | 496 | CD1 | TRP | E | 41 | −14.347 | −28.659 | 4.393 | 1.00 | 10.64 | C |
| ATOM | 497 | NE1 | TRP | E | 41 | −13.402 | −29.309 | 5.159 | 1.00 | 10.16 | N |
| ATOM | 498 | CE2 | TRP | E | 41 | −13.340 | −28.705 | 6.389 | 1.00 | 10.15 | C |
| ATOM | 499 | CD2 | TRP | E | 41 | −14.273 | −27.644 | 6.391 | 1.00 | 10.02 | C |
| ATOM | 500 | CE3 | TRP | E | 41 | −14.406 | −26.859 | 7.547 | 1.00 | 10.49 | C |
| ATOM | 501 | CZ3 | TRP | E | 41 | −13.613 | −27.159 | 8.653 | 1.00 | 10.61 | C |
| ATOM | 502 | CH2 | TRP | E | 41 | −12.695 | −28.225 | 8.618 | 1.00 | 10.07 | C |
| ATOM | 503 | CZ2 | TRP | E | 41 | −12.546 | −29.007 | 7.501 | 1.00 | 9.78 | C |
| ATOM | 504 | C | TRP | E | 41 | −17.687 | −27.819 | 6.069 | 1.00 | 9.33 | C |
| ATOM | 505 | O | TRP | E | 41 | −17.602 | −29.021 | 6.293 | 1.00 | 9.15 | O |
| ATOM | 506 | N | ILE | E | 42 | −18.087 | −26.941 | 6.986 | 1.00 | 9.77 | N |
| ATOM | 507 | CA | ILE | E | 42 | −18.511 | −27.385 | 8.321 | 1.00 | 10.12 | C |
| ATOM | 508 | CB | ILE | E | 42 | −18.831 | −26.195 | 9.262 | 1.00 | 10.52 | C |
| ATOM | 509 | CG1 | ILE | E | 42 | −17.548 | −25.416 | 9.571 | 1.00 | 9.76 | C |
| ATOM | 510 | CD1 | ILE | E | 42 | −17.768 | −24.091 | 10.297 | 1.00 | 10.31 | C |
| ATOM | 511 | CG2 | ILE | E | 42 | −19.465 | −26.685 | 10.575 | 1.00 | 10.93 | C |
| ATOM | 512 | C | ILE | E | 42 | −19.694 | −28.352 | 8.200 | 1.00 | 10.37 | C |
| ATOM | 513 | O | ILE | E | 42 | −19.681 | −29.435 | 8.796 | 1.00 | 10.36 | O |
| ATOM | 514 | N | LEU | E | 43 | −20.691 | −27.977 | 7.400 | 1.00 | 10.27 | N |
| ATOM | 515 | CA | LEU | E | 43 | −21.878 | −28.824 | 7.205 | 1.00 | 10.73 | C |
| ATOM | 516 | CB | LEU | E | 43 | −22.929 | −28.101 | 6.362 | 1.00 | 10.48 | C |
| ATOM | 517 | CG | LEU | E | 43 | −23.571 | −26.892 | 7.044 | 1.00 | 9.60 | C |
| ATOM | 518 | CD1 | LEU | E | 43 | −24.331 | −26.039 | 6.034 | 1.00 | 9.26 | C |
| ATOM | 519 | CD2 | LEU | E | 43 | −24.483 | −27.312 | 8.207 | 1.00 | 10.52 | C |
| ATOM | 520 | C | LEU | E | 43 | −21.528 | −30.180 | 6.597 | 1.00 | 11.49 | C |
| ATOM | 521 | O | LEU | E | 43 | −22.091 | −31.207 | 6.988 | 1.00 | 11.96 | O |
| ATOM | 522 | N | ASP | E | 44 | −20.583 | −30.175 | 5.658 | 1.00 | 12.33 | N |
| ATOM | 523 | CA | ASP | E | 44 | −20.088 | −31.401 | 5.044 | 1.00 | 13.62 | C |
| ATOM | 524 | CB | ASP | E | 44 | −19.134 | −31.080 | 3.892 | 1.00 | 14.16 | C |
| ATOM | 525 | CG | ASP | E | 44 | −18.526 | −32.325 | 3.277 | 1.00 | 15.96 | C |
| ATOM | 526 | OD1 | ASP | E | 44 | −19.266 | −33.098 | 2.636 | 1.00 | 19.34 | O |
| ATOM | 527 | OD2 | ASP | E | 44 | −17.310 | −32.535 | 3.455 | 1.00 | 19.79 | O |
| ATOM | 528 | C | ASP | E | 44 | −19.399 | −32.311 | 6.067 | 1.00 | 13.94 | C |
| ATOM | 529 | O | ASP | E | 44 | −19.623 | −33.525 | 6.069 | 1.00 | 14.14 | O |
| ATOM | 530 | N | ARG | E | 45 | −18.574 | −31.728 | 6.936 | 1.00 | 14.61 | N |
| ATOM | 531 | CA | ARG | E | 45 | −17.857 | −32.518 | 7.938 | 1.00 | 15.30 | C |
| ATOM | 532 | CB | ARG | E | 45 | −16.724 | −31.720 | 8.598 | 1.00 | 15.51 | C |
| ATOM | 533 | CG | ARG | E | 45 | −15.674 | −31.170 | 7.629 | 1.00 | 16.51 | C |
| ATOM | 534 | CD | ARG | E | 45 | −15.256 | −32.191 | 6.570 | 1.00 | 19.43 | C |
| ATOM | 535 | NE | ARG | E | 45 | −14.427 | −33.257 | 7.119 | 1.00 | 23.11 | N |
| ATOM | 536 | CZ | ARG | E | 45 | −14.252 | −34.446 | 6.547 | 1.00 | 24.93 | C |
| ATOM | 537 | NH1 | ARG | E | 45 | −14.865 | −34.738 | 5.404 | 1.00 | 26.08 | N |
| ATOM | 538 | NH2 | ARG | E | 45 | −13.477 | −35.353 | 7.130 | 1.00 | 26.17 | N |
| ATOM | 539 | C | ARG | E | 45 | −18.800 | −33.100 | 8.991 | 1.00 | 15.49 | C |
| ATOM | 540 | O | ARG | E | 45 | −18.577 | −34.207 | 9.478 | 1.00 | 15.92 | O |
| ATOM | 541 | N | LEU | E | 46 | −19.854 | −32.359 | 9.323 | 1.00 | 15.62 | N |
| ATOM | 542 | CA | LEU | E | 46 | −20.865 | −32.840 | 10.273 | 1.00 | 15.62 | C |
| ATOM | 543 | CB | LEU | E | 46 | −21.742 | −31.685 | 10.771 | 1.00 | 15.53 | C |
| ATOM | 544 | CG | LEU | E | 46 | −21.119 | −30.613 | 11.675 | 1.00 | 15.60 | C |
| ATOM | 545 | CD1 | LEU | E | 46 | −22.164 | −29.557 | 12.007 | 1.00 | 15.21 | C |
| ATOM | 546 | CD2 | LEU | E | 46 | −20.532 | −31.210 | 12.961 | 1.00 | 15.05 | C |
| ATOM | 547 | C | LEU | E | 46 | −21.732 | −33.939 | 9.667 | 1.00 | 15.77 | C |
| ATOM | 548 | O | LEU | E | 46 | −22.325 | −34.743 | 10.394 | 1.00 | 16.43 | O |
| ATOM | 549 | N | NH2 | E | 47 | −21.756 | −34.219 | 8.437 | 1.00 | 15.32 | N |
| ATOM | 550 | BR4 | BRB | F | 1 | −3.503 | −0.725 | −3.470 | 1.00 | 35.57 | BR |
| ATOM | 551 | C4 | BRB | F | 1 | −3.408 | −2.411 | −4.324 | 1.00 | 29.89 | C |
| ATOM | 552 | C3 | BRB | F | 1 | −4.543 | −2.972 | −4.909 | 1.00 | 30.70 | C |
| ATOM | 553 | C2 | BRB | F | 1 | −4.460 | −4.215 | −5.536 | 1.00 | 30.42 | C |
| ATOM | 554 | C5 | BRB | F | 1 | −2.191 | −3.085 | −4.363 | 1.00 | 30.51 | C |
| ATOM | 555 | C6 | BRB | F | 1 | −2.109 | −4.326 | −4.987 | 1.00 | 30.02 | C |
| ATOM | 556 | C1 | BRB | F | 1 | −3.240 | −4.891 | −5.575 | 1.00 | 28.94 | C |
| ATOM | 557 | C7 | BRB | F | 1 | −3.136 | −6.241 | −6.256 | 1.00 | 23.05 | C |
| ATOM | 558 | O1 | BRB | F | 1 | −4.201 | −7.071 | −5.786 | 1.00 | 18.92 | O |
| ATOM | 559 | N | PRO | F | 25 | −2.223 | −6.806 | −6.935 | 1.00 | 16.43 | N |
| ATOM | 560 | CA | PRO | F | 25 | −2.295 | −8.153 | −7.486 | 1.00 | 14.44 | C |
| ATOM | 561 | CB | PRO | F | 25 | −0.967 | −8.284 | −8.239 | 1.00 | 14.46 | C |
| ATOM | 562 | CG | PRO | F | 25 | −0.726 | −6.902 | −8.744 | 1.00 | 15.36 | C |
| ATOM | 563 | CD | PRO | F | 25 | −1.238 | −5.981 | −7.664 | 1.00 | 15.49 | C |
| ATOM | 564 | C | PRO | F | 25 | −2.432 | −9.259 | −6.441 | 1.00 | 13.51 | C |
| ATOM | 565 | O | PRO | F | 25 | −3.033 | −10.286 | −6.738 | 1.00 | 13.09 | O |
| ATOM | 566 | N | LEU | F | 26 | −1.890 | −9.067 | −5.239 | 1.00 | 12.44 | N |
| ATOM | 567 | CA | LEU | F | 26 | −2.027 | −10.092 | −4.202 | 1.00 | 12.06 | C |
| ATOM | 568 | CB | LEU | F | 26 | −1.236 | −9.733 | −2.939 | 1.00 | 11.95 | C |
| ATOM | 569 | CG | LEU | F | 26 | −1.500 | −10.574 | −1.680 | 1.00 | 11.92 | C |
| ATOM | 570 | CD1 | LEU | F | 26 | −1.109 | −12.038 | −1.864 | 1.00 | 12.16 | C |
| ATOM | 571 | CD2 | LEU | F | 26 | −0.785 | −9.981 | −0.473 | 1.00 | 12.46 | C |
| ATOM | 572 | C | LEU | F | 26 | −3.499 | −10.326 | −3.860 | 1.00 | 11.71 | C |
| ATOM | 573 | O | LEU | F | 26 | −3.952 | −11.472 | −3.778 | 1.00 | 11.87 | O |
| ATOM | 574 | N | VAL | F | 27 | −4.233 | −9.234 | −3.664 | 1.00 | 11.58 | N |
| ATOM | 575 | CA | VAL | F | 27 | −5.656 | −9.308 | −3.315 | 1.00 | 11.73 | C |
| ATOM | 576 | CB | VAL | F | 27 | −6.190 | −7.937 | −2.826 | 1.00 | 12.17 | C |
| ATOM | 577 | CG1 | VAL | F | 27 | −7.683 | −8.012 | −2.519 | 1.00 | 13.19 | C |
| ATOM | 578 | CG2 | VAL | F | 27 | −5.423 | −7.491 | −1.587 | 1.00 | 13.00 | C |
| ATOM | 579 | C | VAL | F | 27 | −6.495 | −9.863 | −4.476 | 1.00 | 11.27 | C |
| ATOM | 580 | O | VAL | F | 27 | −7.382 | −10.700 | −4.261 | 1.00 | 11.23 | O |
| ATOM | 581 | N | VAL | F | 28 | −6.202 | −9.414 | −5.699 | 1.00 | 10.62 | N |
| ATOM | 582 | CA | VAL | F | 28 | −6.865 | −9.938 | −6.902 | 1.00 | 9.92 | C |
| ATOM | 583 | CB | VAL | F | 28 | −6.365 | −9.216 | −8.193 | 1.00 | 10.14 | C |
| ATOM | 584 | CG1 | VAL | F | 28 | −6.937 | −9.865 | −9.457 | 1.00 | 10.40 | C |
| ATOM | 585 | CG2 | VAL | F | 28 | −6.724 | −7.735 | −8.133 | 1.00 | 10.52 | C |
| ATOM | 586 | C | VAL | F | 28 | −6.643 | −11.447 | −6.996 | 1.00 | 9.60 | C |
| ATOM | 587 | O | VAL | F | 28 | −7.600 | −12.219 | −7.111 | 1.00 | 9.14 | O |
| ATOM | 588 | N | ALA | F | 29 | −5.378 | −11.863 | −6.914 | 1.00 | 9.10 | N |
| ATOM | 589 | CA | ALA | F | 29 | −5.033 | −13.279 | −7.006 | 1.00 | 8.65 | C |
| ATOM | 590 | CB | ALA | F | 29 | −3.522 | −13.467 | −6.990 | 1.00 | 8.83 | C |
| ATOM | 591 | C | ALA | F | 29 | −5.702 | −14.120 | −5.914 | 1.00 | 8.48 | C |
| ATOM | 592 | O | ALA | F | 29 | −6.228 | −15.196 | −6.204 | 1.00 | 8.21 | O |
| ATOM | 593 | N | ALA | F | 30 | −5.710 | −13.620 | −4.678 | 1.00 | 8.36 | N |
| ATOM | 594 | CA | ALA | F | 30 | −6.324 | −14.352 | −3.563 | 1.00 | 8.24 | C |
| ATOM | 595 | CB | ALA | F | 30 | −6.031 | −13.662 | −2.238 | 1.00 | 8.95 | C |
| ATOM | 596 | C | ALA | F | 30 | −7.830 | −14.497 | −3.773 | 1.00 | 8.40 | C |
| ATOM | 597 | O | ALA | F | 30 | −8.419 | −15.535 | −3.454 | 1.00 | 8.52 | O |
| ATOM | 598 | N | SER | F | 31 | −8.440 | −13.444 | −4.313 | 1.00 | 7.96 | N |
| ATOM | 599 | CA | SER | F | 31 | −9.866 | −13.434 | −4.607 | 1.00 | 8.29 | C |
| ATOM | 600 | CB | SER | F | 31 | −10.309 | −12.030 | −5.021 | 1.00 | 8.37 | C |
| ATOM | 601 | OG | SER | F | 31 | −10.115 | −11.132 | −3.938 | 1.00 | 9.22 | O |
| ATOM | 602 | C | SER | F | 31 | −10.206 | −14.461 | −5.681 | 1.00 | 8.14 | C |
| ATOM | 603 | O | SER | F | 31 | −11.138 | −15.251 | −5.525 | 1.00 | 8.19 | O |
| ATOM | 604 | N | ILE | F | 32 | −9.428 | −14.469 | −6.761 | 1.00 | 8.08 | N |
| ATOM | 605 | CA | ILE | F | 32 | −9.591 | −15.473 | −7.811 | 1.00 | 7.91 | C |
| ATOM | 606 | CB | ILE | F | 32 | −8.562 | −15.253 | −8.940 | 1.00 | 8.27 | C |
| ATOM | 607 | CG1 | ILE | F | 32 | −8.847 | −13.915 | −9.630 | 1.00 | 8.22 | C |
| ATOM | 608 | CD1 | ILE | F | 32 | −7.739 | −13.438 | −10.552 | 1.00 | 9.18 | C |
| ATOM | 609 | CG2 | ILE | F | 32 | −8.571 | −16.418 | −9.935 | 1.00 | 7.39 | C |
| ATOM | 610 | C | ILE | F | 32 | −9.457 | −16.877 | −7.225 | 1.00 | 8.51 | C |
| ATOM | 611 | O | ILE | F | 32 | −10.267 | −17.765 | −7.506 | 1.00 | 8.04 | O |
| ATOM | 612 | N | ILE | F | 33 | −8.432 | −17.060 | −6.397 | 1.00 | 8.30 | N |
| ATOM | 613 | CA | ILE | F | 33 | −8.122 | −18.363 | −5.813 | 1.00 | 9.14 | C |
| ATOM | 614 | CB | ILE | F | 33 | −6.703 | −18.362 | −5.186 | 1.00 | 9.32 | C |
| ATOM | 615 | CG1 | ILE | F | 33 | −5.680 | −18.581 | −6.310 | 1.00 | 10.54 | C |
| ATOM | 616 | CD1 | ILE | F | 33 | −4.283 | −18.064 | −6.033 | 1.00 | 13.15 | C |
| ATOM | 617 | CG2 | ILE | F | 33 | −6.559 | −19.442 | −4.126 | 1.00 | 10.10 | C |
| ATOM | 618 | C | ILE | F | 33 | −9.206 | −18.838 | −4.849 | 1.00 | 9.40 | C |
| ATOM | 619 | O | ILE | F | 33 | −9.549 | −20.024 | −4.837 | 1.00 | 9.18 | O |
| ATOM | 620 | N | ALA | F | 34 | −9.767 | −17.910 | −4.074 | 1.00 | 9.58 | N |
| ATOM | 621 | CA | ALA | F | 34 | −10.885 | −18.240 | −3.177 | 1.00 | 9.81 | C |
| ATOM | 622 | CB | ALA | F | 34 | −11.294 | −17.031 | −2.347 | 1.00 | 10.17 | C |
| ATOM | 623 | C | ALA | F | 34 | −12.085 | −18.792 | −3.957 | 1.00 | 9.74 | C |
| ATOM | 624 | O | ALA | F | 34 | −12.709 | −19.778 | −3.543 | 1.00 | 9.92 | O |
| ATOM | 625 | N | ILE | F | 35 | −12.397 | −18.160 | −5.090 | 1.00 | 9.19 | N |
| ATOM | 626 | CA | ILE | F | 35 | −13.501 | −18.606 | −5.943 | 1.00 | 8.89 | C |
| ATOM | 627 | CB | ILE | F | 35 | −13.847 | −17.557 | −7.036 | 1.00 | 8.92 | C |
| ATOM | 628 | CG1 | ILE | F | 35 | −14.292 | −16.242 | −6.373 | 1.00 | 9.56 | C |
| ATOM | 629 | CD1 | ILE | F | 35 | −14.464 | −15.070 | −7.319 | 1.00 | 9.26 | C |
| ATOM | 630 | CG2 | ILE | F | 35 | −14.920 | −18.099 | −7.996 | 1.00 | 8.87 | C |
| ATOM | 631 | C | ILE | F | 35 | −13.171 | −19.967 | −6.557 | 1.00 | 8.57 | C |
| ATOM | 632 | O | ILE | F | 35 | −13.989 | −20.883 | −6.513 | 1.00 | 8.29 | O |
| ATOM | 633 | N | LEU | F | 36 | −11.960 | −20.101 | −7.097 | 1.00 | 8.08 | N |
| ATOM | 634 | CA | LEU | F | 36 | −11.514 | −21.375 | −7.663 | 1.00 | 8.43 | C |
| ATOM | 635 | CB | LEU | F | 36 | −10.105 | −21.245 | −8.243 | 1.00 | 8.53 | C |
| ATOM | 636 | CG | LEU | F | 36 | −9.502 | −22.523 | −8.831 | 1.00 | 8.44 | C |
| ATOM | 637 | CD1 | LEU | F | 36 | −10.370 | −23.087 | −9.960 | 1.00 | 9.84 | C |
| ATOM | 638 | CD2 | LEU | F | 36 | −8.077 | −22.245 | −9.315 | 1.00 | 9.69 | C |
| ATOM | 639 | C | LEU | F | 36 | −11.560 | −22.499 | −6.625 | 1.00 | 8.23 | C |
| ATOM | 640 | O | LEU | F | 36 | −12.025 | −23.602 | −6.911 | 1.00 | 7.94 | O |
| ATOM | 641 | N | HIS | F | 37 | −11.075 | −22.209 | −5.423 | 1.00 | 8.40 | N |
| ATOM | 642 | CA | HIS | F | 37 | −11.036 | −23.208 | −4.353 | 1.00 | 8.66 | C |
| ATOM | 643 | CB | HIS | F | 37 | −10.407 | −22.610 | −3.092 | 1.00 | 8.71 | C |
| ATOM | 644 | CG | HIS | F | 37 | −10.077 | −23.619 | −2.037 | 1.00 | 9.01 | C |
| ATOM | 645 | ND1 | HIS | F | 37 | −9.395 | −23.282 | −0.888 | 1.00 | 9.93 | N |
| ATOM | 646 | CE1 | HIS | F | 37 | −9.244 | −24.358 | −0.136 | 1.00 | 10.45 | C |
| ATOM | 647 | NE2 | HIS | F | 37 | −9.793 | −25.384 | −0.761 | 1.00 | 9.92 | N |
| ATOM | 648 | CD2 | HIS | F | 37 | −10.322 | −24.950 | −1.952 | 1.00 | 9.79 | C |
| ATOM | 649 | C | HIS | F | 37 | −12.428 | −23.767 | −4.038 | 1.00 | 8.77 | C |
| ATOM | 650 | O | HIS | F | 37 | −12.591 | −24.977 | −3.898 | 1.00 | 9.18 | O |
| ATOM | 651 | N | LEU | F | 38 | −13.430 | −22.898 | −3.932 | 1.00 | 9.25 | N |
| ATOM | 652 | CA | LEU | F | 38 | −14.799 | −23.363 | −3.685 | 1.00 | 9.37 | C |
| ATOM | 653 | CB | LEU | F | 38 | −15.753 | −22.201 | −3.403 | 1.00 | 9.36 | C |
| ATOM | 654 | CG | LEU | F | 38 | −17.201 | −22.638 | −3.116 | 1.00 | 10.42 | C |
| ATOM | 655 | CD1 | LEU | F | 38 | −17.288 | −23.640 | −1.962 | 1.00 | 12.68 | C |
| ATOM | 656 | CD2 | LEU | F | 38 | −18.083 | −21.454 | −2.832 | 1.00 | 13.16 | C |
| ATOM | 657 | C | LEU | F | 38 | −15.326 | −24.215 | −4.841 | 1.00 | 9.41 | C |
| ATOM | 658 | O | LEU | F | 38 | −15.911 | −25.278 | −4.616 | 1.00 | 9.35 | O |
| ATOM | 659 | N | ILE | F | 39 | −15.113 | −23.754 | −6.072 | 1.00 | 9.38 | N |
| ATOM | 660 | CA | ILE | F | 39 | −15.537 | −24.513 | −7.256 | 1.00 | 9.82 | C |
| ATOM | 661 | CB | ILE | F | 39 | −15.171 | −23.780 | −8.571 | 1.00 | 9.74 | C |
| ATOM | 662 | CG1 | ILE | F | 39 | −15.987 | −22.481 | −8.693 | 1.00 | 10.19 | C |
| ATOM | 663 | CD1 | ILE | F | 39 | −15.549 | −21.544 | −9.835 | 1.00 | 10.41 | C |
| ATOM | 664 | CG2 | ILE | F | 39 | −15.375 | −24.707 | −9.786 | 1.00 | 10.22 | C |
| ATOM | 665 | C | ILE | F | 39 | −14.932 | −25.921 | −7.233 | 1.00 | 9.94 | C |
| ATOM | 666 | O | ILE | F | 39 | −15.645 | −26.918 | −7.374 | 1.00 | 10.10 | O |
| ATOM | 667 | N | LEU | F | 40 | −13.621 | −25.996 | −7.015 | 1.00 | 10.01 | N |
| ATOM | 668 | CA | LEU | F | 40 | −12.933 | −27.281 | −6.955 | 1.00 | 10.38 | C |
| ATOM | 669 | CB | LEU | F | 40 | −11.431 | −27.071 | −6.797 | 1.00 | 9.94 | C |
| ATOM | 670 | CG | LEU | F | 40 | −10.657 | −26.532 | −7.993 | 1.00 | 10.49 | C |
| ATOM | 671 | CD1 | LEU | F | 40 | −9.243 | −26.238 | −7.539 | 1.00 | 10.49 | C |
| ATOM | 672 | CD2 | LEU | F | 40 | −10.672 | −27.533 | −9.155 | 1.00 | 10.98 | C |
| ATOM | 673 | C | LEU | F | 40 | −13.448 | −28.164 | −5.824 | 1.00 | 11.05 | C |
| ATOM | 674 | O | LEU | F | 40 | −13.642 | −29.368 | −6.011 | 1.00 | 11.71 | O |
| ATOM | 675 | N | TRP | F | 41 | −13.680 | −27.565 | −4.657 | 1.00 | 11.36 | N |
| ATOM | 676 | CA | TRP | F | 41 | −14.108 | −28.333 | −3.496 | 1.00 | 12.50 | C |
| ATOM | 677 | CB | TRP | F | 41 | −14.009 | −27.500 | −2.210 | 1.00 | 12.53 | C |
| ATOM | 678 | CG | TRP | F | 41 | −14.476 | −28.251 | −1.009 | 1.00 | 12.82 | C |
| ATOM | 679 | CD1 | TRP | F | 41 | −13.773 | −29.181 | −0.296 | 1.00 | 12.95 | C |
| ATOM | 680 | NE1 | TRP | F | 41 | −14.543 | −29.671 | 0.732 | 1.00 | 12.54 | N |
| ATOM | 681 | CE2 | TRP | F | 41 | −15.772 | −29.062 | 0.693 | 1.00 | 12.81 | C |
| ATOM | 682 | CD2 | TRP | F | 41 | −15.766 | −28.163 | −0.396 | 1.00 | 12.73 | C |
| ATOM | 683 | CE3 | TRP | F | 41 | −16.916 | −27.405 | −0.658 | 1.00 | 13.36 | C |
| ATOM | 684 | CZ3 | TRP | F | 41 | −18.024 | −27.568 | 0.166 | 1.00 | 13.17 | C |
| ATOM | 685 | CH2 | TRP | F | 41 | −18.000 | −28.473 | 1.244 | 1.00 | 13.16 | C |
| ATOM | 686 | CZ2 | TRP | F | 41 | −16.890 | −29.227 | 1.524 | 1.00 | 13.20 | C |
| ATOM | 687 | C | TRP | F | 41 | −15.512 | −28.918 | −3.696 | 1.00 | 12.86 | C |
| ATOM | 688 | O | TRP | F | 41 | −15.741 | −30.097 | −3.414 | 1.00 | 13.40 | O |
| ATOM | 689 | N | ILE | F | 42 | −16.437 | −28.106 | −4.203 | 1.00 | 13.75 | N |
| ATOM | 690 | CA | ILE | F | 42 | −17.794 | −28.588 | −4.509 | 1.00 | 14.53 | C |
| ATOM | 691 | CB | ILE | F | 42 | −18.710 | −27.465 | −5.048 | 1.00 | 14.50 | C |
| ATOM | 692 | CG1 | ILE | F | 42 | −18.967 | −26.421 | −3.957 | 1.00 | 14.31 | C |
| ATOM | 693 | CD1 | ILE | F | 42 | −19.695 | −25.164 | −4.435 | 1.00 | 14.38 | C |
| ATOM | 694 | CG2 | ILE | F | 42 | −20.043 | −28.044 | −5.546 | 1.00 | 14.72 | C |
| ATOM | 695 | C | ILE | F | 42 | −17.733 | −29.760 | −5.495 | 1.00 | 15.34 | C |
| ATOM | 696 | O | ILE | F | 42 | −18.350 | −30.801 | −5.266 | 1.00 | 15.61 | O |
| ATOM | 697 | N | LEU | F | 43 | −16.968 | −29.590 | −6.573 | 1.00 | 16.26 | N |
| ATOM | 698 | CA | LEU | F | 43 | −16.818 | −30.641 | −7.583 | 1.00 | 17.66 | C |
| ATOM | 699 | CB | LEU | F | 43 | −16.045 | −30.125 | −8.802 | 1.00 | 17.73 | C |
| ATOM | 700 | CG | LEU | F | 43 | −16.781 | −29.089 | −9.655 | 1.00 | 18.06 | C |
| ATOM | 701 | CD1 | LEU | F | 43 | −15.850 | −28.506 | −10.720 | 1.00 | 17.90 | C |
| ATOM | 702 | CD2 | LEU | F | 43 | −18.040 | −29.673 | −10.296 | 1.00 | 19.06 | C |
| ATOM | 703 | C | LEU | F | 43 | −16.173 | −31.909 | −7.027 | 1.00 | 18.56 | C |
| ATOM | 704 | O | LEU | F | 43 | −16.581 | −33.022 | −7.379 | 1.00 | 18.74 | O |
| ATOM | 705 | N | ASP | F | 44 | −15.174 | −31.738 | −6.162 | 1.00 | 19.54 | N |
| ATOM | 706 | CA | ASP | F | 44 | −14.524 | −32.860 | −5.480 | 1.00 | 20.87 | C |
| ATOM | 707 | CB | ASP | F | 44 | −13.371 | −32.368 | −4.600 | 1.00 | 20.88 | C |
| ATOM | 708 | CG | ASP | F | 44 | −12.616 | −33.507 | −3.932 | 1.00 | 21.83 | C |
| ATOM | 709 | OD1 | ASP | F | 44 | −12.027 | −34.340 | −4.658 | 1.00 | 23.10 | O |
| ATOM | 710 | OD2 | ASP | F | 44 | −12.606 | −33.570 | −2.681 | 1.00 | 22.41 | O |
| ATOM | 711 | C | ASP | F | 44 | −15.507 | −33.660 | −4.627 | 1.00 | 21.71 | C |
| ATOM | 712 | O | ASP | F | 44 | −15.486 | −34.892 | −4.639 | 1.00 | 21.64 | O |
| ATOM | 713 | N | ARG | F | 45 | −16.359 | −32.957 | −3.885 | 1.00 | 22.93 | N |
| ATOM | 714 | CA | ARG | F | 45 | −17.347 | −33.611 | −3.027 | 1.00 | 24.33 | C |
| ATOM | 715 | CB | ARG | F | 45 | −17.941 | −32.623 | −2.015 | 1.00 | 24.52 | C |
| ATOM | 716 | CG | ARG | F | 45 | −16.923 | −32.047 | −1.022 | 1.00 | 25.34 | C |
| ATOM | 717 | CD | ARG | F | 45 | −16.079 | −33.133 | −0.353 | 1.00 | 27.38 | C |
| ATOM | 718 | NE | ARG | F | 45 | −16.824 | −33.867 | 0.670 | 1.00 | 29.22 | N |
| ATOM | 719 | CZ | ARG | F | 45 | −16.388 | −34.967 | 1.280 | 1.00 | 30.72 | C |
| ATOM | 720 | NH1 | ARG | F | 45 | −15.206 | −35.487 | 0.972 | 1.00 | 31.47 | N |
| ATOM | 721 | NH2 | ARG | F | 45 | −17.139 | −35.558 | 2.201 | 1.00 | 31.41 | N |
| ATOM | 722 | C | ARG | F | 45 | −18.444 | −34.305 | −3.837 | 1.00 | 25.22 | C |
| ATOM | 723 | O | ARG | F | 45 | −18.814 | −35.440 | −3.533 | 1.00 | 25.53 | O |
| ATOM | 724 | N | LEU | F | 46 | −18.946 | −33.629 | −4.868 | 1.00 | 26.14 | N |
| ATOM | 725 | CA | LEU | F | 46 | −19.954 | −34.212 | −5.755 | 1.00 | 27.06 | C |
| ATOM | 726 | CB | LEU | F | 46 | −20.749 | −33.117 | −6.475 | 1.00 | 27.26 | C |
| ATOM | 727 | CG | LEU | F | 46 | −21.814 | −32.358 | −5.679 | 1.00 | 28.10 | C |
| ATOM | 728 | CD1 | LEU | F | 46 | −22.471 | −31.297 | −6.551 | 1.00 | 28.66 | C |
| ATOM | 729 | CD2 | LEU | F | 46 | −22.866 | −33.302 | −5.105 | 1.00 | 28.85 | C |
| ATOM | 730 | C | LEU | F | 46 | −19.336 | −35.170 | −6.770 | 1.00 | 27.54 | C |
| ATOM | 731 | O | LEU | F | 46 | −19.966 | −36.154 | −7.172 | 1.00 | 28.04 | O |
| ATOM | 732 | N | NH2 | F | 47 | −18.136 | −35.531 | −6.918 | 1.00 | 27.90 | N |
| ATOM | 733 | O | HOH | B | 1 | −13.149 | −19.835 | 3.566 | 1.00 | 12.27 | O |
| ATOM | 734 | O | HOH | B | 2 | −23.375 | −36.467 | 7.152 | 1.00 | 24.96 | O |
| ATOM | 735 | O | HOH | B | 3 | −12.875 | −20.281 | −0.901 | 1.00 | 13.90 | O |
| ATOM | 736 | O | HOH | B | 4 | −8.671 | −20.203 | 3.859 | 1.00 | 16.70 | O |
| ATOM | 737 | O | HOH | B | 5 | −8.430 | −20.577 | −0.593 | 1.00 | 14.82 | O |
| ATOM | 738 | O | HOH | B | 6 | −14.390 | −6.619 | −9.898 | 1.00 | 12.72 | O |
| ATOM | 739 | O | HOH | B | 7 | −5.588 | −4.990 | 9.754 | 1.00 | 21.25 | O |
| ATOM | 740 | O | HOH | B | 9 | −20.473 | −35.633 | 4.087 | 1.00 | 43.82 | O |
| ATOM | 741 | O | HOH | B | 10 | −11.758 | −31.725 | 5.016 | 1.00 | 17.86 | O |
| ATOM | 742 | O | HOH | B | 11 | −23.850 | −32.197 | 4.881 | 1.00 | 12.59 | O |
| ATOM | 743 | O | HOH | B | 12 | −6.152 | −38.192 | 9.157 | 1.00 | 50.32 | O |
| ATOM | 744 | O | HOH | B | 13 | −9.397 | −32.131 | 2.353 | 1.00 | 18.66 | O |
| ATOM | 745 | O | HOH | B | 14 | −14.338 | −31.643 | 2.835 | 1.00 | 21.22 | O |
| ATOM | 746 | O | HOH | B | 15 | −12.138 | −32.112 | 0.083 | 1.00 | 20.60 | O |
| ATOM | 747 | O | HOH | B | 17 | −12.675 | −26.659 | 2.644 | 1.00 | 26.13 | O |
| ATOM | 748 | O | HOH | B | 18 | −10.676 | −36.259 | 9.305 | 1.00 | 35.14 | O |
| ATOM | 749 | O | HOH | B | 19 | −19.351 | −4.620 | 4.363 | 1.00 | 22.44 | O |
| ATOM | 750 | O | HOH | B | 23 | −12.049 | −33.261 | 2.780 | 1.00 | 50.27 | O |
| ATOM | 751 | O | HOH | B | 25 | −17.854 | −33.483 | −9.967 | 1.00 | 39.02 | O |
| ATOM | 752 | O | AHOH | B | 28 | −11.980 | −18.899 | 1.282 | 0.60 | 11.76 | O |
| ATOM | 753 | O | BHOH | B | 32 | −10.823 | −18.897 | 2.608 | 0.40 | 8.95 | O |
| ATOM | 754 | O | AHOH | B | 30 | −9.325 | −19.029 | 1.555 | 0.60 | 9.84 | O |
| ATOM | 755 | O | BHOH | B | 33 | −10.461 | −18.960 | 0.168 | 0.40 | 10.25 | O |
| ATOM | 756 | O | HOH | B | 29 | −20.080 | −1.789 | 3.369 | 1.00 | 41.73 | O |
| ATOM | 757 | O | HOH | B | 31 | −10.075 | −27.002 | 2.012 | 1.00 | 32.23 | O |
| ATOM | 758 | O2 | IPA | I | 1 | −28.432 | −23.509 | 9.889 | 1.00 | 29.46 | O |
| ATOM | 759 | C2 | IPA | I | 1 | −27.705 | −22.576 | 10.659 | 1.00 | 28.43 | C |
| ATOM | 760 | C3 | IPA | I | 1 | −27.051 | −21.536 | 9.761 | 1.00 | 29.38 | C |
| ATOM | 761 | C1 | IPA | I | 1 | −26.658 | −23.290 | 11.505 | 1.00 | 29.05 | C |
| ATOM | 762 | O4 | PEG | J | 1 | −9.547 | −3.756 | 3.308 | 1.00 | 58.36 | O |
| ATOM | 763 | C4 | PEG | J | 1 | −9.883 | −2.657 | 4.163 | 1.00 | 58.25 | C |
| ATOM | 764 | C3 | PEG | J | 1 | −10.404 | −3.182 | 5.495 | 1.00 | 58.20 | C |
| ATOM | 765 | O2 | PEG | J | 1 | −9.394 | −3.967 | 6.123 | 1.00 | 58.21 | O |
| ATOM | 766 | C2 | PEG | J | 1 | −9.294 | −3.694 | 7.520 | 1.00 | 58.10 | C |
| ATOM | 767 | C1 | PEG | J | 1 | −8.668 | −4.896 | 8.219 | 1.00 | 58.14 | C |
| ATOM | 768 | O1 | PEG | J | 1 | −8.328 | −4.553 | 9.567 | 1.00 | 57.92 | O |
| ATOM | 769 | O4 | PEG | K | 1 | −17.405 | −3.800 | 0.184 | 1.00 | 56.32 | O |
| ATOM | 770 | C4 | PEG | K | 1 | −16.334 | −3.212 | −0.563 | 1.00 | 56.15 | C |
| ATOM | 771 | C3 | PEG | K | 1 | −15.142 | −4.162 | −0.561 | 1.00 | 56.11 | C |
| ATOM | 772 | O2 | PEG | K | 1 | −13.969 | −3.470 | −0.980 | 1.00 | 56.06 | O |
| ATOM | 773 | C2 | PEG | K | 1 | −12.916 | −3.565 | −0.022 | 1.00 | 56.02 | C |
| ATOM | 774 | C1 | PEG | K | 1 | −11.625 | −3.968 | −0.725 | 1.00 | 55.96 | C |
| ATOM | 775 | O1 | PEG | K | 1 | −10.774 | −4.666 | 0.192 | 1.00 | 55.92 | O |
| ATOM | 776 | O4 | PEG | L | 1 | −13.711 | −3.323 | −10.947 | 1.00 | 44.91 | O |
| ATOM | 777 | C4 | PEG | L | 1 | −12.606 | −3.777 | −11.736 | 1.00 | 45.15 | C |
| ATOM | 778 | C3 | PEG | L | 1 | −11.386 | −3.950 | −10.840 | 1.00 | 45.15 | C |
| ATOM | 779 | O2 | PEG | L | 1 | −11.705 | −4.840 | −9.772 | 1.00 | 44.93 | O |
| ATOM | 780 | C2 | PEG | L | 1 | −10.541 | −5.478 | −9.251 | 1.00 | 44.99 | C |
| ATOM | 781 | C1 | PEG | L | 1 | −10.722 | −5.748 | −7.762 | 1.00 | 44.88 | C |
| ATOM | 782 | O1 | PEG | L | 1 | −9.798 | −4.947 | −7.014 | 1.00 | 44.90 | O |
| ATOM | 783 | O5 | XYY | H | 1 | −2.944 | −23.249 | 17.367 | 1.00 | 39.93 | O |
| ATOM | 784 | C5 | XYY | H | 1 | −3.890 | −22.210 | 17.084 | 1.00 | 40.74 | C |
| ATOM | 785 | C4 | XYY | H | 1 | −3.202 | −20.848 | 17.066 | 1.00 | 40.86 | C |
| ATOM | 786 | O4 | XYY | H | 1 | −2.367 | −20.771 | 15.904 | 1.00 | 41.03 | O |
| ATOM | 787 | C3 | XYY | H | 1 | −4.236 | −19.708 | 17.096 | 1.00 | 41.18 | C |
| ATOM | 788 | O3 | XYY | H | 1 | −4.987 | −19.677 | 15.872 | 1.00 | 42.08 | O |
| ATOM | 789 | C2 | XYY | H | 1 | −3.667 | −18.313 | 17.398 | 1.00 | 41.08 | C |
| ATOM | 790 | O2 | XYY | H | 1 | −2.852 | −18.349 | 18.581 | 1.00 | 41.52 | O |
| ATOM | 791 | C1 | XYY | H | 1 | −2.876 | −17.695 | 16.241 | 1.00 | 40.98 | C |
| ATOM | 792 | O1 | XYY | H | 1 | −3.615 | −16.613 | 15.659 | 1.00 | 40.51 | O |

TABLE 3
M2TM' G34 coordinates

| HEADER | | | | | | | | |
|---|---|---|---|---|---|---|---|---|
| COMPND | — | #NAME? | | | | | | |
| REMARK | 3 | | | | | | | |
| REMARK | 3 | REFINEMENT. | | | | | | |
| REMARK | 3 | PROGRAM: REFMAC 5.2.0019 | | | | | | |
| REMARK | 3 | AUTHORS: MURSHUDOV, VAGIN, DODSON | | | | | | |
| REMARK | 3 | | | | | | | |
| REMARK | 3 | REFINEMENT TARGET: MAXIMUM LIKELIHOOD | | | | | | |
| REMARK | 3 | | | | | | | |
| REMARK | 3 | DATA USED IN REFINEMENT. | | | | | | |
| REMARK | 3 | RESOLUTION RANGE HIGH (ANGSTROMS): 2.50 | | | | | | |
| REMARK | 3 | RESOLUTION RANGE LOW (ANGSTROMS): 41.31 | | | | | | |
| REMARK | 3 | DATA CUTOFF (SIGMA(F)) : NONE | | | | | | |
| REMARK | 3 | COMPLETENESS FOR RANGE (%) : 65.23 | | | | | | |
| REMARK | 3 | NUMBER OF REFLECTIONS: 2130 | | | | | | |
| REMARK | 3 | | | | | | | |
| REMARK | 3 | FIT TO DATA USED IN REFINEMENT. | | | | | | |
| REMARK | 3 | CROSS-VALIDATION METHOD: THROUGHOUT | | | | | | |
| REMARK | 3 | FREE R VALUE TEST SET SELECTION: RANDOM | | | | | | |
| REMARK | 3 | R VALUE (WORKING + TEST SET): 0.30026 | | | | | | |
| REMARK | 3 | R VALUE (WORKING SET): 0.29874 | | | | | | |
| REMARK | 3 | FREE R VALUE: 0.33337 | | | | | | |
| REMARK | 3 | FREE R VALUE TEST SET SIZE (%): 4.3 | | | | | | |
| REMARK | 3 | FREE R VALUE TEST SET COUNT : 95 | | | | | | |
| REMARK | 3 | | | | | | | |
| REMARK | 3 | FIT IN THE HIGHEST RESOLUTION BIN. | | | | | | |
| REMARK | 3 | TOTAL NUMBER OF BINS USED: 20 | | | | | | |
| REMARK | 3 | BIN RESOLUTION RANGE HIGH: 2.500 | | | | | | |
| REMARK | 3 | BIN RESOLUTION RANGE LOW: 2.565 | | | | | | |
| REMARK | 3 | REFLECTION IN BIN (WORKING SET): 177 | | | | | | |
| REMARK | 3 | BIN COMPLETENESS (WORKING + TEST) (%): 71.81 | | | | | | |
| REMARK | 3 | BIN R VALUE (WORKING SET): 0.431 | | | | | | |
| REMARK | 3 | BIN FREE R VALUE SET COUNT: 9 | | | | | | |
| REMARK | 3 | BIN FREE R VALUE: 0.218 | | | | | | |
| REMARK | 3 | | | | | | | |
| REMARK | 3 | NUMBER OF NON-HYDROGEN ATOMS USED IN REFINEMENT. | | | | | | |
| REMARK | 3 | ALL ATOMS: 728 | | | | | | |
| REMARK | 3 | | | | | | | |
| REMARK | 3 | B VALUES. | | | | | | |
| REMARK | 3 | FROM WILSON PLOT (A**2): NULL | | | | | | |
| REMARK | 3 | MEAN B VALUE (OVERALL, A**2) : 13.153 | | | | | | |
| REMARK | 3 | OVERALL ANISOTROPIC B VALUE. | | | | | | |
| REMARK | 3 | B11 (A**2): −4.95 | | | | | | |
| REMARK | 3 | B22 (A**2): 10.30 | | | | | | |
| REMARK | 3 | B33 (A**2) : −5.35 | | | | | | |
| REMARK | 3 | B12 (A**2): 0.00 | | | | | | |
| REMARK | 3 | B13 (A**2): 0.00 | | | | | | |
| REMARK | 3 | B23 (A**2): 0.00 | | | | | | |
| REMARK | 3 | | | | | | | |
| REMARK | 3 | ESTIMATED OVERALL COORDINATE ERROR. | | | | | | |
| REMARK | 3 | ESU BASED ON R VALUE (A): 0.511 | | | | | | |
| REMARK | 3 | ESU BASED ON FREE R VALUE (A): 0.570 | | | | | | |
| REMARK | 3 | ESU BASED ON MAXIMUM LIKELIHOOD (A): 0.468 | | | | | | |
| REMARK | 3 | ESU FOR B VALUES BASED ON MAXIMUM LIKELIHOOD (A**2): 23.885 | | | | | | |
| REMARK | 3 | | | | | | | |
| REMARK | 3 | CORRELATION COEFFICIENTS. | | | | | | |
| REMARK | 3 | CORRELATION COEFFICIENT FO-FC: 0.883 | | | | | | |
| REMARK | 3 | CORRELATION COEFFICIENT FO-FC FREE: 0.867 | | | | | | |
| REMARK | 3 | RMS DEVIATIONS FROM IDEAL VALUES COUNT RMS WEIGHT | | | | | | |
| REMARK | 3 | | | | | | | |
| REMARK | 3 | ISOTROPIC THERMAL FACTOR RESTRAINTS. COUNT RMS WEIGHT | | | | | | |
| REMARK | 3 | | | | | | | |
| REMARK | 3 | | | | | | | |
| REMARK | 3 | NCS RESTRAINTS STATISTICS | | | | | | |
| REMARK | 3 | NUMBER OF NCS GROUPS: NULL | | | | | | |
| REMARK | 3 | | | | | | | |
| REMARK | 3 | | | | | | | |
| REMARK | 3 | TLS DETAILS | | | | | | |
| REMARK | 3 | NUMBER OF TLS GROUPS: NULL | | | | | | |
| REMARK | 3 | | | | | | | |
| REMARK | 3 | | | | | | | |
| REMARK | 3 | BULK SOLVENT MODELLING. | | | | | | |
| REMARK | 3 | METHOD USED: MASK | | | | | | |
| REMARK | 3 | PARAMETERS FOR MASK CALCULATION | | | | | | |
| REMARK | 3 | VDW PROBE RADIUS: 1.20 | | | | | | |
| REMARK | 3 | ION PROBE RADIUS: 0.80 | | | | | | |
| REMARK | 3 | SHRINKAGE RADIUS: 0.80 | | | | | | |
| REMARK | 3 | | | | | | | |
| REMARK | 3 | OTHER REFINEMENT REMARKS: | | | | | | |
| REMARK | 3 | HYDROGENS HAVE BEEN ADDED IN THE RIDING POSITIONS | | | | | | |
| REMARK | 3 | | | | | | | |
| CRYST1 | 4 | 8.740 | 77.860 | 48.610 | 90.00 | 90.00 | 90.00 | C 2 2 21 |

| | | | | |
|---|---|---|---|---|
| SCALE1 | 0.020517 | 0.000000 | 0.000000 | 0.00000 |
| SCALE2 | 0.000000 | 0.012844 | 0.000000 | 0.00000 |
| SCALE3 | 0.000000 | 0.000000 | 0.020572 | 0.00000 |

| | | | | | | | | | | | |
|---|---|---|---|---|---|---|---|---|---|---|---|
| ATOM | | 1 | BR4 | BRB C | 1 | −7.246 | −0.397 | −4.688 | 1.00 | 34.10 | BR |
| ATOM | | 2 | C4 | BRB C | 1 | −6.207 | 1.123 | −5.107 | 1.00 | 29.88 | C |
| ATOM | | 3 | C3 | BRB C | 1 | −5.996 | 1.453 | −6.443 | 1.00 | 30.24 | C |
| ATOM | | 4 | C2 | BRB C | 1 | −5.236 | 2.574 | −6.761 | 1.00 | 30.20 | C |
| ATOM | | 5 | C5 | BRB C | 1 | −5.666 | 1.898 | −4.083 | 1.00 | 30.06 | C |
| ATOM | | 6 | C6 | BRB C | 1 | −4.901 | 3.015 | −4.402 | 1.00 | 29.86 | C |
| ATOM | | 7 | C1 | BRB C | 1 | −4.686 | 3.348 | −5.740 | 1.00 | 29.45 | C |
| ATOM | | 8 | C7 | BRB C | 1 | −3.854 | 4.559 | −6.089 | 1.00 | 25.72 | C |
| ATOM | | 9 | O1 | BRB C | 1 | −4.124 | 5.561 | −5.099 | 1.00 | 24.48 | O |
| ATOM | 1 | 0 | N | PRO C | 25 | −3.077 | 4.788 | −7.070 | 1.00 | 21.30 | N |
| ATOM | 1 | 1 | CA | PRO C | 25 | −2.291 | 6.015 | −7.198 | 1.00 | 19.89 | C |
| ATOM | 1 | 2 | CB | PRO C | 25 | −1.477 | 5.773 | −8.467 | 1.00 | 20.31 | C |
| ATOM | 1 | 3 | CG | PRO C | 25 | −1.263 | 4.303 | −8.470 | 1.00 | 20.73 | C |
| ATOM | 1 | 4 | CD | PRO C | 25 | −2.538 | 3.714 | −7.930 | 1.00 | 21.07 | C |
| ATOM | 1 | 5 | C | PRO C | 25 | −3.141 | 7.280 | −7.342 | 1.00 | 18.89 | C |
| ATOM | 1 | 6 | O | PRO C | 25 | −2.714 | 8.350 | −6.915 | 1.00 | 18.57 | O |
| ATOM | 1 | 7 | N | LEU C | 26 | −4.330 | 7.153 | −7.930 | 1.00 | 17.34 | N |
| ATOM | 1 | 8 | CA | LEU C | 26 | −5.244 | 8.294 | −8.046 | 1.00 | 16.23 | C |
| ATOM | 1 | 9 | CB | LEU C | 26 | −6.488 | 7.924 | −8.868 | 1.00 | 16.45 | C |
| ATOM | 2 | 0 | CG | LEU C | 26 | −7.640 | 8.939 | −8.918 | 1.00 | 16.44 | C |
| ATOM | 2 | 1 | CD1 | LEU C | 26 | −8.869 | 8.354 | −9.609 | 1.00 | 16.56 | C |
| ATOM | 2 | 2 | CD2 | LEU C | 26 | −7.220 | 10.257 | −9.575 | 1.00 | 15.87 | C |
| ATOM | 2 | 3 | C | LEU C | 26 | −5.642 | 8.827 | −6.665 | 1.00 | 15.48 | C |
| ATOM | 2 | 4 | O | LEU C | 26 | −5.705 | 10.044 | −6.454 | 1.00 | 14.83 | O |
| ATOM | 2 | 5 | N | VAL C | 27 | −5.907 | 7.915 | −5.736 | 1.00 | 14.66 | N |
| ATOM | 2 | 6 | CA | VAL C | 27 | −6.300 | 8.296 | −4.378 | 1.00 | 14.42 | C |
| ATOM | 2 | 7 | CB | VAL C | 27 | −6.872 | 7.092 | −3.595 | 1.00 | 14.64 | C |
| ATOM | 2 | 8 | CG1 | VAL C | 27 | −7.226 | 7.493 | −2.167 | 1.00 | 15.16 | C |
| ATOM | 2 | 9 | CG2 | VAL C | 27 | −8.104 | 6.544 | −4.305 | 1.00 | 14.88 | C |
| ATOM | 3 | 0 | C | VAL C | 27 | −5.117 | 8.939 | −3.642 | 1.00 | 14.15 | C |
| ATOM | 3 | 1 | O | VAL C | 27 | −5.291 | 9.925 | −2.923 | 1.00 | 13.94 | O |
| ATOM | 3 | 2 | N | VAL C | 28 | −3.917 | 8.397 | −3.849 | 1.00 | 13.68 | N |
| ATOM | 3 | 3 | CA | VAL C | 28 | −2.699 | 8.987 | −3.284 | 1.00 | 13.17 | C |
| ATOM | 3 | 4 | CB | VAL C | 28 | −1.460 | 8.090 | −3.545 | 1.00 | 13.49 | C |
| ATOM | 3 | 5 | CG1 | VAL C | 28 | −0.162 | 8.807 | −3.148 | 1.00 | 14.36 | C |
| ATOM | 3 | 6 | CG2 | VAL C | 28 | −1.608 | 6.771 | −2.793 | 1.00 | 13.97 | C |
| ATOM | 3 | 7 | C | VAL C | 28 | −2.490 | 10.401 | −3.829 | 1.00 | 12.24 | C |
| ATOM | 3 | 8 | O | VAL C | 28 | −2.292 | 11.345 | −3.061 | 1.00 | 12.05 | O |
| ATOM | 3 | 9 | N | ALA C | 29 | −2.562 | 10.548 | −5.152 | 1.00 | 11.58 | N |
| ATOM | 4 | 0 | CA | ALA C | 29 | −2.430 | 11.858 | −5.786 | 1.00 | 10.97 | C |
| ATOM | 4 | 1 | CB | ALA C | 29 | −2.2571 | 11.734 | −7.303 | 1.00 | 11.39 | C |
| ATOM | 4 | 2 | C | ALA C | 29 | −3.443 | 12.865 | −5.226 | 1.00 | 10.57 | C |
| ATOM | 4 | 3 | O | ALA C | 29 | −3.077 | 13.984 | −4.849 | 1.00 | 10.12 | O |
| ATOM | 4 | 4 | N | ALA C | 30 | −4.710 | 12.455 | −5.157 | 1.00 | 10.37 | N |
| ATOM | 4 | 5 | CA | ALA C | 30 | −5.776 | 13.311 | −4.629 | 1.00 | 10.21 | C |
| ATOM | 4 | 6 | CB | ALA C | 30 | −7.124 | 12.632 | −4.780 | 1.00 | 10.68 | C |
| ATOM | 4 | 7 | C | ALA C | 30 | −5.541 | 13.692 | −3.168 | 1.00 | 10.09 | C |
| ATOM | 4 | 8 | O | ALA C | 30 | −5.815 | 14.825 | −2.768 | 1.00 | 10.36 | O |
| ATOM | 4 | 9 | N | SER C | 31 | −5.040 | 12.736 | −2.379 | 1.00 | 9.78 | N |
| ATOM | 5 | 0 | CA | SER C | 31 | −4.720 | 12.976 | −0.969 | 1.00 | 9.35 | C |
| ATOM | 5 | 1 | CB | SER C | 31 | −4.306 | 11.678 | −0.276 | 1.00 | 9.78 | C |
| ATOM | 5 | 2 | OG | SER C | 31 | −5.418 | 10.805 | −0.159 | 1.00 | 10.25 | O |
| ATOM | 5 | 3 | C | SER C | 31 | −3.636 | 14.041 | −0.810 | 1.00 | 9.12 | C |
| ATOM | 5 | 4 | O | SER C | 31 | −3.758 | 14.955 | 0.010 | 1.00 | 8.45 | O |
| ATOM | 5 | 5 | N | ILE C | 32 | −2.580 | 13.924 | −1.611 | 1.00 | 8.60 | N |
| ATOM | 5 | 6 | CA | ILE C | 32 | −1.522 | 14.928 | −1.622 | 1.00 | 8.94 | C |
| ATOM | 5 | 7 | CB | ILE C | 32 | −0.374 | 14.507 | −2.569 | 1.00 | 8.81 | C |
| ATOM | 5 | 8 | CG1 | ILE C | 32 | 0.332 | 13.262 | −2.013 | 1.00 | 9.00 | C |
| ATOM | 5 | 9 | CD1 | ILE C | 32 | 1.219 | 12.574 | −3.026 | 1.00 | 9.52 | C |
| ATOM | 6 | 0 | CG2 | ILE C | 32 | 0.627 | 15.649 | −2.764 | 1.00 | 9.97 | C |
| ATOM | 6 | 1 | C | ILE C | 32 | −2.087 | 16.289 | −2.030 | 1.00 | 9.14 | C |
| ATOM | 6 | 2 | O | ILE C | 32 | −1.809 | 17.306 | −1.393 | 1.00 | 9.10 | O |
| ATOM | 6 | 3 | N | ILE C | 33 | −2.895 | 16.286 | −3.087 | 1.00 | 9.46 | N |
| ATOM | 6 | 4 | CA | ILE C | 33 | −3.479 | 17.516 | −3.616 | 1.00 | 10.25 | C |
| ATOM | 6 | 5 | CB | ILE C | 33 | −4.134 | 17.268 | −5.003 | 1.00 | 10.49 | C |
| ATOM | 6 | 6 | CG1 | ILE C | 33 | −3.030 | 17.252 | −6.068 | 1.00 | 11.66 | C |
| ATOM | 6 | 7 | CD1 | ILE C | 33 | −3.438 | 16.717 | −7.429 | 1.00 | 14.10 | C |
| ATOM | 6 | 8 | CG2 | ILE C | 33 | −5.211 | 18.317 | −5.325 | 1.00 | 10.84 | C |
| ATOM | 6 | 9 | C | ILE C | 33 | −4.410 | 18.199 | −2.603 | 1.00 | 10.48 | C |
| ATOM | 7 | 0 | O | ILE C | 33 | −4.390 | 19.422 | −2.478 | 1.00 | 10.41 | O |
| ATOM | 7 | 1 | N | GLY C | 34 | −5.188 | 17.409 | −1.865 | 1.00 | 10.62 | N |
| ATOM | 7 | 2 | CA | GLY C | 34 | −6.090 | 17.951 | −0.829 | 1.00 | 11.16 | C |
| ATOM | 7 | 3 | C | GLY C | 34 | −5.327 | 18.693 | 0.278 | 1.00 | 11.21 | C |
| ATOM | 7 | 4 | O | GLY C | 34 | −5.753 | 19.764 | 0.765 | 1.00 | 12.07 | O |
| ATOM | 7 | 5 | N | ILE C | 35 | −4.177 | 18.147 | 0.650 | 1.00 | 10.59 | N |
| ATOM | 7 | 6 | CA | ILE C | 35 | −3.338 | 18.784 | 1.653 | 1.00 | 10.26 | C |
| ATOM | 7 | 7 | CB | ILE C | 35 | −2.272 | 17.806 | 2.226 | 1.00 | 10.31 | C |
| ATOM | 7 | 8 | CG1 | ILE C | 35 | −2.964 | 16.588 | 2.868 | 1.00 | 10.82 | C |
| ATOM | 7 | 9 | CD1 | ILE C | 35 | −2.041 | 15.394 | 3.159 | 1.00 | 10.95 | C |
| ATOM | 8 | 0 | CG2 | ILE C | 35 | −1.393 | 18.526 | 3.242 | 1.00 | 10.23 | C` |
| ATOM | 8 | 1 | C | ILE C | 35 | −2.722 | 20.067 | 1.077 | 1.00 | 9.81 | C |
| ATOM | 8 | 2 | O | ILE C | 35 | −2.755 | 21.119 | 1.722 | 1.00 | 10.29 | O |
| ATOM | 8 | 3 | N | LEU C | 36 | −2.206 | 19.979 | −0.151 | 1.00 | 9.58 | N |
| ATOM | 8 | 4 | CA | LEU C | 36 | −1.612 | 21.124 | −0.827 | 1.00 | 9.06 | C |
| ATOM | 8 | 5 | CB | LEU C | 36 | −1.048 | 20.717 | −2.194 | 1.00 | 8.93 | C |
| ATOM | 8 | 6 | CG | LEU C | 36 | −0.481 | 21.863 | −3.039 | 1.00 | 9.66 | C |
| ATOM | 8 | 7 | CD1 | LEU C | 36 | 0.693 | 22.543 | −2.333 | 1.00 | 10.84 | C |
| ATOM | 8 | 8 | CD2 | LEU C | 36 | −0.081 | 21.392 | −4.437 | 1.00 | 9.68 | C |
| ATOM | 8 | 9 | C | LEU C | 36 | −2.634 | 22.246 | −0.988 | 1.00 | 8.68 | C |
| ATOM | 9 | 0 | O | LEU C | 36 | −2.335 | 23.409 | −0.698 | 1.00 | 8.71 | O |
| ATOM | 9 | 1 | N | HIS C | 37 | −3.835 | 21.883 | −1.432 | 1.00 | 8.48 | N |
| ATOM | 9 | 2 | CA | HIS C | 37 | −4.913 | 22.851 | −1.629 | 1.00 | 8.49 | C |
| ATOM | 9 | 3 | CB | HIS C | 37 | −6.176 | 22.144 | −2.129 | 1.00 | 8.57 | C |
| ATOM | 9 | 4 | CG | HIS C | 37 | −7.212 | 23.067 | −2.692 | 1.00 | 9.52 | C |
| ATOM | 9 | 5 | ND1 | HIS C | 37 | −8.298 | 22.606 | −3.402 | 1.00 | 9.81 | N |
| ATOM | 9 | 6 | CE1 | HIS C | 37 | −9.045 | 23.629 | −3.775 | 1.00 | 10.74 | C |
| ATOM | 9 | 7 | NE2 | HIS C | 37 | −8.482 | 24.739 | −3.336 | 1.00 | 10.44 | N |
| ATOM | 9 | 8 | CD2 | HIS C | 37 | −7.329 | 24.416 | −2.658 | 1.00 | 9.95 | C |
| ATOM | 9 | 9 | C | HIS C | 37 | −5.218 | 23.632 | −0.342 | 1.00 | 8.53 | C |
| ATOM | 10 | 0 | O | HIS C | 37 | −5.362 | 24.858 | −0.380 | 1.00 | 8.76 | O |
| ATOM | 10 | 1 | N | LEU C | 38 | −5.299 | 22.938 | 0.797 | 1.00 | 8.37 | N |
| ATOM | 10 | 2 | CA | LEU C | 38 | −5.590 | 23.637 | 2.052 | 1.00 | 9.13 | C |
| ATOM | 10 | 3 | CB | LEU C | 38 | −5.838 | 22.674 | 3.220 | 1.00 | 8.96 | C |
| ATOM | 10 | 4 | CG | LEU C | 38 | −6.107 | 23.401 | 4.552 | 1.00 | 10.16 | C |
| ATOM | 10 | 5 | CD1 | LEU C | 38 | −7.349 | 24.300 | 4.476 | 1.00 | 12.08 | C |
| ATOM | 10 | 6 | CD2 | LEU C | 38 | −6.230 | 22.432 | 5.710 | 1.00 | 10.86 | C |
| ATOM | 10 | 7 | C | LEU C | 38 | −4.470 | 24.603 | 2.397 | 1.00 | 8.70 | C |
| ATOM | 10 | 8 | O | LEU C | 38 | −4.727 | 25.743 | 2.780 | 1.00 | 8.47 | O |
| ATOM | 10 | 9 | N | ILE C | 39 | −3.229 | 24.148 | 2.246 | 1.00 | 8.68 | N |
| ATOM | 11 | 0 | CA | ILE C | 39 | −2.076 | 24.996 | 2.535 | 1.00 | 8.97 | C |
| ATOM | 11 | 1 | CB | ILE C | 39 | −0.751 | 24.233 | 2.327 | 1.00 | 9.08 | C |
| ATOM | 11 | 2 | CG1 | ILE C | 39 | −0.629 | 23.111 | 3.365 | 1.00 | 9.46 | C |
| ATOM | 11 | 3 | CD1 | ILE C | 39 | 0.579 | 22.210 | 3.166 | 1.00 | 10.99 | C |
| ATOM | 11 | 4 | CG2 | ILE C | 39 | 0.445 | 25.190 | 2.411 | 1.00 | 1.05 | C |
| ATOM | 11 | 5 | C | ILE C | 39 | −2.112 | 26.254 | 1.670 | 1.00 | 8.90 | C |
| ATOM | 11 | 6 | O | ILE C | 39 | −1.994 | 27.373 | 2.178 | 1.00 | 8.90 | O |
| ATOM | 11 | 7 | N | LEU C | 40 | −2.306 | 26.066 | 0.366 | 1.00 | 9.09 | N |
| ATOM | 11 | 8 | CA | LEU C | 40 | −2.348 | 27.187 | −0.570 | 1.00 | 9.27 | C |
| ATOM | 11 | 9 | CB | LEU C | 40 | −2.515 | 26.679 | −2.003 | 1.00 | 9.10 | C |
| ATOM | 12 | 0 | CG | LEU C | 40 | −1.317 | 25.962 | −2.621 | 1.00 | 9.09 | C |
| ATOM | 12 | 1 | CD1 | LEU C | 40 | −1.695 | 25.414 | −3.997 | 1.00 | 9.42 | C |
| ATOM | 12 | 2 | CD2 | LEU C | 40 | −0.089 | 26.868 | −2.719 | 1.00 | 10.51 | C |
| ATOM | 12 | 3 | C | LEU C | 40 | −3.472 | 28.166 | −0.242 | 1.00 | 9.74 | C |
| ATOM | 12 | 4 | O | LEU C | 40 | −3.270 | 29.385 | −0.271 | 1.00 | 9.52 | O |
| ATOM | 12 | 5 | N | TRP C | 41 | −4.648 | 27.625 | 0.069 | 1.00 | 10.27 | N |
| ATOM | 12 | 6 | CA | TRP C | 41 | −5.815 | 28.450 | 0.372 | 1.00 | 11.06 | C |
| ATOM | 12 | 7 | CB | TRP C | 41 | −7.086 | 27.598 | 0.456 | 1.00 | 11.42 | C |
| ATOM | 12 | 8 | CG | TRP C | 41 | −8.323 | 28.404 | 0.723 | 1.00 | 11.56 | C |
| ATOM | 12 | 9 | CD1 | TRP C | 41 | −9.068 | 29.099 | −0.192 | 1.00 | 12.48 | C |
| ATOM | 13 | 0 | NE1 | TRP C | 41 | −10.132 | 29.714 | 0.432 | 1.00 | 12.61 | N |
| ATOM | 13 | 1 | CE2 | TRP C | 41 | −10.085 | 29.425 | 1.774 | 1.00 | 11.74 | C |
| ATOM | 13 | 2 | CD2 | TRP C | 41 | −8.958 | 28.599 | 1.991 | 1.00 | 11.25 | C |
| ATOM | 13 | 3 | CE3 | TRP C | 41 | −8.682 | 28.161 | 3.292 | 1.00 | 13.02 | C |
| ATOM | 13 | 4 | CZ3 | TRP C | 41 | −9.528 | 28.554 | 4.326 | 1.00 | 12.28 | C |
| ATOM | 13 | 5 | CH2 | TRP C | 41 | −10.644 | 29.372 | 4.077 | 1.00 | 12.40 | C |
| ATOM | 13 | 6 | CZ2 | TRP C | 41 | −10.939 | 29.819 | 2.813 | 1.00 | 11.25 | C |
| ATOM | 13 | 7 | C | TRP C | 41 | −5.620 | 29.274 | 1.651 | 1.00 | 11.73 | C |
| ATOM | 13 | 8 | O | TRP C | 41 | −5.951 | 30.463 | 1.683 | 1.00 | 11.22 | O |
| ATOM | 13 | 9 | N | ILE C | 42 | −5.074 | 28.644 | 2.691 | 1.00 | 12.64 | N |
| ATOM | 14 | 0 | CA | ILE C | 42 | −4.783 | 29.341 | 3.947 | 1.00 | 14.12 | C |
| ATOM | 14 | 1 | CB | ILE C | 42 | −4.219 | 28.381 | 5.026 | 1.00 | 13.94 | C |
| ATOM | 14 | 2 | CG1 | ILE C | 42 | −5.309 | 27.420 | 5.510 | 1.00 | 14.88 | C |
| ATOM | 14 | 3 | CD1 | ILE C | 42 | −4.830 | 26.387 | 6.519 | 1.00 | 14.88 | C |
| ATOM | 14 | 4 | CG2 | ILE C | 42 | −3.624 | 29.167 | 6.203 | 1.00 | 14.75 | C |
| ATOM | 14 | 5 | C | ILE C | 42 | −3.812 | 30.498 | 3.707 | 1.00 | 14.66 | C |
| ATOM | 14 | 6 | O | ILE C | 42 | −4.043 | 31.615 | 4.180 | 1.00 | 14.96 | O |
| ATOM | 14 | 7 | N | LEU C | 43 | −2.740 | 30.228 | 2.963 | 1.00 | 15.33 | N |
| ATOM | 14 | 8 | CA | LEU C | 43 | −1.751 | 31.258 | 2.625 | 1.00 | 16.22 | C |
| ATOM | 14 | 9 | CB | LEU C | 43 | −0.532 | 30.635 | 1.936 | 1.00 | 16.25 | C |
| ATOM | 15 | 0 | CG | LEU C | 43 | 0.329 | 29.731 | 2.829 | 1.00 | 16.68 | C |
| ATOM | 15 | 1 | CD1 | LEU C | 43 | 1.322 | 28.935 | 1.994 | 1.00 | 16.94 | C |
| ATOM | 15 | 2 | CD2 | LEU C | 43 | 1.057 | 30.537 | 3.899 | 1.00 | 17.64 | C |
| ATOM | 15 | 3 | C | LEU C | 43 | −2.341 | 32.386 | 1.778 | 1.00 | 17.06 | C |
| ATOM | 15 | 4 | O | LEU C | 43 | −1.981 | 33.551 | 1.959 | 1.00 | 17.38 | O |
| ATOM | 15 | 5 | N | ASP C | 44 | −3.241 | 32.040 | 0.859 | 1.00 | 17.75 | N |
| ATOM | 15 | 6 | CA | ASP C | 44 | −3.936 | 33.037 | 0.048 | 1.00 | 18.96 | C |
| ATOM | 15 | 7 | CB | ASP C | 44 | −4.802 | 32.369 | −1.025 | 1.00 | 19.20 | C |
| ATOM | 15 | 8 | CG | ASP C | 44 | −5.493 | 33.380 | −1.925 | 1.00 | 19.98 | C |
| ATOM | 15 | 9 | OD1 | ASP C | 44 | −6.739 | 33.436 | −1.913 | 1.00 | 21.74 | O |
| ATOM | 16 | 0 | OD2 | ASP C | 44 | −4.789 | 34.126 | −2.631 | 1.00 | 21.92 | O |
| ATOM | 16 | 1 | C | ASP C | 44 | −4.798 | 33.948 | 0.918 | 1.00 | 19.52 | C |
| ATOM | 16 | 2 | O | ASP C | 44 | −4.799 | 35.167 | 0.730 | 1.00 | 19.66 | O |
| ATOM | 16 | 3 | N | ARG C | 45 | −5.513 | 33.353 | 1.872 | 1.00 | 20.43 | N |
| ATOM | 16 | 4 | CA | ARG C | 45 | −6.428 | 34.090 | 2.749 | 1.00 | 21.53 | C |
| ATOM | 16 | 5 | CB | ARG C | 45 | −7.374 | 33.137 | 3.485 | 1.00 | 21.60 | C |
| ATOM | 16 | 6 | CG | ARG C | 45 | −8.377 | 32.405 | 2.597 | 1.00 | 22.52 | C |
| ATOM | 16 | 7 | CD | ARG C | 45 | −9.491 | 33.317 | 2.081 | 1.00 | 24.33 | C |
| ATOM | 16 | 8 | NE | ARG C | 45 | −9.165 | 33.922 | 0.794 | 1.00 | 25.25 | N |
| ATOM | 16 | 9 | CZ | ARG C | 45 | −9.766 | 34.956 | 0.453 | 1.00 | 30.47 | C |
| ATOM | 17 | 0 | NH1 | ARG C | 45 | −10.645 | 35.621 | 1.196 | 1.00 | 31.17 | N |
| ATOM | 17 | 1 | NH2 | ARG C | 45 | −9.448 | 35.411 | −0.753 | 1.00 | 30.73 | N |
| ATOM | 17 | 2 | C | ARG C | 45 | −5.693 | 34.973 | 3.753 | 1.00 | 22.35 | C |
| ATOM | 17 | 3 | O | ARG C | 45 | −6.139 | 36.082 | 4.050 | 1.00 | 22.50 | O |
| ATOM | 17 | 4 | N | LEU C | 46 | −4.574 | 34.474 | 4.277 | 1.00 | 23.25 | N |
| ATOM | 17 | 5 | CA | LEU C | 46 | −3.730 | 35.244 | 5.193 | 1.00 | 24.08 | C |
| ATOM | 17 | 6 | CB | LEU C | 46 | −2.693 | 34.348 | 5.873 | 1.00 | 24.31 | C |
| ATOM | 17 | 7 | CG | LEU C | 46 | −3.149 | 33.207 | 6.790 | 1.00 | 24.74 | C |
| ATOM | 17 | 8 | CD1 | LEU C | 46 | −1.950 | 32.364 | 7.200 | 1.00 | 25.15 | C |
| ATOM | 17 | 9 | CD2 | LEU C | 46 | −3.899 | 33.712 | 8.017 | 1.00 | 25.49 | C |
| ATOM | 18 | 0 | C | LEU C | 46 | −3.033 | 36.398 | 4.476 | 1.00 | 24.60 | C |
| ATOM | 18 | 1 | O | LEU C | 46 | −2.662 | 36.292 | 3.305 | 1.00 | 24.95 | O |
| ATOM | 18 | 2 | N | NH2 C | 47 | −2.452 | 37.440 | 4.881 | 1.00 | 25.13 | N |
| ATOM | 18 | 3 | BR4 | BRB D | 1 | −14.241 | −0.631 | −1.738 | 1.00 | 35.49 | BR |
| ATOM | 18 | 4 | C4 | BRB D | 1 | −14.654 | 0.711 | −2.991 | 1.00 | 30.58 | C |
| ATOM | 18 | 5 | C3 | BRB D | 1 | −15.962 | 1.182 | −3.081 | 1.00 | 31.06 | C |
| ATOM | 18 | 6 | C2 | BRB D | 1 | −16.267 | 2.172 | −4.006 | 1.00 | 31.02 | C |
| ATOM | 18 | 7 | C5 | BRB D | 1 | −13.646 | 1.206 | −3.813 | 1.00 | 31.21 | C |
| ATOM | 18 | 8 | C6 | BRB D | 1 | −13.955 | 2.196 | −4.740 | 1.00 | 31.01 | C |
| ATOM | 18 | 9 | C1 | BRB D | 1 | −15.261 | 2.671 | −4.834 | 1.00 | 29.86 | C |
| ATOM | 19 | 0 | C7 | BRB D | 1 | −15.583 | 3.742 | −5.846 | 1.00 | 25.19 | C |
| ATOM | 19 | 1 | O1 | BRB D | 1 | −14.621 | 4.792 | −5.694 | 1.00 | 23.98 | O |
| ATOM | 19 | 2 | N | PRO D | 25 | −16.596 | 3.795 | −6.604 | 1.00 | 19.89 | N |
| ATOM | 19 | 3 | CA | PRO D | 25 | −16.866 | 4.901 | −7.519 | 1.00 | 18.19 | C |
| ATOM | 19 | 4 | CB | PRO D | 25 | −18.236 | 4.539 | −8.086 | 1.00 | 18.55 | C |
| ATOM | 19 | 5 | CG | PRO D | 25 | −18.213 | 3.035 | −8.139 | 1.00 | 19.04 | C |
| ATOM | 19 | 6 | CD | PRO D | 25 | −17.281 | 2.561 | −7.037 | 1.00 | 19.33 | C |
| ATOM | 19 | 7 | C | PRO D | 25 | −16.908 | 6.271 | −6.833 | 1.00 | 17.35 | C |
| ATOM | 19 | 8 | O | PRO D | 25 | −16.433 | 7.255 | −7.409 | 1.00 | 16.64 | O |
| ATOM | 19 | 9 | N | LEU D | 26 | −17.450 | 6.328 | −5.616 | 1.00 | 16.14 | N |
| ATOM | 20 | 0 | CA | LEU D | 26 | −17.514 | 7.574 | −4.851 | 1.00 | 15.06 | C |
| ATOM | 20 | 1 | CB | LEU D | 26 | −18.256 | 7.366 | −3.525 | 1.00 | 14.98 | C |
| ATOM | 20 | 2 | CG | LEU D | 26 | −18.309 | 8.563 | −2.562 | 1.00 | 15.38 | C |
| ATOM | 20 | 3 | CD1 | LEU D | 26 | −18.995 | 9.771 | −3.202 | 1.00 | 15.04 | C |
| ATOM | 20 | 4 | CD2 | LEU D | 26 | −18.986 | 8.175 | −1.252 | 1.00 | 14.85 | C |
| ATOM | 20 | 5 | C | LEU D | 26 | −16.128 | 8.168 | −4.585 | 1.00 | 14.34 | C |
| ATOM | 20 | 6 | O | LEU D | 26 | −15.923 | 9.371 | −4.747 | 1.00 | 13.56 | O |
| ATOM | 20 | 7 | N | VAL D | 27 | −15.194 | 7.322 | −4.164 | 1.00 | 13.97 | N |
| ATOM | 20 | 8 | CA | VAL D | 27 | −13.845 | 7.772 | −3.822 | 1.00 | 13.78 | C |
| ATOM | 20 | 9 | CB | VAL D | 27 | −13.080 | 6.714 | −2.988 | 1.00 | 13.93 | C |
| ATOM | 21 | 0 | CG1 | VAL D | 27 | −11.714 | 7.239 | −2.558 | 1.00 | 14.32 | C |
| ATOM | 21 | 1 | CG2 | VAL D | 27 | −13.887 | 6.342 | −1.761 | 1.00 | 14.46 | C |
| ATOM | 21 | 2 | C | VAL D | 27 | −13.071 | 8.157 | −5.085 | 1.00 | 13.48 | C |
| ATOM | 21 | 3 | O | VAL D | 27 | −12.336 | 9.144 | −5.086 | 1.00 | 13.26 | O |
| ATOM | 21 | 4 | N | VAL D | 28 | −13.255 | 7.385 | −6.153 | 1.00 | 12.91 | N |
| ATOM | 21 | 5 | CA | VAL D | 28 | −12.673 | 7.716 | −7.456 | 1.00 | 12.91 | C |
| ATOM | 21 | 6 | CB | VAL D | 28 | −12.966 | 6.616 | −8.510 | 1.00 | 13.19 | C |
| ATOM | 21 | 7 | CG1 | VAL D | 28 | −12.497 | 7.043 | −9.899 | 1.00 | 12.97 | C |
| ATOM | 21 | 8 | CG2 | VAL D | 28 | −12.298 | 5.314 | −8.111 | 1.00 | 13.54 | C |
| ATOM | 21 | 9 | C | VAL D | 28 | −13.179 | 9.080 | −7.944 | 1.00 | 12.32 | C |
| ATOM | 22 | 0 | O | VAL D | 28 | −12.380 | 9.952 | −8.306 | 1.00 | 12.40 | O |
| ATOM | 22 | 1 | N | ALA D | 29 | −14.502 | 9.254 | −7.951 | 1.00 | 11.67 | N |
| ATOM | 22 | 2 | CA | ALA D | 29 | −15.112 | 10.527 | −8.340 | 1.00 | 10.93 | C |
| ATOM | 22 | 3 | CB | ALA D | 29 | −16.631 | 10.435 | −8.269 | 1.00 | 11.38 | C |
| ATOM | 22 | 4 | C | ALA D | 29 | −14.596 | 11.696 | −7.497 | 1.00 | 10.78 | C |
| ATOM | 22 | 5 | O | ALA D | 29 | −14.224 | 12.740 | −8.044 | 1.00 | 10.31 | O |
| ATOM | 22 | 6 | N | ALA D | 30 | −14.554 | 11.514 | −6.177 | 1.00 | 10.30 | N |
| ATOM | 22 | 7 | CA | ALA D | 30 | −14.064 | 12.564 | −5.273 | 1.00 | 10.21 | C |
| ATOM | 22 | 8 | CB | ALA D | 30 | −14.271 | 12.160 | −3.823 | 1.00 | 10.50 | C |
| ATOM | 22 | 9 | C | ALA D | 30 | −12.602 | 12.921 | −5.516 | 1.00 | 9.96 | C |
| ATOM | 23 | 0 | O | ALA D | 30 | −12.220 | 14.092 | −5.444 | 1.00 | 9.93 | O |
| ATOM | 23 | 1 | N | SER D | 31 | −11.793 | 11.906 | −5.802 | 1.00 | 9.80 | N |
| ATOM | 23 | 2 | CA | SER D | 31 | −10.378 | 12.093 | −6.099 | 1.00 | 9.89 | C |
| ATOM | 23 | 3 | CB | SER D | 31 | −9.690 | 10.739 | −6.273 | 1.00 | 10.05 | C |
| ATOM | 23 | 4 | OG | SER D | 31 | −9.587 | 10.080 | −5.025 | 1.00 | 10.93 | O |
| ATOM | 23 | 5 | C | SER D | 31 | −10.191 | 12.964 | −7.338 | 1.00 | 9.62 | C |
| ATOM | 23 | 6 | O | SER D | 31 | −9.405 | 13.918 | −7.325 | 1.00 | 9.46 | O |
| ATOM | 23 | 7 | N | ILE D | 32 | −10.937 | 12.638 | −8.396 | 1.00 | 9.51 | N |
| ATOM | 23 | 8 | CA | ILE D | 32 | −10.921 | 13.406 | −9.641 | 1.00 | 9.92 | C |
| ATOM | 23 | 9 | CB | ILE D | 32 | −11.806 | 12.721 | −10.720 | 1.00 | 10.01 | C |
| ATOM | 24 | 0 | CG1 | ILE D | 32 | −11.192 | 11.374 | −11.114 | 1.00 | 10.66 | C |
| ATOM | 24 | 1 | CD1 | ILE D | 32 | −12.173 | 10.413 | −11.794 | 1.00 | 12.86 | C |
| ATOM | 24 | 2 | CG2 | ILE D | 32 | −11.992 | 13.623 | −11.949 | 1.00 | 10.29 | C |
| ATOM | 24 | 3 | C | ILE D | 32 | −11.390 | 14.836 | −9.374 | 1.00 | 9.87 | C |
| ATOM | 24 | 4 | O | ILE D | 32 | −10.765 | 15.801 | −9.817 | 1.00 | 9.73 | O |
| ATOM | 24 | 5 | N | ILE D | 33 | −12.474 | 14.962 | −8.614 | 1.00 | 9.92 | N |
| ATOM | 24 | 6 | CA | ILE D | 33 | −13.053 | 16.271 | −8.315 | 1.00 | 10.49 | C |
| ATOM | 24 | 7 | CB | ILE D | 33 | −14.465 | 16.126 | −7.695 | 1.00 | 10.89 | C |
| ATOM | 24 | 8 | CG1 | ILE D | 33 | −15.457 | 15.776 | −8.812 | 1.00 | 11.58 | C |
| ATOM | 24 | 9 | CD1 | ILE D | 33 | −16.782 | 15.208 | −8.354 | 1.00 | 13.67 | C |
| ATOM | 25 | 0 | CG2 | ILE D | 33 | −14.874 | 17.394 | −6.955 | 1.00 | 11.08 | C |
| ATOM | 25 | 1 | C | ILE D | 33 | −12.099 | 17.138 | −7.479 | 1.00 | 10.45 | C |
| ATOM | 25 | 2 | O | ILE D | 33 | −11.991 | 18.346 | −7.713 | 1.00 | 9.92 | O |
| ATOM | 25 | 3 | N | GLY D | 34 | −11.394 | 16.517 | −6.536 | 1.00 | 10.56 | N |
| ATOM | 25 | 4 | CA | GLY D | 34 | −10.384 | 17.218 | −5.734 | 1.00 | 10.86 | C |
| ATOM | 25 | 5 | C | GLY D | 34 | −9.270 | 17.813 | −6.608 | 1.00 | 10.67 | C |
| ATOM | 25 | 6 | O | GLY D | 34 | −8.870 | 18.964 | −6.423 | 1.00 | 10.91 | O |
| ATOM | 25 | 7 | N | ILE D | 35 | −8.786 | 17.025 | −7.570 | 1.00 | 10.25 | N |
| ATOM | 25 | 8 | CA | ILE D | 35 | −7.779 | 17.483 | −8.534 | 1.00 | 10.07 | C |
| ATOM | 25 | 9 | CB | ILE D | 35 | −7.260 | 16.305 | −9.413 | 1.00 | 10.50 | C |
| ATOM | 26 | 0 | CG1 | ILE D | 35 | −6.609 | 15.238 | −8.515 | 1.00 | 11.70 | C |
| ATOM | 26 | 1 | CD1 | ILE D | 35 | −6.490 | 13.858 | −9.140 | 1.00 | 13.92 | C |
| ATOM | 26 | 2 | CG2 | ILE D | 35 | −6.278 | 16.808 | −10.486 | 1.00 | 10.43 | C |
| ATOM | 26 | 3 | C | ILE D | 35 | −8.338 | 18.628 | −9.387 | 1.00 | 9.27 | C |
| ATOM | 26 | 4 | O | ILE D | 35 | −7.695 | 19.672 | −9.540 | 1.00 | 9.31 | O |
| ATOM | 26 | 5 | N | LEU D | 36 | −9.543 | 18.437 | −9.918 | 1.00 | 8.55 | N |
| ATOM | 26 | 6 | CA | LEU D | 36 | −10.221 | 19.482 | −10.675 | 1.00 | 8.15 | C |
| ATOM | 26 | 7 | CB | LEU D | 36 | −11.600 | 19.005 | −11.137 | 1.00 | 7.94 | C |
| ATOM | 26 | 8 | CG | LEU D | 36 | −12.409 | 20.030 | −11.941 | 1.00 | 7.76 | C |
| ATOM | 26 | 9 | CD1 | LEU D | 36 | −11.736 | 20.338 | −13.273 | 1.00 | 9.06 | C |
| ATOM | 27 | 0 | CD2 | LEU D | 36 | −13.826 | 19.524 | −12.153 | 1.00 | 8.12 | C |
| ATOM | 27 | 1 | C | LEU D | 36 | −10.365 | 20.763 | −9.859 | 1.00 | 8.00 | C |
| ATOM | 27 | 2 | O | LEU D | 36 | −10.067 | 21.850 | −10.346 | 1.00 | 7.95 | O |
| ATOM | 27 | 3 | N | HIS D | 37 | −10.813 | 20.623 | −8.615 | 1.00 | 7.88 | N |
| ATOM | 27 | 4 | CA | HIS D | 37 | −11.017 | 21.765 | −7.728 | 1.00 | 7.67 | C |
| ATOM | 27 | 5 | CB | HIS D | 37 | −11.537 | 21.289 | −6.370 | 1.00 | 7.91 | C |
| ATOM | 27 | 6 | CG | HIS D | 37 | −12.073 | 22.389 | −5.506 | 1.00 | 8.93 | C |
| ATOM | 27 | 7 | ND1 | HIS D | 37 | −12.693 | 22.142 | −4.301 | 1.00 | 10.74 | N |
| ATOM | 27 | 8 | CE1 | HIS D | 37 | −13.059 | 23.287 | −3.754 | 1.00 | 11.34 | C |
| ATOM | 27 | 9 | NE2 | HIS D | 37 | −12.711 | 24.269 | −4.568 | 1.00 | 10.25 | N |
| ATOM | 28 | 0 | CD2 | HIS D | 37 | −12.098 | 23.733 | −5.675 | 1.00 | 9.47 | C |
| ATOM | 28 | 1 | C | HIS D | 37 | −9.733 | 22.584 | −7.557 | 1.00 | 7.84 | C |
| ATOM | 28 | 2 | O | HIS D | 37 | −9.754 | 23.811 | −7.686 | 1.00 | 7.58 | O |
| ATOM | 28 | 3 | N | LEU D | 38 | −8.609 | 21.910 | −7.296 | 1.00 | 7.82 | N |
| ATOM | 28 | 4 | CA | LEU D | 38 | −7.346 | 22.629 | −7.146 | 1.00 | 8.16 | C |
| ATOM | 28 | 5 | CB | LEU D | 38 | −6.203 | 21.688 | −6.754 | 1.00 | 8.57 | C |
| ATOM | 28 | 6 | CG | LEU D | 38 | −4.813 | 22.334 | −6.775 | 1.00 | 8.59 | C |
| ATOM | 28 | 7 | CD1 | LEU D | 38 | −4.660 | 23.393 | −5.669 | 1.00 | 9.69 | C |
| ATOM | 28 | 8 | CD2 | LEU D | 38 | −3.711 | 21.286 | −6.669 | 1.00 | 9.09 | C |
| ATOM | 28 | 9 | C | LEU D | 38 | −7.007 | 23.419 | −8.413 | 1.00 | 8.17 | C |
| ATOM | 29 | 0 | O | LEU D | 38 | −6.692 | 24.604 | −8.342 | 1.00 | 7.90 | O |
| ATOM | 29 | 1 | N | ILE D | 39 | −7.112 | 22.762 | −9.567 | 1.00 | 7.78 | N |
| ATOM | 29 | 2 | CA | ILE D | 39 | −6.812 | 23.396 | −10.853 | 1.00 | 8.03 | C |
| ATOM | 29 | 3 | CB | ILE D | 39 | −6.967 | 22.394 | −12.011 | 1.00 | 8.29 | C |
| ATOM | 29 | 4 | CG1 | ILE D | 39 | −5.899 | 21.299 | −11.894 | 1.00 | 8.04 | C |
| ATOM | 29 | 5 | CD1 | ILE D | 39 | −6.183 | 20.035 | −12.703 | 1.00 | 9.57 | C |
| ATOM | 29 | 6 | CG2 | ILE D | 39 | −6.890 | 23.113 | −13.364 | 1.00 | 8.35 | C |
| ATOM | 29 | 7 | C | ILE D | 39 | −7.689 | 24.634 | −11.075 | 1.00 | 7.86 | C |
| ATOM | 29 | 8 | O | ILE D | 39 | −7.182 | 25.720 | −11.391 | 1.00 | 8.11 | O |
| ATOM | 29 | 9 | N | LEU D | 40 | −8.996 | 24.481 | −10.869 | 1.00 | 7.83 | N |
| ATOM | 30 | 0 | CA | LEU D | 40 | −9.935 | 25.581 | −11.057 | 1.00 | 7.64 | C |
| ATOM | 30 | 1 | CB | LEU D | 40 | −11.375 | 25.100 | −10.891 | 1.00 | 7.85 | C |
| ATOM | 30 | 2 | CG | LEU D | 40 | −11.878 | 24.088 | −11.915 | 1.00 | 7.67 | C |
| ATOM | 30 | 3 | CD1 | LEU D | 40 | −13.330 | 23.751 | −11.606 | 1.00 | 8.52 | C |
| ATOM | 30 | 4 | CD2 | LEU D | 40 | −11.715 | 24.598 | −13.355 | 1.00 | 8.98 | C |
| ATOM | 30 | 5 | C | LEU D | 40 | −9.664 | 26.731 | −10.100 | 1.00 | 7.79 | C |
| ATOM | 30 | 6 | O | LEU D | 40 | −9.746 | 27.894 | −10.484 | 1.00 | 7.75 | O |
| ATOM | 30 | 7 | N | TRP D | 41 | −9.330 | 26.401 | −8.855 | 1.00 | 8.04 | N |
| ATOM | 30 | 8 | CA | TRP D | 41 | −9.084 | 27.432 | −7.858 | 1.00 | 8.64 | C |
| ATOM | 30 | 9 | CB | TRP D | 41 | −9.009 | 26.840 | −6.450 | 1.00 | 9.15 | C |
| ATOM | 31 | 0 | CG | TRP D | 41 | −8.705 | 27.859 | −5.409 | 1.00 | 9.55 | C |
| ATOM | 31 | 1 | CD1 | TRP D | 41 | −9.578 | 28.749 | −4.840 | 1.00 | 10.58 | C |
| ATOM | 31 | 2 | NE1 | TRP D | 41 | −8.917 | 29.538 | −3.927 | 1.00 | 10.41 | N |
| ATOM | 31 | 3 | CE2 | TRP D | 41 | −7.598 | 29.162 | −3.893 | 1.00 | 9.87 | C |
| ATOM | 31 | 4 | CD2 | TRP D | 41 | −7.433 | 28.107 | −4.816 | 1.00 | 9.53 | C |
| ATOM | 31 | 5 | CE3 | TRP D | 41 | −6.164 | 27.536 | −4.972 | 1.00 | 10.54 | C |
| ATOM | 31 | 6 | CZ3 | TRP D | 41 | −5.116 | 28.029 | −4.214 | 1.00 | 9.56 | C |
| ATOM | 31 | 7 | CH2 | TRP D | 41 | −5.310 | 29.080 | −3.303 | 1.00 | 10.18 | C |
| ATOM | 31 | 8 | CZ2 | TRP D | 41 | −6.539 | 29.658 | −3.127 | 1.00 | 9.91 | C |
| ATOM | 31 | 9 | C | TRP D | 41 | −7.834 | 28.239 | −8.211 | 1.00 | 8.65 | C |
| ATOM | 32 | 0 | O | TRP D | 41 | −7.841 | 29.463 | −8.124 | 1.00 | 8.89 | O |
| ATOM | 32 | 1 | N | ILE D | 42 | −6.777 | 27.549 | −8.627 | 1.00 | 8.89 | N |
| ATOM | 32 | 2 | CA | ILE D | 42 | −5.571 | 28.225 | −9.114 | 1.00 | 9.26 | C |
| ATOM | 32 | 3 | CB | ILE D | 42 | −4.478 | 27.211 | −9.542 | 1.00 | 9.25 | C |
| ATOM | 32 | 4 | CG1 | ILE D | 42 | −3.932 | 26.481 | −8.307 | 1.00 | 9.52 | C |
| ATOM | 32 | 5 | CD1 | ILE D | 42 | −3.082 | 25.269 | −8.609 | 1.00 | 11.75 | C |
| ATOM | 32 | 6 | CG2 | ILE D | 42 | −3.351 | 27.908 | −10.321 | 1.00 | 10.00 | C |
| ATOM | 32 | 7 | C | ILE D | 42 | −5.907 | 29.199 | −10.255 | 1.00 | 9.16 | C |
| ATOM | 32 | 8 | O | ILE D | 42 | −5.501 | 30.361 | −10.225 | 1.00 | 8.97 | O |
| ATOM | 32 | 9 | N | LEU D | 43 | −6.663 | 28.725 | −11.242 | 1.00 | 9.42 | N |
| ATOM | 33 | 0 | CA | LEU D | 43 | −7.027 | 29.559 | −12.393 | 1.00 | 10.26 | C |
| ATOM | 33 | 1 | CB | LEU D | 43 | −7.761 | 28.730 | −13.450 | 1.00 | 10.05 | C |
| ATOM | 33 | 2 | CG | LEU D | 43 | −6.926 | 27.625 | −14.099 | 1.00 | 9.36 | C |
| ATOM | 33 | 3 | CD1 | LEU D | 43 | −7.819 | 26.667 | −14.890 | 1.00 | 10.41 | C |
| ATOM | 33 | 4 | CD2 | LEU D | 43 | −5.792 | 28.191 | −14.976 | 1.00 | 9.38 | C |
| ATOM | 33 | 5 | C | LEU D | 43 | −7.857 | 30.770 | −11.981 | 1.00 | 11.14 | C |
| ATOM | 33 | 6 | O | LEU D | 43 | −7.675 | 31.869 | −12.512 | 1.00 | 11.19 | O |
| ATOM | 33 | 7 | N | ASP D | 44 | −8.754 | 30.561 | −11.024 | 1.00 | 11.86 | N |
| ATOM | 33 | 8 | CA | ASP D | 44 | −9.537 | 31.643 | −10.430 | 1.00 | 13.04 | C |
| ATOM | 33 | 9 | CB | ASP D | 44 | −10.528 | 31.077 | −9.405 | 1.00 | 13.50 | C |
| ATOM | 34 | 0 | CG | ASP D | 44 | −11.297 | 32.165 | −8.676 | 1.00 | 14.98 | C |
| ATOM | 34 | 1 | OD1 | ASP D | 44 | −12.165 | 32.801 | −9.308 | 1.00 | 18.01 | O |
| ATOM | 34 | 2 | OD2 | ASP D | 44 | −11.024 | 32.387 | −7.476 | 1.00 | 18.29 | O |
| ATOM | 34 | 3 | C | ASP D | 44 | −8.639 | 32.693 | −9.760 | 1.00 | 13.66 | C |
| ATOM | 34 | 4 | O | ASP D | 44 | −8.861 | 33.897 | −9.924 | 1.00 | 13.54 | O |
| ATOM | 34 | 5 | N | ARG D | 45 | −7.627 | 32.238 | −9.021 | 1.00 | 14.20 | N |
| ATOM | 34 | 6 | CA | ARG D | 45 | −6.727 | 33.151 | −8.309 | 1.00 | 14.94 | C |
| ATOM | 34 | 7 | CB | ARG D | 45 | −5.910 | 32.427 | −7.229 | 1.00 | 15.28 | C |
| ATOM | 34 | 8 | CG | ARG D | 45 | −6.738 | 31.737 | −6.132 | 1.00 | 17.17 | C |
| ATOM | 34 | 9 | CD | ARG D | 45 | −8.061 | 32.452 | −5.808 | 1.00 | 21.37 | C |
| ATOM | 35 | 0 | NE | ARG D | 45 | −7.874 | 33.700 | −5.075 | 1.00 | 25.02 | N |
| ATOM | 35 | 1 | CZ | ARG D | 45 | −8.758 | 34.693 | −5.043 | 1.00 | 26.50 | C |
| ATOM | 35 | 2 | NH1 | ARG D | 45 | −9.900 | 34.608 | −5.718 | 1.00 | 26.80 | N |
| ATOM | 35 | 3 | NH2 | ARG D | 45 | −8.492 | 35.786 | −4.341 | 1.00 | 28.14 | N |
| ATOM | 35 | 4 | C | ARG D | 45 | −5.811 | 33.918 | −9.260 | 1.00 | 15.14 | C |
| ATOM | 35 | 5 | O | ARG D | 45 | −5.458 | 35.066 | −8.991 | 1.00 | 15.68 | O |
| ATOM | 35 | 6 | N | LEU D | 46 | −5.443 | 33.289 | −10.371 | 1.00 | 15.11 | N |
| ATOM | 35 | 7 | CA | LEU D | 46 | −4.651 | 33.951 | −11.402 | 1.00 | 15.59 | C |
| ATOM | 35 | 8 | CB | LEU D | 46 | −4.133 | 32.934 | −12.420 | 1.00 | 15.43 | C |
| ATOM | 35 | 9 | CG | LEU D | 46 | −3.090 | 31.909 | −11.967 | 1.00 | 15.97 | C |
| ATOM | 36 | 0 | CD1 | LEU D | 46 | −2.799 | 30.942 | −13.106 | 1.00 | 17.01 | C |
| ATOM | 36 | 1 | CD2 | LEU D | 46 | −1.804 | 32.576 | −11.489 | 1.00 | 17.54 | C |
| ATOM | 36 | 2 | C | LEU D | 46 | −5.470 | 35.021 | −12.118 | 1.00 | 15.46 | C |
| ATOM | 36 | 3 | O | LEU D | 46 | −4.915 | 35.964 | −12.684 | 1.00 | 16.47 | O |
| ATOM | 36 | 4 | N | NH2 D | 47 | −6.729 | 34.972 | −12.173 | 1.00 | 15.05 | N |
| ATOM | 36 | 5 | BR4 | BRB E | 1 | −11.376 | 0.266 | 6.003 | 1.00 | 28.13 | BR |
| ATOM | 36 | 6 | C4 | BRB E | 1 | −12.591 | 1.709 | 6.100 | 1.00 | 22.67 | C |
| ATOM | 36 | 7 | C3 | BRB E | 1 | −13.296 | 2.103 | 4.966 | 1.00 | 23.40 | C |
| ATOM | 36 | 8 | C2 | BRB E | 1 | −14.194 | 3.166 | 5.044 | 1.00 | 23.01 | C |
| ATOM | 36 | 9 | C5 | BRB E | 1 | −12.767 | 2.361 | 7.316 | 1.00 | 22.89 | C |
| ATOM | 37 | 0 | C6 | BRB E | 1 | −13.659 | 3.423 | 7.392 | 1.00 | 22.63 | C |
| ATOM | 37 | 1 | C1 | BRB E | 1 | −14.371 | 3.824 | 6.260 | 1.00 | 21.65 | C |
| ATOM | 37 | 2 | C7 | BRB E | 1 | −15.346 | 4.972 | 6.359 | 1.00 | 17.72 | C |
| ATOM | 37 | 3 | O1 | BRB E | 1 | −15.204 | 5.800 | 5.201 | 1.00 | 14.13 | O |
| ATOM | 37 | 4 | N | PRO E | 25 | −16.126 | 5.246 | 7.321 | 1.00 | 13.87 | N |
| ATOM | 37 | 5 | CA | PRO E | 25 | −17.022 | 6.394 | 7.221 | 1.00 | 12.61 | C |
| ATOM | 37 | 6 | CB | PRO E | 25 | −17.781 | 6.346 | 8.547 | 1.00 | 12.76 | C |
| ATOM | 37 | 7 | CG | PRO E | 25 | −17.922 | 4.861 | 8.787 | 1.00 | 13.56 | C |
| ATOM | 37 | 8 | CD | PRO E | 25 | −16.634 | 4.246 | 8.283 | 1.00 | 13.40 | C |
| ATOM | 37 | 9 | C | PRO E | 25 | −16.305 | 7.735 | 7.026 | 1.00 | 12.15 | C |
| ATOM | 38 | 0 | O | PRO E | 25 | −16.830 | 8.603 | 6.327 | 1.00 | 12.07 | O |
| ATOM | 38 | 1 | N | LEU E | 26 | −15.116 | 7.885 | 7.611 | 1.00 | 1.22 | N |
| ATOM | 38 | 2 | CA | LEU E | 26 | −14.320 | 9.105 | 7.433 | 1.00 | 10.75 | C |
| ATOM | 38 | 3 | CB | LEU E | 26 | −13.006 | 9.016 | 8.214 | 1.00 | 10.89 | C |
| ATOM | 38 | 4 | CG | LEU E | 26 | −11.973 | 10.127 | 8.003 | 1.00 | 11.55 | C |
| ATOM | 38 | 5 | CD1 | LEU E | 26 | −12.496 | 11.488 | 8.463 | 1.00 | 12.36 | C |
| ATOM | 38 | 6 | CD2 | LEU E | 26 | −10.658 | 9.788 | 8.695 | 1.00 | 10.99 | C |
| ATOM | 38 | 7 | C | LEU E | 26 | −14.034 | 9.389 | 5.957 | 1.00 | 10.18 | C |
| ATOM | 38 | 8 | O | LEU E | 26 | −14.144 | 10.534 | 5.501 | 1.00 | 9.88 | O |
| ATOM | 38 | 9 | N | VAL E | 27 | −13.632 | 8.348 | 5.233 | 1.00 | 10.04 | N |
| ATOM | 39 | 0 | CA | VAL E | 27 | −13.288 | 8.472 | 3.815 | 1.00 | 10.22 | C |
| ATOM | 39 | 1 | CB | VAL E | 27 | −12.523 | 7.222 | 3.301 | 1.00 | 10.38 | C |
| ATOM | 39 | 2 | CG1 | VAL E | 27 | −12.263 | 7.317 | 1.795 | 1.00 | 11.45 | C |
| ATOM | 39 | 3 | CG2 | VAL E | 27 | −11.211 | 7.061 | 4.060 | 1.00 | 10.86 | C |
| ATOM | 39 | 4 | C | VAL E | 27 | −14.532 | 8.740 | 2.967 | 1.00 | 9.88 | C |
| ATOM | 39 | 5 | O | VAL E | 27 | −14.504 | 9.584 | 2.068 | 1.00 | 9.75 | O |
| ATOM | 39 | 6 | N | VAL E | 28 | −15.624 | 8.034 | 3.265 | 1.00 | 9.43 | N |
| ATOM | 39 | 7 | CA | VAL E | 28 | −16.903 | 8.257 | 2.587 | 1.00 | 9.22 | C |
| ATOM | 39 | 8 | CB | VAL E | 28 | −17.973 | 7.250 | 3.070 | 1.00 | 9.27 | C |
| ATOM | 39 | 9 | CG1 | VAL E | 28 | −19.343 | 7.567 | 2.474 | 1.00 | 9.20 | C |
| ATOM | 40 | 0 | CG2 | VAL E | 28 | −17.547 | 5.833 | 2.715 | 1.00 | 9.80 | C |
| ATOM | 40 | 1 | C | VAL E | 28 | −17.364 | 9.701 | 2.798 | 1.00 | 8.81 | C |
| ATOM | 40 | 2 | O | VAL E | 28 | −17.713 | 10.392 | 1.841 | 1.00 | 8.14 | O |
| ATOM | 40 | 3 | N | ALA E | 29 | −17.332 | 10.150 | 4.054 | 1.00 | 8.54 | N |
| ATOM | 40 | 4 | CA | ALA E | 29 | −17.688 | 11.524 | 4.398 | 1.00 | 8.70 | C |
| ATOM | 40 | 5 | CB | ALA E | 29 | −17.616 | 11.741 | 5.904 | 1.00 | 8.63 | C |
| ATOM | 40 | 6 | C | ALA E | 29 | −16.814 | 12.543 | 3.673 | 1.00 | 8.57 | C |
| ATOM | 40 | 7 | O | ALA E | 29 | −17.333 | 13.483 | 3.067 | 1.00 | 8.87 | O |
| ATOM | 40 | 8 | N | ALA E | 30 | −15.497 | 12.351 | 3.716 | 1.00 | 8.41 | N |
| ATOM | 40 | 9 | CA | ALA E | 30 | −14.573 | 13.275 | 3.058 | 1.00 | 8.45 | C |
| ATOM | 41 | 0 | CB | ALA E | 30 | −13.122 | 12.900 | 3.359 | 1.00 | 8.83 | C |
| ATOM | 41 | 1 | C | ALA E | 30 | −14.816 | 13.348 | 1.552 | 1.00 | 8.76 | C |
| ATOM | 41 | 2 | O | ALA E | 30 | −14.722 | 14.424 | 0.960 | 1.00 | 8.84 | O |
| ATOM | 41 | 3 | N | SER E | 31 | −15.144 | 12.203 | 0.951 | 1.00 | 8.77 | N |
| ATOM | 41 | 4 | CA | SER E | 31 | −15.432 | 12.130 | −0.482 | 1.00 | 9.09 | C |
| ATOM | 41 | 5 | CB | SER E | 31 | −15.579 | 10.674 | −0.917 | 1.00 | 9.09 | C |
| ATOM | 41 | 6 | OG | SER E | 31 | −14.346 | 9.988 | −0.788 | 1.00 | 9.32 | O |
| ATOM | 41 | 7 | C | SER E | 31 | −16.679 | 12.932 | −0.848 | 1.00 | 9.05 | C |
| ATOM | 41 | 8 | O | SER E | 31 | −16.666 | 13.727 | −1.790 | 1.00 | 9.12 | O |
| ATOM | 41 | 9 | N | ILE E | 32 | −17.745 | 12.732 | −0.078 | 1.00 | 8.73 | N |
| ATOM | 42 | 0 | CA | ILE E | 32 | −18.977 | 13.493 | −0.251 | 1.00 | 9.09 | C |
| ATOM | 42 | 1 | CB | ILE E | 32 | −20.042 | 13.011 | 0.749 | 1.00 | 9.04 | C |
| ATOM | 42 | 2 | CG1 | ILE E | 32 | −20.434 | 11.569 | 0.406 | 1.00 | 9.47 | C |
| ATOM | 42 | 3 | CD1 | ILE E | 32 | −21.120 | 10.814 | 1.526 | 1.00 | 10.13 | C |
| ATOM | 42 | 4 | CG2 | ILE E | 32 | −21.255 | 13.954 | 0.757 | 1.00 | 10.00 | C |
| ATOM | 42 | 5 | C | ILE E | 32 | −18.706 | 14.989 | −0.080 | 1.00 | 9.18 | C |
| ATOM | 42 | 6 | O | ILE E | 32 | −19.161 | 15.817 | −0.873 | 1.00 | 9.17 | O |
| ATOM | 42 | 7 | N | ILE E | 33 | −17.939 | 15.319 | 0.955 | 1.00 | 9.50 | N |
| ATOM | 42 | 8 | CA | ILE E | 33 | −17.586 | 16.710 | 1.258 | 1.00 | 10.31 | C |
| ATOM | 42 | 9 | CB | ILE E | 33 | −16.895 | 16.812 | 2.647 | 1.00 | 10.67 | C |
| ATOM | 43 | 0 | CG1 | ILE E | 33 | −17.920 | 16.576 | 3.775 | 1.00 | 11.36 | C |
| ATOM | 43 | 1 | CD1 | ILE E | 33 | −19.215 | 17.365 | 3.660 | 1.00 | 13.72 | C |
| ATOM | 43 | 2 | CG2 | ILE E | 33 | −16.141 | 18.137 | 2.825 | 1.00 | 11.10 | C |
| ATOM | 43 | 3 | C | ILE E | 33 | −16.760 | 17.362 | 0.146 | 1.00 | 10.44 | C |
| ATOM | 43 | 4 | O | ILE E | 33 | −16.993 | 18.523 | −0.197 | 1.00 | 10.27 | O |
| ATOM | 43 | 5 | N | GLY E | 34 | −15.809 | 16.622 | −0.422 | 1.00 | 10.68 | N |
| ATOM | 43 | 6 | CA | GLY E | 34 | −15.018 | 17.139 | −1.554 | 1.00 | 11.11 | C |
| ATOM | 43 | 7 | C | GLY E | 34 | −15.922 | 17.538 | −2.721 | 1.00 | 11.08 | C |
| ATOM | 43 | 8 | O | GLY E | 34 | −15.751 | 18.607 | −3.318 | 1.00 | 10.94 | O |
| ATOM | 43 | 9 | N | ILE E | 35 | −16.890 | 16.679 | −3.028 | 1.00 | 10.71 | N |
| ATOM | 44 | 0 | CA | ILE E | 35 | −17.823 | 16.909 | −4.123 | 1.00 | 10.76 | C |
| ATOM | 44 | 1 | CB | ILE E | 35 | −18.647 | 15.631 | −4.428 | 1.00 | 10.95 | C |
| ATOM | 44 | 2 | CG1 | ILE E | 35 | −17.712 | 14.525 | −4.952 | 1.00 | 10.60 | C |
| ATOM | 44 | 3 | CD1 | ILE E | 35 | −18.284 | 13.115 | −4.931 | 1.00 | 11.86 | C |
| ATOM | 44 | 4 | CG2 | ILE E | 35 | −19.770 | 15.924 | −5.429 | 1.00 | 11.51 | C |
| ATOM | 44 | 5 | C | ILE E | 35 | −18.716 | 18.113 | −3.817 | 1.00 | 10.04 | C |
| ATOM | 44 | 6 | O | ILE E | 35 | −18.868 | 19.017 | −4.652 | 1.00 | 10.04 | O |
| ATOM | 44 | 7 | N | LEU E | 36 | −19.278 | 18.138 | −2.612 | 1.00 | 9.27 | N |
| ATOM | 44 | 8 | CA | LEU E | 36 | −20.070 | 19.278 | −2.168 | 1.00 | 8.76 | C |
| ATOM | 44 | 9 | CB | LEU E | 36 | −20.629 | 19.024 | −0.769 | 1.00 | 8.68 | C |
| ATOM | 45 | 0 | CG | LEU E | 36 | −21.414 | 20.159 | −0.100 | 1.00 | 8.95 | C |
| ATOM | 45 | 1 | CD1 | LEU E | 36 | −22.764 | 20.380 | −0.784 | 1.00 | 9.44 | C |
| ATOM | 45 | 2 | CD2 | LEU E | 36 | −21.603 | 19.861 | 1.381 | 1.00 | 8.73 | C |
| ATOM | 45 | 3 | C | LEU E | 36 | −19.264 | 20.585 | −2.205 | 1.00 | 8.57 | C |
| ATOM | 45 | 4 | O | LEU E | 36 | −19.768 | 21.617 | −2.644 | 1.00 | 8.42 | O |
| ATOM | 45 | 5 | N | HIS E | 37 | −18.014 | 20.535 | −1.752 | 1.00 | 8.45 | N |
| ATOM | 45 | 6 | CA | HIS E | 37 | −17.165 | 21.723 | −1.727 | 1.00 | 8.78 | C |
| ATOM | 45 | 7 | CB | HIS E | 37 | −15.793 | 21.397 | −1.123 | 1.00 | 9.12 | C |
| ATOM | 45 | 8 | CG | HIS E | 37 | −14.966 | 22.604 | −0.794 | 1.00 | 10.22 | C |
| ATOM | 45 | 9 | ND1 | HIS E | 37 | −13.759 | 22.513 | −0.134 | 1.00 | 11.26 | N |
| ATOM | 46 | 0 | CE1 | HIS E | 37 | −13.251 | 23.723 | 0.019 | 1.00 | 11.19 | C |
| ATOM | 46 | 1 | NE2 | HIS E | 37 | −14.092 | 24.599 | −0.502 | 1.00 | 10.18 | N |
| ATOM | 46 | 2 | CD2 | HIS E | 37 | −15.173 | 23.926 | −1.016 | 1.00 | 10.40 | C |
| ATOM | 46 | 3 | C | HIS E | 37 | −17.002 | 22.296 | −3.138 | 1.00 | 8.75 | C |
| ATOM | 46 | 4 | O | HIS E | 37 | −17.153 | 23.507 | −3.340 | 1.00 | 8.31 | O |
| ATOM | 46 | 5 | N | LEU E | 38 | −16.723 | 21.436 | −4.116 | 1.00 | 8.62 | N |
| ATOM | 46 | 6 | CA | LEU E | 38 | −16.585 | 21.913 | −5.495 | 1.00 | 9.29 | C |
| ATOM | 46 | 7 | CB | LEU E | 38 | −16.131 | 20.799 | −6.453 | 1.00 | 9.52 | C |
| ATOM | 46 | 8 | CG | LEU E | 38 | −16.009 | 21.218 | −7.929 | 1.00 | 10.79 | C |
| ATOM | 46 | 9 | CD1 | LEU E | 38 | −14.897 | 22.235 | −8.134 | 1.00 | 11.09 | C |
| ATOM | 47 | 0 | CD2 | LEU E | 38 | −15.835 | 20.046 | −8.875 | 1.00 | 11.18 | C |
| ATOM | 47 | 1 | C | LEU E | 38 | −17.885 | 22.565 | −5.987 | 1.00 | 8.71 | C |
| ATOM | 47 | 2 | O | LEU E | 38 | −17.867 | 23.669 | −6.549 | 1.00 | 8.54 | O |
| ATOM | 47 | 3 | N | ILE E | 39 | −19.012 | 21.892 | −5.761 | 1.00 | 8.51 | N |
| ATOM | 47 | 4 | CA | ILE E | 39 | −20.308 | 22.428 | −6.184 | 1.00 | 8.35 | C |
| ATOM | 47 | 5 | CB | ILE E | 39 | −21.458 | 21.463 | −5.833 | 1.00 | 8.54 | C |
| ATOM | 47 | 6 | CG1 | ILE E | 39 | −21.348 | 20.200 | −6.698 | 1.00 | 8.95 | C |
| ATOM | 47 | 7 | CD1 | ILE E | 39 | −22.215 | 19.028 | −6.237 | 1.00 | 10.30 | C |
| ATOM | 47 | 8 | CG2 | ILE E | 39 | −22.819 | 22.148 | −6.013 | 1.00 | 8.32 | C |
| ATOM | 47 | 9 | C | ILE E | 39 | −20.556 | 23.811 | −5.581 | 1.00 | 8.47 | C |
| ATOM | 48 | 0 | O | ILE E | 39 | −20.881 | 24.753 | −6.296 | 1.00 | 8.00 | O |
| ATOM | 48 | 1 | N | LEU E | 40 | −20.388 | 23.924 | −4.263 | 1.00 | 8.27 | N |
| ATOM | 48 | 2 | CA | LEU E | 40 | −20.611 | 25.190 | −3.561 | 1.00 | 8.50 | C |
| ATOM | 48 | 3 | CB | LEU E | 40 | −20.451 | 25.009 | −2.051 | 1.00 | 8.52 | C |
| ATOM | 48 | 4 | CG | LEU E | 40 | −21.449 | 24.087 | −1.358 | 1.00 | 8.73 | C |
| ATOM | 48 | 5 | CD1 | LEU E | 40 | −21.095 | 23.969 | 0.120 | 1.00 | 8.89 | C |
| ATOM | 48 | 6 | CD2 | LEU E | 40 | −22.879 | 24.598 | −1.544 | 1.00 | 9.69 | C |
| ATOM | 48 | 7 | C | LEU E | 40 | −19.677 | 26.286 | −4.048 | 1.00 | 8.51 | C |
| ATOM | 48 | 8 | O | LEU E | 40 | −20.094 | 27.436 | −4.223 | 1.00 | 8.77 | O |
| ATOM | 48 | 9 | N | TRP E | 41 | −18.415 | 25.925 | −4.267 | 1.00 | 8.76 | N |
| ATOM | 49 | 0 | CA | TRP E | 41 | −17.415 | 26.893 | −4.702 | 1.00 | 9.11 | C |
| ATOM | 49 | 1 | CB | TRP E | 41 | −16.006 | 26.305 | −4.645 | 1.00 | 9.52 | C |
| ATOM | 49 | 2 | CG | TRP E | 41 | −14.955 | 27.261 | −5.114 | 1.00 | 10.01 | C |
| ATOM | 49 | 3 | CD1 | TRP E | 41 | −14.414 | 28.302 | −4.409 | 1.00 | 10.53 | C |
| ATOM | 49 | 4 | NE1 | TRP E | 41 | −13.469 | 28.956 | −5.175 | 1.00 | 10.03 | N |
| ATOM | 49 | 5 | CE2 | TRP E | 41 | −13.395 | 28.343 | −6.400 | 1.00 | 9.99 | C |
| ATOM | 49 | 6 | CD2 | TRP E | 41 | −14.317 | 27.270 | −6.399 | 1.00 | 10.01 | C |
| ATOM | 49 | 7 | CE3 | TRP E | 41 | −14.435 | 26.475 | −7.549 | 1.00 | 10.38 | C |
| ATOM | 49 | 8 | CZ3 | TRP E | 41 | −13.638 | 26.775 | −8.651 | 1.00 | 10.49 | C |
| ATOM | 49 | 9 | CH2 | TRP E | 41 | −12.732 | 27.849 | −8.620 | 1.00 | 9.97 | C |
| ATOM | 50 | 0 | CZ2 | TRP E | 41 | −12.597 | 28.642 | −7.507 | 1.00 | 9.65 | C |
| ATOM | 50 | 1 | C | TRP E | 41 | −17.732 | 27.425 | −6.096 | 1.00 | 9.27 | C |
| ATOM | 50 | 2 | O | TRP E | 41 | −17.658 | 28.627 | −6.324 | 1.00 | 9.11 | O |
| ATOM | 50 | 3 | N | ILE E | 42 | −18.118 | 26.539 | −7.010 | 1.00 | 9.72 | N |
| ATOM | 50 | 4 | CA | ILE E | 42 | −18.542 | 26.970 | −8.350 | 1.00 | 10.01 | C |
| ATOM | 50 | 5 | CB | ILE E | 42 | −18.848 | 25.773 | −9.282 | 1.00 | 10.39 | C |
| ATOM | 50 | 6 | CG1 | ILE E | 42 | −17.558 | 25.000 | −9.580 | 1.00 | 9.66 | C |
| ATOM | 50 | 7 | CD1 | ILE E | 42 | −17.764 | 23.668 | −10.294 | 1.00 | 10.24 | C |
| ATOM | 50 | 8 | CG2 | ILE E | 42 | −19.478 | 26.249 | −10.603 | 1.00 | 10.74 | C |
| ATOM | 50 | 9 | C | ILE E | 42 | −19.733 | 27.930 | −8.242 | 1.00 | 10.27 | C |
| 1.00 | 51 | 0 | O | ILE E | 42 | −19.725 | 29.009 | −8.843 | 1.00 | 10.27 | O |
| ATOM | 51 | 1 | N | LEU E | 43 | −20.733 | 27.550 | −7.448 | 1.00 | 10.18 | N |
| ATOM | 51 | 2 | CA | LEU E | 43 | −21.926 | 28.390 | −7.261 | 1.00 | 10.64 | C |
| ATOM | 51 | 3 | CB | LEU E | 43 | −22.977 | 27.665 | −6.419 | 1.00 | 10.38 | C |
| ATOM | 51 | 4 | CG | LEU E | 43 | −23.611 | 26.447 | −7.096 | 1.00 | 9.52 | C |
| ATOM | 51 | 5 | CD1 | LEU E | 43 | −24.373 | 25.597 | −6.082 | 1.00 | 9.33 | C |
| ATOM | 51 | 6 | CD2 | LEU E | 43 | −24.519 | 26.857 | −8.265 | 1.00 | 10.28 | C |
| ATOM | 51 | 7 | C | LEU E | 43 | −21.588 | 29.753 | −6.658 | 1.00 | 11.39 | C |
| ATOM | 51 | 8 | O | LEU E | 43 | −22.153 | 30.775 | −7.062 | 1.00 | 11.87 | O |
| ATOM | 51 | 9 | N | ASP E | 44 | −20.653 | 29.759 | −5.711 | 1.00 | 12.18 | N |
| ATOM | 52 | 0 | CA | ASP E | 44 | −20.171 | 30.992 | −5.099 | 1.00 | 13.45 | C |
| ATOM | 52 | 1 | CB | ASP E | 44 | −19.220 | 30.683 | −3.941 | 1.00 | 13.97 | C |
| ATOM | 52 | 2 | CG | ASP E | 44 | −18.628 | 31.936 | −3.328 | 1.00 | 15.83 | C |
| ATOM | 52 | 3 | OD1 | ASP E | 44 | −19.373 | 32.691 | −2.675 | 1.00 | 19.22 | O |
| ATOM | 52 | 4 | OD2 | ASP E | 44 | −17.417 | 32.169 | −3.520 | 1.00 | 19.69 | O |
| ATOM | 52 | 5 | C | ASP E | 44 | −19.482 | 31.904 | −6.120 | 1.00 | 13.74 | C |
| ATOM | 52 | 6 | O | ASP E | 44 | −19.717 | 33.117 | −6.129 | 1.00 | 13.99 | O |
| ATOM | 52 | 7 | N | ARG E | 45 | −18.646 | 31.329 | −6.982 | 1.00 | 14.35 | N |
| ATOM | 52 | 8 | CA | ARG E | 45 | −17.932 | 32.127 | −7.978 | 1.00 | 14.92 | C |
| ATOM | 52 | 9 | CB | ARG E | 45 | −16.765 | 31.354 | −8.610 | 1.00 | 14.98 | C |
| ATOM | 53 | 0 | CG | ARG E | 45 | −15.712 | 30.862 | −7.615 | 1.00 | 15.30 | C |
| ATOM | 53 | 1 | CD | ARG E | 45 | −15.341 | 31.918 | −6.566 | 1.00 | 16.21 | C |
| ATOM | 53 | 2 | NE | ARG E | 45 | −14.479 | 32.967 | −7.096 | 1.00 | 19.30 | N |
| ATOM | 53 | 3 | CZ | ARG E | 45 | −14.323 | 34.070 | −6.576 | 1.00 | 24.70 | C |
| ATOM | 53 | 4 | NH1 | ARG E | 45 | −14.944 | 34.366 | −5.438 | 1.00 | 25.92 | N |
| ATOM | 53 | 5 | NH2 | ARG E | 45 | −13.544 | 34.976 | −7.153 | 1.00 | 25.99 | N |
| ATOM | 53 | 6 | C | ARG E | 45 | −18.871 | 32.687 | −9.049 | 1.00 | 15.23 | C |
| ATOM | 53 | 7 | O | ARG E | 45 | −18.657 | 33.793 | −9.546 | 1.00 | 15.67 | O |
| ATOM | 53 | 8 | N | LEU E | 46 | −19.913 | 31.931 | −9.386 | 1.00 | 15.43 | N |
| ATOM | 53 | 9 | CA | LEU E | 46 | −20.923 | 32.394 | −10.348 | 1.00 | 15.51 | C |
| ATOM | 54 | 0 | CB | LEU E | 46 | −21.788 | 31.230 | −10.840 | 1.00 | 15.46 | C |
| ATOM | 54 | 1 | CG | LEU E | 46 | −21.156 | 30.157 | −11.736 | 1.00 | 15.58 | C |
| ATOM | 54 | 2 | CD1 | LEU E | 46 | −22.194 | 29.098 | −12.074 | 1.00 | 15.29 | C |
| ATOM | 54 | 3 | CD2 | LEU E | 46 | −20.558 | 30.751 | −13.018 | 1.00 | 15.11 | C |
| ATOM | 54 | 4 | C | LEU E | 46 | −21.798 | 33.493 | −9.757 | 1.00 | 15.68 | C |
| ATOM | 54 | 5 | O | LEU E | 46 | −22.396 | 34.285 | −10.496 | 1.00 | 16.33 | O |
| ATOM | 54 | 6 | N | NH2 E | 47 | −21.830 | 33.780 | −8.529 | 1.00 | 15.23 | N |
| ATOM | 54 | 7 | BR4 | BRB F | 1 | −3.398 | 0.486 | 3.631 | 1.00 | 35.49 | BR |
| ATOM | 54 | 8 | C4 | BRB F | 1 | −3.319 | 2.177 | 4.476 | 1.00 | 29.87 | C |
| ATOM | 54 | 9 | C3 | BRB F | 1 | −4.459 | 2.727 | 5.063 | 1.00 | 30.64 | C |
| ATOM | 55 | 0 | C2 | BRB F | 1 | −4.388 | 3.974 | 5.684 | 1.00 | 30.35 | C |
| ATOM | 55 | 1 | C5 | BRB F | 1 | −2.110 | 2.865 | 4.508 | 1.00 | 30.46 | C |
| ATOM | 55 | 2 | C6 | BRB F | 1 | −2.040 | 4.110 | 5.127 | 1.00 | 29.97 | C |
| ATOM | 55 | 3 | Cl | BRB F | 1 | −3.177 | 4.664 | 5.716 | 1.00 | 28.88 | C |
| ATOM | 55 | 4 | C7 | BRB F | 1 | −3.088 | 6.017 | 6.390 | 1.00 | 22.95 | C |
| ATOM | 55 | 5 | O1 | BRB F | 1 | −4.162 | 6.835 | 5.918 | 1.00 | 18.86 | O |
| ATOM | 55 | 6 | N | PRO F | 25 | −2.186 | 6.586 | 7.081 | 1.00 | 16.29 | N |
| ATOM | 55 | 7 | CA | PRO F | 25 | −2.277 | 7.931 | 7.632 | 1.00 | 14.26 | C |
| ATOM | 55 | 8 | CB | PRO F | 25 | −0.960 | 8.076 | 8.402 | 1.00 | 14.33 | C |
| ATOM | 55 | 9 | CG | PRO F | 25 | −0.705 | 6.692 | .902 | 1.00 | 15.17 | C |
| ATOM | 56 | 0 | CD | PRO F | 25 | −1.190 | 5.772 | 7.809 | 1.00 | 15.34 | C |
| ATOM | 56 | 1 | C | PRO F | 25 | −2.412 | 9.034 | 6.583 | 1.00 | 13.38 | C |
| ATOM | 56 | 2 | O | PRO F | 25 | −3.021 | 10.059 | 6.873 | 1.00 | 12.92 | O |
| ATOM | 56 | 3 | N | LEU F | 26 | −1.860 | 8.840 | 5.385 | 1.00 | 12.29 | N |
| ATOM | 56 | 4 | CA | LEU F | 26 | −2.001 | 9.858 | 4.342 | 1.00 | 11.95 | C |
| ATOM | 56 | 5 | CB | LEU F | 26 | −1.202 | 9.496 | 3.083 | 1.00 | 11.81 | C |
| ATOM | 56 | 6 | CG | LEU F | 26 | −1.469 | 10.335 | 1.822 | 1.00 | 11.75 | C |
| ATOM | 56 | 7 | CD1 | LEU F | 26 | −1.088 | 11.800 | 2.008 | 1.00 | 11.95 | C |
| ATOM | 56 | 8 | CD2 | LEU F | 26 | −0.748 | 9.746 | 0.616 | 1.00 | 12.35 | C |
| ATOM | 56 | 9 | C | VAL F | 27 | −3.473 | 10.081 | 3.993 | 1.00 | 11.58 | C |
| ATOM | 57 | 0 | O | VAL F | 27 | −3.932 | 11.224 | 3.905 | 1.00 | 11.74 | O |
| ATOM | 57 | 1 | N | VAL F | 27 | −4.199 | 8.983 | 3.798 | 1.00 | 11.50 | N |
| ATOM | 57 | 2 | CA | VAL F | 27 | −5.621 | 9.044 | 3.440 | 1.00 | 11.62 | C |
| ATOM | 57 | 3 | CB | VAL F | 27 | −6.145 | 7.665 | 2.958 | 1.00 | 12.09 | C |
| ATOM | 57 | 4 | CG1 | VAL F | 27 | −7.635 | 7.732 | 2.637 | 1.00 | 13.11 | C |
| ATOM | 57 | 5 | CG2 | VAL F | 27 | −5.367 | 7.208 | 1.731 | 1.00 | 12.84 | C |
| ATOM | 57 | 6 | C | VAL F | 27 | −6.470 | 9.600 | 4.592 | 1.00 | 11.18 | C |
| ATOM | 57 | 7 | O | VAL F | 27 | −7.360 | 10.433 | 4.370 | 1.00 | 11.16 | O |
| ATOM | 57 | 8 | N | VAL F | 28 | −6.181 | 9.157 | 5.818 | 1.00 | 10.54 | N |
| ATOM | 57 | 9 | CA | VAL F | 28 | −6.854 | 9.681 | 7.014 | 1.00 | 9.85 | C |
| ATOM | 58 | 0 | CB | VAL F | 28 | −6.355 | 8.970 | 8.311 | 1.00 | 10.06 | C |
| ATOM | 58 | 1 | CG1 | VAL F | 28 | −6.940 | 9.619 | 9.568 | 1.00 | 10.32 | C |
| ATOM | 58 | 2 | CG2 | VAL F | 28 | −6.698 | 7.486 | 8.256 | 1.00 | 10.37 | C |
| ATOM | 58 | 3 | C | VAL F | 28 | −6.644 | 11.192 | 7.105 | 1.00 | 9.52 | C |
| ATOM | 58 | 4 | O | VAL F | 28 | −7.609 | 11.957 | 7.216 | 1.00 | 9.09 | O |
| ATOM | 58 | 5 | N | ALA F | 29 | −5.383 | 11.619 | 7.025 | 1.00 | 8.99 | N |
| ATOM | 58 | 6 | CA | ALA F | 29 | −5.048 | 13.036 | 7.118 | 1.00 | 8.57 | C |
| ATOM | 58 | 7 | CB | ALA F | 29 | −3.538 | 13.235 | 7.113 | 1.00 | 8.71 | C |
| ATOM | 58 | 8 | C | ALA F | 29 | −5.715 | 13.869 | 6.019 | 1.00 | 8.41 | C |
| ATOM | 58 | 9 | O | ALA F | 29 | −6.252 | 14.940 | 6.302 | 1.00 | 8.16 | O |
| ATOM | 59 | 0 | N | ALA F | 30 | −5.709 | 13.365 | 4.784 | 1.00 | 8.31 | N |
| ATOM | 59 | 1 | CA | ALA F | 30 | −6.324 | 14.089 | 3.663 | 1.00 | 8.16 | C |
| ATOM | 59 | 2 | CB | ALA F | 30 | −6.012 | 13.403 | 2.342 | 1.00 | 8.80 | C |
| ATOM | 59 | 3 | C | ALA F | 30 | −7.833 | 14.218 | 3.859 | 1.00 | 8.31 | C |
| ATOM | 59 | 4 | O | ALA F | 30 | −8.429 | 15.248 | 3.530 | 1.00 | 8.45 | O |
| ATOM | 59 | 5 | N | SER F | 31 | −8.437 | 13.163 | 4.399 | 1.00 | 7.91 | N |
| ATOM | 59 | 6 | CA | SER F | 31 | −9.867 | 13.141 | 4.686 | 1.00 | 8.21 | C |
| ATOM | 59 | 7 | CB | SER F | 31 | −10.299 | 11.734 | 5.102 | 1.00 | 8.30 | C |
| ATOM | 59 | 8 | OG | SER F | 31 | −10.093 | 10.835 | 4.023 | 1.00 | 9.12 | O |
| ATOM | 59 | 9 | C | SER F | 31 | −10.222 | 14.171 | 5.753 | 1.00 | 8.11 | C |
| ATOM | 60 | 0 | O | SER F | 31 | −11.159 | 14.953 | 5.586 | 1.00 | 8.12 | O |
| ATOM | 60 | 1 | N | ILE F | 32 | −9.451 | 14.190 | 6.838 | 1.00 | 8.05 | N |
| ATOM | 60 | 2 | CA | ILE F | 32 | −9.626 | 15.197 | 7.883 | 1.00 | 7.87 | C |
| ATOM | 60 | 3 | CB | ILE F | 32 | −8.601 | 14.986 | 9.017 | 1.00 | 8.25 | C |
| ATOM | 60 | 4 | CG1 | ILE F | 32 | −8.879 | 13.649 | 9.709 | 1.00 | 8.15 | C |
| ATOM | 60 | 5 | CD1 | ILE F | 32 | −7.781 | 13.191 | 10.653 | 1.00 | 9.13 | C |
| ATOM | 60 | 6 | CG2 | ILE F | 32 | −8.625 | 16.155 | 10.009 | 1.00 | 7.36 | C |
| ATOM | 60 | 7 | C | ILE F | 32 | −9.499 | 16.601 | 7.293 | 1.00 | 8.46 | C |
| ATOM | 60 | 8 | O | ILE F | 32 | −10.317 | 17.483 | 7.570 | 1.00 | 7.99 | O |
| ATOM | 60 | 9 | N | ILE F | 33 | −8.474 | 16.789 | 6.469 | 1.00 | 8.27 | N |
| ATOM | 61 | 0 | CA | ILE F | 33 | −8.170 | 18.092 | 5.883 | 1.00 | 9.05 | C |
| ATOM | 61 | 1 | CB | ILE F | 33 | −6.748 | 18.095 | 5.263 | 1.00 | 9.26 | C |
| ATOM | 61 | 2 | CG1 | ILE F | 33 | −5.732 | 18.320 | 6.394 | 1.00 | 10.51 | C |
| ATOM | 61 | 3 | CD1 | ILE F | 33 | −4.331 | 17.806 | 6.125 | 1.00 | 13.12 | C |
| ATOM | 61 | 4 | CG2 | ILE F | 33 | −6.601 | 19.174 | 4.202 | 1.00 | 10.01 | C |
| ATOM | 61 | 5 | C | ILE F | 33 | −9.250 | 18.553 | 4.910 | 1.00 | 9.31 | C |
| ATOM | 61 | 6 | O | ILE F | 33 | −9.599 | 19.737 | 4.886 | 1.00 | 9.05 | O |
| ATOM | 61 | 7 | N | GLY F | 34 | −9.804 | 17.616 | 4.140 | 1.00 | 9.47 | N |
| ATOM | 61 | 8 | CA | GLY F | 34 | −10.916 | 17.937 | 3.233 | 1.00 | 9.72 | C |
| ATOM | 61 | 9 | C | GLY F | 34 | −12.126 | 18.483 | 4.002 | 1.00 | 9.66 | C |
| ATOM | 62 | 0 | O | GLY F | 34 | −12.755 | 19.461 | 3.578 | 1.00 | 9.90 | O |
| ATOM | 62 | 1 | N | ILE F | 35 | −12.440 | 17.854 | 5.136 | 1.00 | 9.12 | N |
| ATOM | 62 | 2 | CA | ILE F | 35 | −13.552 | 18.294 | 5.981 | 1.00 | 8.79 | C |
| ATOM | 62 | 3 | CB | ILE F | 35 | −13.895 | 17.244 | 7.075 | 1.00 | 8.84 | C |
| ATOM | 62 | 4 | CG1 | ILE F | 35 | −14.321 | 15.922 | 6.412 | 1.00 | 9.35 | C |
| ATOM | 62 | 5 | CD1 | ILE F | 35 | −14.490 | 14.750 | 7.359 | 1.00 | 9.16 | C |
| ATOM | 62 | 6 | CG2 | ILE F | 35 | −14.982 | 17.776 | 8.025 | 1.00 | 8.77 | C |
| ATOM | 62 | 7 | C | ILE F | 35 | −13.237 | 19.660 | 6.590 | 1.00 | 8.47 | C |
| ATOM | 62 | 8 | O | ILE F | 35 | −14.059 | 20.569 | 6.531 | 1.00 | 8.20 | O |
| ATOM | 62 | 9 | N | LEU F | 36 | −12.031 | 19.807 | 7.139 | 1.00 | 8.00 | N |
| ATOM | 63 | 0 | CA | LEU F | 36 | −11.598 | 21.088 | 7.700 | 1.00 | 8.31 | C |
| ATOM | 63 | 1 | CB | LEU F | 36 | −10.192 | 20.971 | 8.290 | 1.00 | 8.42 | C |
| ATOM | 63 | 2 | CG | LEU F | 36 | −9.599 | 22.256 | 8.873 | 1.00 | 8.29 | C |
| ATOM | 63 | 3 | CD1 | LEU F | 36 | −10.470 | 22.819 | 10.001 | 1.00 | 9.76 | C |
| ATOM | 63 | 4 | CD2 | LEU F | 36 | −8.174 | 21.987 | 9.355 | 1.00 | 9.59 | C |
| ATOM | 63 | 5 | C | LEU F | 36 | −11.643 | 22.205 | 6.654 | 1.00 | 8.11 | C |
| ATOM | 63 | 6 | O | LEU F | 36 | −12.116 | 23.306 | 6.930 | 1.00 | 7.75 | O |
| ATOM | 63 | 7 | N | HIS F | 37 | −11.147 | 21.912 | 5.456 | 1.00 | 8.31 | N |
| ATOM | 63 | 8 | CA | HIS F | 37 | −11.113 | 22.903 | 4.379 | 1.00 | 8.55 | C |
| ATOM | 63 | 9 | CB | HIS F | 37 | −10.477 | 22.304 | 3.124 | 1.00 | 8.58 | C |
| ATOM | 64 | 0 | CG | HIS F | 37 | −10.143 | 23.311 | 2.067 | 1.00 | 8.90 | C |
| ATOM | 64 | 1 | ND1 | HIS F | 37 | −9.445 | 22.976 | 0.928 | 1.00 | 9.80 | N |
| ATOM | 64 | 2 | CE1 | HIS F | 37 | −9.294 | 24.051 | 0.174 | 1.00 | 10.37 | C |
| ATOM | 64 | 3 | NE2 | HIS F | 37 | −9.858 | 25.074 | 0.789 | 1.00 | 9.61 | N |
| ATOM | 64 | 4 | CD2 | HIS F | 37 | −10.398 | 4.639 | 1.975 | 1.00 | 9.54 | C |
| ATOM | 64 | 5 | C | HIS F | 37 | −12.510 | 23.449 | 4.058 | 1.00 | 8.70 | C |
| ATOM | 64 | 6 | O | HIS F | 37 | −12.682 | 24.657 | 3.915 | 1.00 | 9.11 | O |
| ATOM | 64 | 7 | N | LEU F | 38 | −13.505 | 22.573 | 3.950 | 1.00 | 9.17 | N |
| ATOM | 64 | 8 | CA | LEU F | 38 | −14.873 | 23.030 | 3.695 | 1.00 | 9.30 | C |
| ATOM | 64 | 9 | CB | LEU F | 38 | −15.821 | 21.863 | 3.412 | 1.00 | 9.27 | C |
| ATOM | 65 | 0 | CG | LEU F | 38 | −17.267 | 22.292 | 3.110 | 1.00 | 10.27 | C |
| ATOM | 65 | 1 | CD1 | LEU F | 38 | −17.352 | 23.279 | 1.943 | 1.00 | 12.42 | C |
| ATOM | 65 | 2 | CD2 | LEU F | 38 | −18.142 | 21.100 | 2.836 | 1.00 | 12.82 | C |
| ATOM | 65 | 3 | C | LEU F | 38 | −15.412 | 23.884 | 4.843 | 1.00 | 9.36 | C |
| ATOM | 65 | 4 | O | LEU F | 38 | −16.006 | 24.940 | 4.608 | 1.00 | 9.33 | O |
| ATOM | 65 | 5 | N | ILE F | 39 | −15.200 | 23.433 | 6.079 | 1.00 | 9.37 | N |
| ATOM | 65 | 6 | CA | ILE F | 39 | −15.641 | 24.192 | 7.256 | 1.00 | 9.81 | C |
| ATOM | 65 | 7 | CB | ILE F | 39 | −15.282 | 23.468 | 8.578 | 1.00 | 9.75 | C |
| ATOM | 65 | 8 | CG1 | ILE F | 39 | −16.094 | 22.167 | 8.703 | 1.00 | 10.19 | C |
| ATOM | 65 | 9 | CD1 | ILE F | 39 | −15.648 | 21.236 | 9.845 | 1.00 | 10.37 | C |
| ATOM | 66 | 0 | CG2 | ILE F | 39 | −15.498 | 24.401 | 9.786 | 1.00 | 10.25 | C |
| ATOM | 66 | 1 | C | ILE F | 39 | −15.045 | 25.604 | 7.231 | 1.00 | 9.92 | C |
| ATOM | 66 | 2 | O | ILE F | 39 | −15.766 | 26.597 | 7.368 | 1.00 | 10.05 | O |
| ATOM | 66 | 3 | N | LEU F | 40 | −13.734 | 25.688 | 7.019 | 1.00 | 9.98 | N |
| ATOM | 66 | 4 | CA | LEU F | 40 | −13.054 | 26.976 | 6.956 | 1.00 | 10.32 | C |
| ATOM | 66 | 5 | CB | LEU F | 40 | −11.550 | 26.779 | 6.812 | 1.00 | 9.93 | C |
| ATOM | 66 | 6 | CG | LEU F | 40 | −10.774 | 26.251 | 8.012 | 1.00 | 10.45 | C |
| ATOM | 66 | 7 | CD1 | LEU F | 40 | −9.356 | 25.977 | 7.562 | 1.00 | 10.42 | C |
| ATOM | 66 | 8 | CD2 | LEU F | 40 | −10.801 | 27.254 | 9.174 | 1.00 | 10.90 | C |
| ATOM | 66 | 9 | C | LEU F | 40 | −13.568 | 27.849 | 5.818 | 1.00 | 11.01 | C |
| ATOM | 67 | 0 | O | LEU F | 40 | −13.771 | 29.053 | 5.996 | 1.00 | 11.60 | O |
| ATOM | 67 | 1 | N | TRP F | 41 | −13.791 | 27.241 | 4.653 | 1.00 | 11.31 | N |
| ATOM | 67 | 2 | CA | TRP F | 41 | −14.217 | 27.998 | 3.484 | 1.00 | 12.42 | C |
| ATOM | 67 | 3 | CB | TRP F | 41 | −14.103 | 27.157 | 2.203 | 1.00 | 12.47 | C |
| ATOM | 67 | 4 | CG | TRP F | 41 | −14.564 | 27.901 | 0.996 | 1.00 | 12.72 | C |
| ATOM | 67 | 5 | CD1 | TRP F | 41 | −13.857 | 28.829 | 0.282 | 1.00 | 12.92 | C |
| ATOM | 67 | 6 | NE1 | TRP F | 41 | −14.623 | 29.314 | −0.752 | 1.00 | 12.48 | N |
| ATOM | 67 | 7 | CE2 | TRP F | 41 | −15.851 | 28.705 | −0.717 | 1.00 | 12.67 | C |
| ATOM | 67 | 8 | CD2 | TRP F | 41 | −15.851 | 27.811 | 0.376 | 1.00 | 12.66 | C |
| ATOM | 67 | 9 | CE3 | TRP F | 41 | −17.002 | 27.053 | 0.635 | 1.00 | 13.18 | C |
| ATOM | 68 | 0 | CZ3 | TRP F | 41 | −18.105 | 27.212 | −0.195 | 1.00 | 13.05 | C |
| ATOM | 68 | 1 | CH2 | TRP F | 41 | −18.076 | 28.112 | −1.277 | 1.00 | 13.07 | C |
| ATOM | 68 | 2 | CZ2 | TRP F | 41 | −16.964 | 28.865 | −1.554 | 1.00 | 13.19 | C |
| ATOM | 68 | 3 | C | TRP F | 41 | −15.626 | 28.573 | 3.674 | 1.00 | 12.80 | C |
| ATOM | 68 | 4 | O | TRP F | 41 | −15.861 | 29.751 | 3.388 | 1.00 | 13.33 | O |
| ATOM | 68 | 5 | N | ILE F | 42 | −16.550 | 27.757 | 4.178 | 1.00 | 13.66 | N |
| ATOM | 68 | 6 | CA | ILE F | 42 | −17.910 | 28.231 | 4.476 | 1.00 | 14.45 | C |
| ATOM | 68 | 7 | CB | ILE F | 42 | −18.824 | 27.106 | 5.014 | 1.00 | 14.42 | C |
| ATOM | 68 | 8 | CG1 | ILE F | 42 | −19.062 | 26.051 | 3.929 | 1.00 | 14.19 | C |
| ATOM | 68 | 9 | CD1 | ILE F | 42 | −19.794 | 24.797 | 4.408 | 1.00 | 14.34 | C |
| ATOM | 69 | 0 | CG2 | ILE F | 42 | −20.165 | 27.679 | 5.495 | 1.00 | 14.62 | C |
| ATOM | 69 | 1 | C | ILE F | 42 | −17.865 | 29.408 | 5.458 | 1.00 | 15.26 | C |
| ATOM | 69 | 2 | O | ILE F | 42 | −18.486 | 30.445 | 5.219 | 1.00 | 15.51 | O |
| ATOM | 69 | 3 | N | LEU F | 43 | −17.109 | 29.249 | 6.545 | 1.00 | 16.18 | N |
| ATOM | 69 | 4 | CA | LEU F | 43 | −16.977 | 30.304 | 7.551 | 1.00 | 17.56 | C |
| ATOM | 69 | 5 | CB | LEU F | 43 | −16.218 | 29.798 | 8.782 | 1.00 | 17.68 | C |
| ATOM | 69 | 6 | CG | LEU F | 43 | −16.951 | 28.747 | 9.619 | 1.00 | 17.93 | C |
| ATOM | 69 | 7 | CD1 | LEU F | 43 | −16.031 | 28.179 | 10.699 | 1.00 | 17.90 | C |
| ATOM | 69 | 8 | CD2 | LEU F | 43 | −18.231 | 29.308 | 10.237 | 1.00 | 19.02 | C |
| ATOM | 69 | 9 | C | LEU F | 43 | −16.335 | 31.576 | 6.997 | 1.00 | 18.42 | C |
| ATOM | 70 | 0 | O | LEU F | 43 | −16.752 | 32.686 | 7.346 | 1.00 | 18.68 | O |
| ATOM | 70 | 1 | N | ASP F | 44 | −15.329 | 31.412 | 6.137 | 1.00 | 19.34 | N |
| ATOM | 70 | 2 | CA | ASP F | 44 | −14.683 | 32.538 | 5.459 | 1.00 | 20.62 | C |
| ATOM | 70 | 3 | CB | ASP F | 44 | −13.516 | 32.053 | 4.593 | 1.00 | 20.67 | C |
| ATOM | 70 | 4 | CG | ASP F | 44 | −12.775 | 33.194 | 3.912 | 1.00 | 21.67 | C |
| ATOM | 70 | 5 | OD1 | ASP F | 44 | −12.197 | 34.042 | 4.630 | 1.00 | 22.97 | O |
| ATOM | 70 | 6 | OD2 | ASP F | 44 | −12.765 | 33.244 | 2.660 | 1.00 | 22.25 | O |
| ATOM | 70 | 7 | C | ASP F | 44 | −15.663 | 33.329 | 4.595 | 1.00 | 21.38 | C |
| ATOM | 70 | 8 | O | ASP F | 44 | −15.659 | 34.561 | 4.612 | 1.00 | 21.33 | O |
| ATOM | 70 | 9 | N | ARG F | 45 | −16.494 | 32.617 | 3.838 | 1.00 | 22.47 | N |
| ATOM | 71 | 0 | CA | ARG F | 45 | −17.481 | 33.262 | 2.972 | 1.00 | 23.70 | C |
| ATOM | 71 | 1 | CB | ARG F | 45 | −18.069 | 32.264 | 1.967 | 1.00 | 23.79 | C |
| ATOM | 71 | 2 | CG | ARG F | 45 | −17.048 | 31.674 | 0.984 | 1.00 | 23.98 | C |
| ATOM | 71 | 3 | CD | ARG F | 45 | −16.216 | 32.749 | 0.278 | 1.00 | 24.74 | C |
| ATOM | 71 | 4 | NE | ARG F | 45 | −17.015 | 33.553 | −0.643 | 1.00 | 25.25 | N |
| ATOM | 71 | 5 | CZ | ARG F | 45 | −16.601 | 34.587 | −1.341 | 1.00 | 30.69 | C |
| ATOM | 71 | 6 | NH1 | ARG F | 45 | −15.413 | 35.113 | −1.067 | 1.00 | 31.30 | N |
| ATOM | 71 | 7 | NH2 | ARG F | 45 | −17.379 | 35.172 | −2.244 | 1.00 | 31.41 | N |
| ATOM | 71 | 8 | C | ARG F | 45 | −18.582 | 33.956 | 3.777 | 1.00 | 24.67 | C |
| ATOM | 71 | 9 | O | ARG F | 45 | −18.954 | 35.090 | 3.472 | 1.00 | 24.99 | O |
| ATOM | 72 | 0 | N | LEU F | 46 | −19.086 | 33.278 | 4.808 | 1.00 | 25.62 | N |
| ATOM | 72 | 1 | CA | LEU F | 46 | −20.098 | 33.856 | 5.692 | 1.00 | 26.58 | C |
| ATOM | 72 | 2 | CB | LEU F | 46 | −20.878 | 32.759 | 6.423 | 1.00 | 26.78 | C |
| ATOM | 72 | 3 | CG | LEU F | 46 | −21.911 | 31.956 | 5.627 | 1.00 | 27.60 | C |
| ATOM | 72 | 4 | CD1 | LEU F | 46 | −22.556 | 30.898 | 6.511 | 1.00 | 28.21 | C |
| ATOM | 72 | 5 | CD2 | LEU F | 46 | −22.978 | 32.863 | 5.020 | 1.00 | 28.32 | C |
| ATOM | 72 | 6 | C | LEU F | 46 | −19.486 | 34.826 | 6.699 | 1.00 | 27.08 | C |
| ATOM | 72 | 7 | O | LEU F | 46 | −20.134 | 35.793 | 7.115 | 1.00 | 27.59 | O |
| ATOM | 72 | 8 | N | NH2 F | 47 | −18.286 | 35.191 | 6.835 | 1.00 | 27.50 | N |

### EXAMPLE 3

### Additional Information Regarding the High Resolution Structure of M2 Transmembrane Region

A high-resolution crystallographic structure (1.65 Å) was obtained of a peptide (M2TM′ hereafter) spanning the TM helical region (residues 25-46) of the M2 protein from the Udorn strain with the mutation G34A. The structure reveals a proton conduction path composed of alternating layers of sidechains and well-ordered water clusters. QM/MM studies shed light on the mechanism by which these waters achieve the observed stabilization of the excess charge, and classical MD calculations provide insight into the selectivity and rectification of the translocation process.

As described more fully below, crystallographic electron density maps of the high resolution M2TM′ G34A structure and the medium resolution M2TM′ G34 model are highly similar; therefore, for optimal drug design, the crystallographic structure of the M2 protein with the G34A mutation may be altered in silico to remove the methyl side chain on the Alanine residue that resulted from the mutation, thereby converting the Alanine residue to a Glycine residue and restoring the model to the wild-type state. This method has been successfully employed by the inventors for drug design.

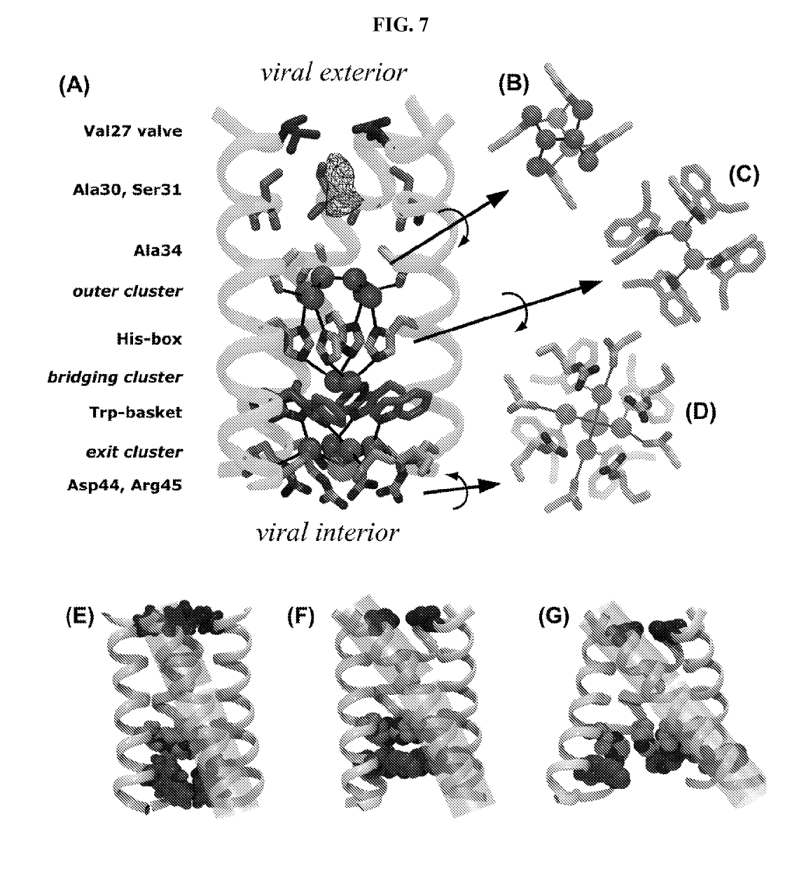

FIG. 7 provides a representation of the high-resolution crystallographic structure of the portion of the M2 transmembrane peptide spanning residues 25-46 (M2TM′). The backbone of three monomers is shown in FIG. 7A, and the pore-lining side chain groups are highlighted as “sticks”. From the N-terminus (viral exterior) to the C-terminus (viral interior), these are: the Val27 valve , Ala30 and Ser31, the mutated residue Ala34, the His-box, the Trp-basket, and the Asp/Arg-box. Water molecules belonging to the outer, bridging, and exit clusters are represented by spheres. Black lines indicate the observed water-protein H-bonds. In the exit cluster the fifth water molecule (showing a high Debye-Waller factor) is drawn transparent and with a larger radius. The wireframe represents the peak region of the diffuse electron density detected right under the Val27 valve. FIGS. 7B-D provide a second perspective of the outer (B), bridging (C), and exit (D) clusters viewed normal to the membrane plane. FIGS. 7E-G show the M2TM helix bundle in different experimental structures, with Val27, His37 and Trp41 in evidence: (E) transmembrane portion of the previously reported NMR structure at pH 7.5 (J. R. Schnell, J. J. Chou, Nature 451, 591 (2008)), (F) the X-ray structure at pH 6.5 presented here, and (G) the previously reported X-ray structure at pH 5.3 (A. L. Stouffer et al., Nature 452, 380 (2008)). The cylinders highlight, respectively, the N- and C-terminal portions of the helices and their angle with respect to the bundle axis. In the X-ray structure presented here the C-terminal portion shows the same angle as the NMR structure (J. R. Schnell & J. J. Chou), while the N-terminal one closely resembles that of the previous X-ray structure (A. L. Stouffer et al.).

M2TM′ was crystallized at pH ˜6.5, giving crystals that diffract to 1.65 Å. As in previous work (A. L. Stouffer et al.), Gly34 was converted to Ala to assist forming high-quality crystals. An equivalent peptide without this mutation gave crystals in the same space group, but diffraction was weaker. Diffraction data sets for M2TM crystals were collected at synchrotron beam line X29 (NSLS, Brookhaven, N.Y., USA). The data sets were processed to ˜68% completeness and belonged to the same unit cell as M2TM′ data sets, with unit cell parameters within the range observed for different individual crystals of M2TM′. To assess the similarity between the structure of M2TM′ and M2TM, the dataset was submitted to molecular replacement using the structure of M2TM′ with the Ala 34 methyl removed. A satisfactory solution (R_{cryst}=0.41, CC=0.65 (polyala)) was obtained, resulting in a map of good quality. Clear density from sidechains not included in the polyalanine molecular replacement model was observed. For example, His 37 sidechains were observed in the same position as in the highly refined M2TM′ G34A density map.

A second molecular replacement solution utilized the crystal structure of M2TM′ G34A with the Ala 34 methyl removed and all solvent molecules also removed. Rigid body refinement gave a solution with Rcryst/Rfree 0.299/0.333. Density that could be assigned to water molecules was observed in the pore in the vicinity of His 37 at the same location as the G34A M2TM′ structure.

These findings indicate that the overall structures are similar, and the high resolution structure of G34A is a useful guiding model when the Ala is mutated to Gly in silico.

M2TM′ assembles into a nearly symmetrical helical bundle, structurally similar to previous lower-resolution models (A. L. Stouffer et al.; J. R. Schnell & J. J. Chou; J. Hu et al., Biophys. J. 92, 4335 (2007); C. Tian, P. F. Gao, L. H. Pinto, R. A. Lamb, T. A. Cross, Protein Sci. 12, 2597 (2003); L. H. Pinto et al., Proc. Natl. Acad. Sci. U.S.A. 94, 11301 (1997)). However, the significantly greater resolution now shows that the pore is formed by five layers of sidechains and three intercalated water clusters stacked to form a continuous conduction pathway (FIG. 7A). The outermost or “top” layer of side chains is composed by the four Val27 residues, which form a nearly closed Val27 valve (2 Å pore radius), leading to a central pore lined by small residues, Ala30, Ser31, and Gly/Ala34. The conduction pathway is next interrupted by the His37 residues (forming what we term a His-box, similar to aromatic boxes, but smaller in cross-section due to the smaller size of the imidazole ring). The His-box needs to expand only slightly (1-2 Å) to allow passage of a water-sized molecule. Below this motif the sidechains of Trp41 form a basket (Trp-basket) with the aromatic rings angled by approximately 45° relative to the bundle axis. Finally, Asp44 and Arg45 line an Asp/Arg box, defining the cytoplasmic end of the channel. The planar faces of the guanidino groups of Arg45 form a 7 to 8 Å box stabilized at the corners by interaction with an oxygen atom of Asp44. While the electron density for the Asp44 residues is very well-defined, the Arg45 sidechains have higher Debye-Waller factors, suggestive of greater conformational mobility.

Directly below the Val27 valve is a region of diffuse density, indicative of dynamically or statically disordered solvent (FIG. 7A); beyond this point, the remainder of the pore is filled by three well-ordered water layers. Above the His-box is an outer cluster of 6 waters (FIG. 7A, B) that consists of a tight water dimer (O—O distance 2.4 Å) atop four waters, which in turn are H-bonded to the N_{δ} of His37 and the backbone carbonyl of residue 34. In the following, we refer to the outer cluster and the His-box together as the “multi-proton storage” cluster (MPSC). Connecting the His-box and Trp-basket is the His37/Trp41 bridging cluster of 2 waters (FIG. 7A, C), which H-bonds to the N_{ε} of each His37 residue. This dimer is well positioned to mediate a π-cation interaction (A. Okada, T. Miura, H. Takeuchi, Biochemistry 40, 6053 (2001)) between charged His37 residues and the electron-rich faces of Trp41 residues. Finally, the exit cluster (FIG. 7A, D) consists of four waters that form H-bonds connecting the indole NH of Trp41 to a carboxylate O of Asp44. A fifth, poorly ordered solvent molecule (presumably water) lies below these four waters, displaced towards the interior of the virus. Throughout the structure, the four-fold symmetry is broken only by the water dimer found in the outer cluster and the His37/Trp41 bridging cluster (FIG. 7B, C). No counterion was detected in the structure, although the density in the central pore around Ala30 and Ser31, as well as the fifth solvent in the exit cluster is sufficiently diffuse that it would be difficult to unambiguously rule out disordered chloride ions with partial occupancy at these positions.

In summary, the pore of M2TM′ is populated by water molecules stably H-bonded to the protein, starting below the Val27 valve, extending through the MPSC, and broken only at the π-face of Trp41, near the interior of the virus.

Peptide synthesis and sample preparation. The A/M2 25-46 G34A peptide (M2TM′, 4-bromobenzoyl-PLVVAASIIAILHLILWILDRL-CONH₂) and the corresponding wildtype peptide with Gly at position 34 (designated here M2TM) were synthesized using Fmoc chemistry on an Applied Biosystems 433A and a Protein Technology Symphony synthesizer as previously described (Stouffer et al, 2008). 4-bromobenzoic acid (Sigma Aldrich, St. Louis, Mo.) was coupled to the amino terminus of the peptide on resin in N,N-dimethylformamide, with HATU activation. The product was cleaved from the resin and purified as previously described (Stouffer et al, 2008). Purity was verified with analytical RP-HPLC and MALDI-TOF mass spectrometry. To make aliquots for crystallization, peptide and n-octylglucoside (OG, Sigma Aldrich) were mixed in an aqueous:isopropanol (1:1) stock using ε280_{peptide}=5853 M⁻¹ cm⁻¹. The mixture was evaporated under reduced pressure. The resulting film was taken up in 5% (w/v) xylitol and mixed with precipitant for crystallization trials. Synthesis and sample preparation of the G34 wild-type M2TM′ peptide were performed in a similar fashion.

Crystallization of M2TM′ (G34A mutant) and M2TM (wild-type sequence). The protein was crystallized using the hanging drop method. A drop containing 0.8 mM M2TM′ peptide (as monomer), 28 mM OG and 5% xylitol was mixed in the ratio of 1:1 with a reservoir solution mixture of 95% [100 mM sodium citrate pH 5.6, 150 mM trisodium citrate, 15% v/v isopropanol] and 5% [0.2M MgCl₂ 6H₂O, 0.1M Trishydrochloride pH 8.5, 30% w/v Polyethlene glycol 4000]. The reservoir solution for the M2TM peptide (reconstituted similarly to M2TM′) was 100 mM sodium citrate pH 5.6, 100 mM tri-sodium citrate, 15% v/v isopropanol. M2TM′ crystals began appearing in two weeks, and were grown over a period of 2-5 months. M2TM crystals appeared after 5 months and were grown for an additional two months.

Data collection and processing. Many data sets were collected from several crystals of M2TM′, with cryo-cooling to 100K during data collection. These include MAD data sets recorded at synchrotron beam lines (NSLS, Brookhaven, N.Y., USA) and data sets collected at a home source. The crystals were usually found to diffract to a maximum resolution of 2.6-1.65Å. The diffraction images were indexed, integrated using MOSFLM and scaled using SCALA. Table 4, below, shows the data collection statistics for the data sets (1.65 Å) used in the refinement.

TABLE 4
Data collection, processing and refinement statistics for M2TM′

| Data collection and | | | | |
|---|---|---|---|---|
| processing | M2TM′G34A | | M2TM′G34 | |
| Space group | C222₁ | | C222₁ | |
| Cell dimensions | 48.67, 79.09, 48.56 | | 48.74, 77.860, 48.61 | |
| a, b, c (Å) | | | | |
| Resolution (Å) | 48.6-1.65 | (1.75-1.65)¹ | 41.3-2.50 | (2.64-2.5) |
| Rmerge | 0.070 | (0.45) | 0.098 | (0.344) |
| I/σ(I) | 19.4 | (2.7) | 7.1 | (2.3) |
| Completeness (%) | 99.5 | (96.6) | 67.5 | (70.9) |
| Multiplicity | 6.6 | (4.5) | 2.8 | (2.7) |

| | | |
|---|---|---|
| Total number of | 76869/11567 | 6174/2230 |
| (observation/unique) | | |
| Refinement | | |
| Resolution (Å) | 31.5-1.65 | 41.3-2.5 |
| Number of reflections | 10998 | 2130 |
| R_{work}/R_{free} | 0.196/0.205 | 0.299/0.333 |
| Number of atoms | | |
| Proteins | 732² | 728 |
| Ligand/ion | 31 | 0 |
| Waters | 25 | 0 |
| B-factors (Å²) | | |
| Proteins | 13.7 | 13.2 |
| Ligand/ion | 48.4 | |
| Waters | 24.9 | |
| R.M.S. deviation | | |
| Bond lengths (Å) | 0.007 | |
| Bond angle (°) | 0.879 | |
| Ramachandran plot | | |
| Residues in | | |
| Most favorable region | 100.0 | 100.0 |
| (%) | | |
| Additional allowed | 0.0 | 0.0 |
| region (%) | | |
| Generously allowed | 0.0 | 0.0 |
| region (%) | | |

¹Highest resolution shell is shown in parenthesis.
²Including Bromo benzoyl group.
³R_{work} = Σ ||F_{obs}| − |F_{calc}||/Σ|F_{obs}| where F_{obs} and F_{calc} are calculated observed and calculated structure factor amplitudes respectively, R_{free} was calculated as R_{work} using 5.0% of the randomly selected unique reflections that were not included in the structure refinement.

Diffraction data sets for M2TM crystals were collected at synchrotron beam line X29 (NSLSL, Brookhaven, N.Y., USA). The data sets could be processed up to ˜68% completeness and was found belong to the same unit cell as M2TM′ data sets (Table 4), and it also showed unit cell parameters within the range observed for different individual crystals of M2TM′. However, crystals were not stable enough to obtain a complete data. set. To assess the similarity between the structure of M2TM′ and M2TM, the partial dataset was submitted to molecular replacement using the structure of M2TM′. A satisfactory solution (R_{cryst}=0.41, CC=0.65 (polyala)) was obtained, resulting in a map of good quality. These findings strongly suggest that the overall structures are similar, although it would be inappropriate to refine the structure of M2TM without collecting more complete data.

Structure solution, model building and refinement of M2TM′. Initial attempts to determine the structure by experimental phasing (MAD, SAD) were not successful, due to the high disorder associated with bromine atom positions. Molecular replacement using a single alpha-helix or tetramers from a previously solved structure (PDB code 3BKD) as model probes did not yield the structure solution. By comparison of cell dimensions, it was intuitive to notice that helices forming the tetramer are more closely packed in the present data sets than those in 3BKD. Whereas in 3BKD (with cell dimensions a=38.753 Å, b=56.557 Å and c=56.009Å), helices are oriented along the a crystallographic axis, and two tetramers lie in the bc plane, in the present data sets (Table 4), tetramer(s) lie(s) in the ac plane with helices oriented along the b axis. Based on this packing knowledge, tetramer models were generated to use as model probes in molecular replacement.

Tetramer model generation. Using one of the helices of our previously published 2.0 Å resolution structure (3BKD, chain ‘A’), tetramer models were generated with various helical orientations; −30°<Xangle<−20°, 7° <Yangle<10°, 160°<Zangle<190° and radius of bundle 6.8<r<8.0 Å, where Xangle, Yangle and Zangle represents the angle formed by the helix with the X-axis, Y-axis and Z-axis respectively. During tetramer generation, the helix oriented along the Z-axis was first rotated around the Z-axis (Zangle), then about the X-axis (Xangle) and then about the Y-axis (Yangle). For this orientated helix, four copies were created by applying four fold symmetry along the Zaxis and each copy was translated to a radius distance in four fold symmetry on a XY plane.

Among tetramer models generated as described above, only those models having orientation of Val27 close to that in the 3BKD structure were considered for molecular replacement calculations. The molecular replacement calculations were performed using the program PHASER for data in the resolution range 15-4.0 Å. The data sets used for initial phasing were processed in the space group P2₁ with cell parameters a=46.41 Å, b=48.52 Å, c=46.27 Å and β=116.97° and were collected at the home source to a resolution limit of 2.6 Å. The Matthews coefficient (Vₘ=2.3 Å³/Da) indicated the presence of two tetramers in the asymmetric unit. Initially molecular replacement calculations were performed using bundles with parameters: radius 8.0 Å, 7.5 Å [−26° (Xangle), 10° (Yangle), 160°-190° (Zangle)] and later, models were randomly selected for the calculations. In evaluating model quality during calculations, importance was given to recognizing an interpretable electron density map. In general, the resulting maps were not good, but the model with Xangle=−24°, Yangle=10°, Zangle=167°, and radius 7.0 Å had a very clear density for a dimer. The dimer was used as model and two tetramers were located in the asymmetric unit, resulting in an excellent electron density map.

Of several crystals tested for diffraction at the synchrotron beam line (NSLS, X6A), a crystal belonging to the space group C222₁ with the cell parameters a=48.67 Å b=79.09 Å and c=48.56 Å was found to diffract to a maximum resolution of 1.65 Å. With the possibility of a tetramer in the asymmetric unit as suggested by the Matthews coefficient (Vₘ=2.4 Å³/Da), the structure was solved by the molecular replacement technique using one of the above-determined tetramers as a model. Iterative refinement and model building were carried out, during which all side chains could be traced in the electron density map. As the refinement progressed water molecules were located in the map. When the refinement was converged, the model was refined to R_{work}=19.6% and R_{free}=20.5% (Table 4), with all the residues in allowed regions (100% favorable) of the Ramachandran plot. Details of refinement statistics are shown in Table 4. The refinement was carried out using the CCP4 program suite (CCP4, 1994) REFMAC and all model building was done using COOT.

In general, solvent molecules were included only if they were visible at the 3-sigma level in an F₀-F_{C} map. All were found in full occupancy with the exception of the Br atoms in the bromobenzoyl group, which were disordered, presumably due to radiation damage. The dimer of water molecules in the outer cluster had two alternate conformations that refined with occupancies of 0.6 and 0.4.

Classical MD simulations. Classical molecular dynamics (MD) simulations were performed for 7 protonation states of M2TM′ (neutral, 1ε, 2ε, 2 δ, 3ε, 3 δ, 4). In addition, simulations were performed on the 2ε state of the wild type and of the mutants G34V and S31N. All the calculations were done using the NAMD software package and the CHARMM forcefield. The system was contained in a periodic box, with the electrostatic potential being solved by the particle mesh Ewald (PME) method with an accuracy threshold of 10₋₆ and a real space spherical cutoff of 12 Å. Lennard-Jones interactions were cut off at 12 Å. The equations of motion were solved with the velocity Verlet integrator using a timestep of 1.5 fs. The length of all the bonds involving hydrogen atoms were constrained with the SHAKE method. The system was run at 310.0 K and 1.013 bars using Langevin temperature and Langevin piston pressure coupling schemes. Only the dimension of the box perpendicular to the membrane was allowed to vary. Decay times for the thermostat and barostat were chosen to be 1 ps and 0.1 ps respectively. The M2TM′ protein was solvated in 5707 TIP3P water molecules. The heavy atoms of the peptide backbone were restrained to remain near the positions determined in the crystal structure with a harmonic force (k=20.0 kcal/mol/Å₂) for the G34A simulations. These restraints were lifted in an additional simulation of the 2ε state of M2TM′ and in the simulations of all the other mutants of M2TM. The system was initially equilibrated for 1.125 ns, and then data was collected over a molecular dynamics run of 15 ns. Calculations of the free energy profile of the pore water dipoles were also performed by metadynamics. One collective variable was used, defined as the average among the pore waters of the z-projection of the water molecule dipole in the point charge representation, <d>=<q(H1)*z(H1)+q(H2)*z(H2)+q(O)*z(O)>. In Debye (D) units, its value can range between −2.347 D and 2.347 D, because we used the rigid TIP3P water model. The number of pore waters ranged from 14 in the neutral state to 22 in the 4+ state. Gaussian functions of 0.01 kcal/mol weight and 0.05 D width were used, inserted with a frequency of 0.2 ps. Each metadynamics simulation was run until states at >10 kcal/mol free energy were explored (on average, about 50 ns for each protonation state). In the final stages of each simulation, a variance of <1 kcal/mol in the free energy at a given value of <d> was observed, which indicates good statistical convergence.

Analysis of the pore waters' structure. On the 15 ns MD trajectory of each protonation state, we computed the water density profiles by counting the number of waters in the pore within intervals 0.25 Å wide of the z Cartesian coordinate. The average number of H-bonds between these waters was also computed as a function of z, and plotted for three regions of the pore. Trajectory frames every 3 ps were included in the analysis.

To analyze the distribution of H-bonded water wires, the distribution of mutual RMSDs between any two H-bonds vectors in the trajectory was calculated. In each protonation state, this distribution has a peak at around 0.6 Å, which represents the fluctuation of H-bond vectors between “structured” waters. This peak vanishes at around 1.2 Å, where the distribution first has a saddle and then increases rapidly when the larger RMSD values between H-bonds involving different pairs of waters are included. The 1.2 Å value was used as the cutoff for a cluster analysis of the H-bonds, performed using the method implemented for Gromos. The distributions of H-bond clusters for the most significant protonation states was plotted.

The crystallographic structures disclosed herein are in excellent agreement with a wide body of functional and spectroscopic data and provide a basis for the design of new inhibitors that target amantadine-resistant mutants of M2 (in addition to the wild-type M2 protein) Inhibitors that target the cavity defined by the residues 27, 30, 31, 34, 37, 41, 44, and 45 might reclaim the M2-blocking class of drugs both for prophylaxis and for treatment of ongoing endemic outbreaks and future pandemics of this deadly pathogen.

Additional information pertaining to the present invention may be found in Stouffer A L, Acharya R, Salom D, Levine A S, Di Costanzo L, Soto C S, Tereshko V, Nanda V, Stayrook S, DeGrado W F. Nature. 2008 Jan 31; 451(7178):596-9 and the supplemental materials pertaining thereto; and, Acharya R, Carnevale V, Fiorin G, Levine B G, Polishchuk A L, Balannik V, Lamb R A, Pinto L H, DeGrado W F, and Klein M L, Influenza A Virus Employs Water Clusters to Sequester Charge in a Biological Membrane. Submitted to Science, Jun. 9, 2009, and the supplemental materials pertaining thereto, all of which are hereby incorporated by reference in their entirety.

What is claimed:

1. A method for identifying a compound that modulates the activity of influenza A comprising:
comparing spatial models of a plurality of test compounds with a spatial model of the proton transport pathway of the tetrameric M2 transmembrane protein of the influenza A virus;
said pathway being defined by at least two residues from among residues 27, 30, 31, 34, 37 or 41, 44, and 45 on one or more subunits of the protein;
determining the spatial complementarity of each of the test compounds with said pathway;
assessing the ability of said test compounds to bind to said pathway; and,
based on the assessed ability of said test compounds to bind to said pathway, determining said compound that modulates the activity of influenza A.

2. The method according to claim 1 wherein said proton transport pathway is defined, at least in part, by the same residue on two or more of said subunits.

3. The method according to claim 1 wherein said proton transport pathway is defined by at least two residues on a single subunit of said protein.

4. The method according to claim 1 wherein said proton transport pathway is defined by at least one residue on one of said subunits and at least one residue on another of said subunits.

5. The method according to claim 1 wherein said spatial models of said test compounds and said spatial model of said proton transport pathway are computer-based.

6. The method according to claim 1 wherein said spatial model of said proton transport pathway comprises at least a portion of the tetrameric four-helix bundle of said M2 protein.

7. The method according to claim 1 wherein said spatial model of said proton transport pathway comprises the transmembrane region of a wild-type M2 protein.

8. The method according to claim 1 wherein said spatial model of said proton transport pathway is defined by at least three residues from among residues 27, 30, 31, 34, 37, 41, 44, and 45 on one or more subunits of the protein.

9. The method according to claim 1 wherein said spatial model of said proton transport pathway comprises the transmembrane region of an M2 protein having a mutation at one or more of said residues 27, 30, 31, 34, 37, 41, 44, and 45.

10. The method according to claim 1 wherein said spatial model of said proton transport pathway comprises the transmembrane region of an M2 protein having a mutation that does not prevent the ability of a corresponding M2 protein to transport a proton across a membrane.

11. The method according to claim 1 wherein said spatial model of said proton transport pathway comprises the transmembrane region of an M2 protein having the V27G mutation, the V27I mutation, the V27T mutation, the V27S mutation, or the V27A mutation.

12. The method according to claim 1 wherein said spatial model of said proton transport pathway comprises the transmembrane region of an M2 protein having the A30T mutation.

13. The method according to claim 1 wherein said spatial model of said proton transport pathway comprises the transmembrane region of an M2 protein having the S31A mutation or the S31N mutation.

14. The method according to claim 1 wherein said spatial model of said proton transport pathway comprises the transmembrane region of an M2 protein having the G34E mutation or the G34A mutation.

15. The method according to claim 1 wherein said spatial model of said proton transport pathway comprises the transmembrane region of an M2 protein having the W41L mutation or the W41Y mutation.

16. The method according to claim 1 wherein said spatial model of said proton transport pathway comprises the transmembrane region of an M2 protein having the D44N mutation or the D44H mutation.

17. The method according to claim 1 wherein said spatial model of said proton transport pathway comprises the transmembrane region of an M2 protein having the R45K mutation or the R45H mutation.

18. The method according to claim 1 wherein said spatial model of said proton transport pathway is further defined by a water molecule within about 5 Angstroms of any of said residues.

19. The method according to claim 1 further comprising testing said determined compound in an influenza A inhibition assay.

20. The method according to claim 19 wherein said testing comprises an in vitro influenza A inhibition assay.

21. The method according to claim 19 wherein said testing comprises assessing the ability of said compound to modulate the activity of the M2 transmembrane protein.

22. A method for evaluating the ability of a test compound to modulate the activity of influenza A comprising:
comparing a spatial model of said test compound with a spatial model of the proton transport pathway of the tetrameric M2 transmembrane protein of the influenza A virus;
said pathway being defined by at least two residues from among residues 27, 30, 31, 34, 37 or 41, 44, and 45 on one or more subunits of the protein;
determining the spatial complementarity of the test compound with said pathway;
assessing the ability of said test compound to bind to said pathway; and,
based on the assessed ability of said test compound to bind said pathway, determining whether said compound modulates the activity of influenza A.

23. The method according to claim 22 wherein said proton transport pathway is defined, at least in part, by the same residue on two or more of said subunits.

24. The method according to claim 22 wherein said proton transport pathway is defined by at least two residues on a single subunit of said protein.

25. The method according to claim 22 wherein said proton transport pathway is defined by at least one residue on one of said subunits and at least one residue on another of said subunits.

26. The method according to claim 22 wherein said spatial models of said test compounds and said spatial model of said proton transport pathway are computer-based.

27. The method according to claim 22 wherein said spatial model of said proton transport pathway comprises at least a portion of the tetrameric four-helix bundle of said M2 protein.

28. The method according to claim 22 wherein said spatial model of said proton transport pathway comprises the transmembrane region of a wild-type M2 protein.

29. The method according to claim 22 wherein said spatial model of said proton transport pathway is defined by at least three residues from among residues 27, 30, 31, 34, 37, 41, 44, and 45 on one or more subunits of the protein.

30. The method according to claim 22 wherein said spatial model of said proton transport pathway comprises the transmembrane region of an M2 protein having a mutation at one or more of said residues 27, 30, 31, 34, 37, 41, 44, and 45.

31. The method according to claim 22 wherein said spatial model of said proton transport pathway comprises the transmembrane region of an M2 protein having a mutation that does not prevent the ability of a corresponding M2 protein to transport a proton across a membrane.

32. The method according to claim 22 wherein said spatial model of said proton transport pathway comprises the transmembrane region of an M2 protein having the V27G mutation, the V27I mutation, the V27T mutation, the V27S mutation, or the V27A mutation.

33. The method according to claim 22 wherein said spatial model of said proton transport pathway comprises the transmembrane region of an M2 protein having the A30T mutation.

34. The method according to claim 22 wherein said spatial model of said proton transport pathway comprises the transmembrane region of an M2 protein having the S31A mutation or the S31N mutation.

35. The method according to claim 22 wherein said spatial model of said proton transport pathway comprises the transmembrane region of an M2 protein having the G34E mutation or the G34A mutation.

36. The method according to claim 22 wherein said spatial model of said proton transport pathway comprises the transmembrane region of an M2 protein having the W41L mutation or the W41Y mutation.

37. The method according to claim 22 wherein said spatial model of said proton transport pathway comprises the transmembrane region of an M2 protein having the D44N mutation or the D44H mutation.

38. The method according to claim 22 wherein said spatial model of said proton transport pathway comprises the transmembrane region of an M2 protein having the R45K mutation or the R45H mutation.

39. The method according to claim 22 wherein said spatial model of said proton transport pathway is further defined by a water molecule within about 5 Angstroms of any of said residues.

40. The method according to claim 22 further comprising testing said determined compound in an influenza A inhibition assay.

41. The method according to claim 40 wherein said testing comprises an in vitro influenza A inhibition assay.

42. The method according to claim 40 wherein said testing comprises assessing the ability of said compound to modulate the activity of the M2 transmembrane protein.
